(19) United States
(12) Patent Application Publication
(10) Pub. No.: US 2024/0384318 A1
(43) Pub. Date: Nov. 21, 2024

(54) COMPOSITIONS AND METHODS FOR ENZYMATIC NUCLEIC ACID SYNTHESIS

(71) Applicant: Primrose Bio, Inc., San Diego, CA (US)

(72) Inventors: Daniel OLSON, San Diego, CA (US); Dongxin Karen XU, San Diego, CA (US); Sabrina BAFFERT, San Diego, CA (US); Helge ZIELER, Encinitas, CA (US)

(21) Appl. No.: 18/569,883

(22) PCT Filed: Jun. 13, 2022
(86) PCT No.: PCT/US2022/033313

§ 371 Date: Jun. 14, 2024

Related U.S. Application Data
(60) Provisional application No. 63/210,429, filed on Jun. 14, 2021.

(51) Int. Cl. C12P 19/34; C12N 9/12

(52) U.S. Cl. CPC C12P 19/34; C12N 9/1252; C12Y 207/07007

(57) ABSTRACT

The present disclosure describes compositions and methods useful for the template independent enzymatic synthesis of nucleic acids.

### GOVERNMENT LICENSE RIGHTS

This invention was made with government support under Award Number1R43HG010995-01A1 and Unique Federal Award Identification Number (FAIN) R43HG010995 awarded by the National Institutes of Health. The government has certain rights in the invention.

### INCORPORATION OF SEQUENCE LISTING

The content of the electronically submitted sequence listing in ASCII text file (PG0020_sequence_listing_revised_10-28-21_ST25.txt), which is about 173 KB in size was created on Oct. 28, 2021 and electronically submitted via ePCT on Jun. 13, 2022.

### BACKGROUND

Chemical oligonucleotide synthesis (COS), the current method of producing synthetic DNA and RNA, is almost 40 years old and has become limiting for new discoveries in fields such as functional genomics, synthetic biology, DNA-based data storage, and medical applications that rely on rapid and inexpensive DNA synthesis. The cost of COS has only improved by 20× over the last quarter century (see, for example, the data displayed for the bioeconomy dashboard on the Bioeconomy Capital web site) and has not kept up with the rising demand for synthetic DNA. Furthermore, COS is limited to nucleic acid strands having up to or around 200 nucleotides, and requires large, centralized facilities that employ sophisticated equipment and production processes. The rapidly rising demand for synthetic nucleic acids calls for new, rapid and inexpensive synthesis routes capable of delivering long nucleic acid molecules. Because of the abundance of DNA and RNA polymerases in nature, enzymatic nucleic acid synthesis routes are receiving much attention.

Enzymatic oligonucleotide synthesis (EOS) has been pursued by various commercial groups for several years (Efcavitch 2016, Hiatt 1995, Hiatt 1995a), with recent exciting discoveries and advances (Palluk 2018, Perkel 2019, Hoff 2020, Lee 2020). Such strategies can be aimed at making either RNA or DNA oligonucleotides, or RNA-DNA chimeras.

Most EOS strategies use terminal deoxynucleotidyl transferases (TdTs) which are template-independent DNA polymerases (TIDPs) capable of adding nucleotides to the 3′ ends of single-stranded DNA in vitro (Deibel 1980, Fowler 2006, Motca 2010, Jensen 2018, Loc'h 2018, Deshpande 2019, Sarac 2019). Known TdTs will polymerize DNA hundreds of nucleotides long (Deibel 1980, Delarue 2002, Fowler 2006, Motca 2010, Jensen 2018, Loc'h 2018, Sarac 2019), cither through high processivity or through a high on-off rate of the enzyme (Gouge 2013). Other DNA polymerases, especially ones involved in DNA repair processes, have also been shown to have template-independent DNA polymerase (TIDP) activity in vitro (Clark 1988, Domínguez 2000, Ruiz 2001, Juárez 2006, Moon 2007, Moon 2007a, Hogg 2012, Moon 2014, Kent 2016, Frank 2017, Yang 2018, Chang 2019), although the TIDP activity of non-TdT enzymes has not been studied extensively.

To produce polynucleotides of defined length and sequence, current EOS processes usc 3′-blocked nucleotides, with removal of the blocking group after each addition cycle (FIG. 1A). The 3′ blocking group prevents the addition of multiple nucleotides per addition cycle.

However, 3′-blocked nucleotides have a number of drawbacks that limit progress in this field. First, most natural DNA polymerases incorporate nucleotides with 3′ modifications very inefficiently and also display marked base preference and sequence specificity. Second, the chemical nature of the 3′ blocking group is critical because it needs to be at the same time sufficiently stable to avoid spontaneous or enzyme-catalyzed removal during the addition step and completely removable to prepare for the next addition step. This balance is difficult to strike and has limited the field to a small number of blocking group chemistries that have the desirable qualities. Third, the enzyme needs to accommodate the 3′ blocking group which creates an interconnected challenge of nucleotide chemistry and enzyme optimization. Fourth, the deblocking step of this strategy adds a chemical reaction step to an otherwise enzymatic synthesis process, increasing the process complexity and potentially involving the use of expensive and toxic chemicals.

An alternative approach to oligonucleotide synthesis has been described that uses natural or unblocked nucleoside triphosphates (Schott 1984). Because of the processive addition of multiple nucleotides by template-independent nucleic acid polymerases, this method requires that after each addition cycle, oligonucleotide molecules that received a single nucleotide addition are separated from oligonucleotides that received 0, 2 or more nucleotides. The requirement for oligonucleotide purification after each addition cycle has limited the utility of this method.

To simplify the problem of enzymatic oligonucleotide synthesis and create a differentiated approach for an efficient enzymatic oligonucleotide synthesis process, we developed the strategy shown in FIG. 1B which uses only natural nucleotides. A TIDP that efficiently adds a nucleotide and then fails to translocate and remains associated with the DNA template, will reliably add only a single nucleotide per synthesis cycle. The enzyme thereby prevents the addition of more than one nucleotide to the 3′ end of an oligonucleotide substrate and obviates the need for modified nucleotides. Before initiating a new cycle, the nucleotides are removed and the enzyme is dissociated by washing, heating and/or with chaotropic salts. The evolution of TIDPs suited for this process is greatly streamlined and DNA synthesis cost will be much reduced. Primordial Genetics' cost models show that such an EOS process will have a 10×-100× cost advantage over COS at small (fmol) and medium (nmol-μmol) synthesis scales.

The present disclosure demonstrates feasibility for this unique DNA synthesis approach using a set of first-generation DNA synthesis enzymes with the ability to incorporate a single nucleotide into the end of a single-stranded oligonucleotide.

The commercial opportunities in this space are vast as applications for synthetic DNA are growing rapidly. The global oligonucleotide synthesis market size was $4.3B in 2018 and is expected to grow at 10-12.5% Compound Annual Growth Rate (CAGR) to reach >$8.0 billion by 2025 (Global Oligonucleotide Synthesis Market Size 2018). The main applications for synthetic DNA include molecular and synthetic biology R&D, genomics (target enrichment), therapeutics, diagnostics (DNA microarrays, PCR and FISH), CRISPR/Cas9 systems, nanotechnology and emerging technologies such as DNA-based data storage and DNA computing (Global Oligonucleotide Synthesis Market Size 2018, Lec 2018, Jensen 2018, Lec 2019).

The present disclosure describes a novel enzymatic route to oligonucleotide synthesis using nucleoside triphosphates with free or unblocked 3′ hydroxyl groups as substrates, referred to hereafter as ‘unblocked nucleoside triphosphates.’ DNA polymerases with TIDP activity that have been described to date typically show processive addition of nucleotides to single-stranded oligonucleotide or polynucleotide ends when reacted in vitro together with triphosphates. The present disclosure describes DNA polymerases with the ability to add a single nucleotide to the 3′ end of an oligonucleotide when used together with unblocked nucleoside triphosphates.

The disclosure is firmly rooted in known DNA polymerase mechanisms. In brief, all DNA polymerases are known undergo six key mechanistic steps (Berdis 2009, Beard 2014, Berdis 2014): 1) Polymerase binding to the DNA substrate; 2) Formation of an initial ternary complex with the nucleoside triphosphate; 3) Conformational changes leading to a productive ternary substrate complex; 4) Catalysis leading to a post-chemistry product ternary complex; 5) Conformational changes leading to product (PPi) release, and 6) Polymerase translocation to prepare for the next round of nucleotide addition or polymerase dissociation from the DNA substrate. Various of these mechanistic steps are mediated by different domains of the polymerase (Kaminsky 2020).

Polymerase translocation is known to be associated with specific DNA polymerase sequences and domains (Samkurashvili 1996, Rechkoblit 2006, Golosov 2010, Dahl 2014, Ren 2016, Yang 2018, Hoitsma 2020), and polymerases with widely different rates of dissociation from their substrates have been reported (Andrade 2009, Zahn 2011). Mutations have been identified in both DNA and RNA polymerases that affect the translocation rate (Samkurashvili 1996, Dahl 2014, Ren 2016), and polymerase translocation has been associated with specific domains and sequence motifs found in DNA and RNA polymerases (Samkurashvili 1996, Rechkoblit 2006, Golosov 2010, Dahl 2014, Hoitsma 2020). It is therefore possible to develop a nucleic acid polymerase that adds a single unblocked nucleotide and fails to add others due to an inability to translocate.

Nucleic acid polymerases fall into different classes, with polymerases within a class exhibiting specific sequences or properties that distinguish them from polymerases within another class. For example, DNA polymerases are classified into families A, B, C, D, X, Y and RT (Bebenck 2002, Ramadan 2004, Jarosz 2007, Guo 2009, Uchiyama 2009, Yamtich 2010, Berdis 2014, Maxwell 2014, Moon 2014, Trakselis 2014, Yang 2014, Vaisman 2017, Yang 2018, Hoitsma 2020, Kazlauskas 2020). Polymerases in different families have different biological functions in nucleic acid replication, repair and recombination. Purified polymerases from different families often have distinct sets of activities in vitro as exemplified in the references listed above.

Nucleic acid polymerases are also known to exhibit strong sequence specificity or preference for specific sequences in polymerizing nucleic acids. Nucleic acid polymerases have also been shown to exhibit base specificity when polymerizing nucleic acids (Fiala 2007, Hoitsma 2020).

Based on the known qualities of DNA polymerases, there are various potential ways to achieve addition of a single nucleotide to the 3′ end of a single-stranded nucleic acid molecule without risking processive addition of multiple nucleotides, including but not limited to: 1) Use of a polymerase with high sequence specificity for the 3′ end sequence of the nucleic acid molecule that is modified; this end sequence specificity may or may not be coupled to a base specificity in terms of the polymerase's preference to incorporate a specific type of nucleotide (i.e. A, C, G, T, U or I); 2) Use of a DNA polymerase that is unable to translocate after nucleotide addition (step 6 above) and that remains associated with the 3′ end of the nucleic acid molecule after nucleotide addition; 3) Combinations thereof; and 4) Other mechanisms that allow TIDPs to act non-processively on a nucleic acid substrate and only add a single unblocked nucleotide in a template-independent manner.

### BRIEF SUMMARY

The present disclosure describes a novel approach to enzymatic de novo synthesis of nucleic acids which involves addition of single nucleotides to a nucleic acid substrate by template-independent nucleic acid polymerases (TINAPs) without the use of 3′ blocking groups on the nucleoside triphosphate monomers. This disclosure also describes enzymes capable of adding single nucleotides to the 3′ end of a nucleic acid in a template-independent manner. This surprising finding contradicts the progressive manner in which DNA polymerases are known and thought to operate. As a result, such enzymes, or modified derivatives thereof, find utility in the development of EOS processes that require controlled addition of nucleotides to the 3′ end of a nucleic acid, one nucleotide at a time. The disclosure describes the use of such enzymes in processes used for synthesizing nucleic acids for industrial, medical, diagnostic, agricultural, and/or R&D use.

### BRIEF DESCRIPTION OF THE FIGURES

FIG. 1A. Schematic representation of enzymatic oligonucleotide synthesis by cyclical addition of 3′-blocked nucleotides to an oligonucleotide (see Jensen 2018). An oligonucleotide coupled to a bead (top left) is combined with a 3′-blocked nucleoside triphosphate (top) and an enzyme (top right) which catalyzes the addition of a nucleotide to the bead. After removal of the enzyme and excess nucleoside triphosphates (not shown), the 3′ protecting group is cleaved off (bottom), leaving a free 3′ end that is the substrate of another addition. When the synthesis is complete, the deprotected oligonucleotide can be cleaved off the bead (bottom left). The diagram shows addition of a C residue to a DNA oligo but applies equally to any nucleotide added to any RNA or DNA oligonucleotide, or modified forms or chimeras thereof.

FIG. 1B. Schematic representation of enzymatic oligonucleotide synthesis by cyclical addition of nucleotides to an oligonucleotide, showing how elimination of the protecting group can simplify the nucleic acid synthesis cycle.

FIG. 1C. Schematic representation of enzymatic oligonucleotide synthesis by cyclical addition of unblocked nucleotides to an oligonucleotide. An oligonucleotide coupled to a bead (top left) is combined with a nucleoside triphosphate with a free 3′ end (top) and an enzyme (top right) which catalyzes the addition of a single nucleotide to the bead. After removal of the enzyme (bottom left) and excess nucleoside triphosphates (not shown), the cycle can be repeated. When the synthesis is complete, the oligonucleotide can be cleaved off the bead (bottom left). The diagram shows addition of a C residue to a DNA oligo but applies equally to any nucleotide added to any RNA or DNA oligonucleotide, or modified forms or chimeras thereof.

FIG. 1D. Schematic representation of enzymatic oligonucleotide synthesis by cyclical addition of unblocked nucleotides to an oligonucleotide, showing one possible mechanism by which a single nucleotide is added per addition cycle. An oligonucleotide coupled to a bead (top left) is combined with a nucleoside triphosphate with a free 3′ end (top) and an enzyme (top right) which catalyzes the addition of a single nucleotide to the bead. After nucleotide addition, the enzyme remains bound to the 3′ end of the oligonucleotide, preventing further nucleic acid polymerization. After removal of the enzyme (bottom left) and excess nucleoside triphosphates (not shown), the cycle can be repeated. When the synthesis is complete, the oligonucleotide can be cleaved off the bead (bottom left). The diagram shows addition of a C residue to a DNA oligo but applies equally to any nucleotide added to any RNA or DNA oligonucleotide, or modified forms or chimeras thereof.

FIG. 2: Results of nucleotide addition reactions involving a mix of oligonucleotide substrates (SEQ ID NOs: 42-45) with mixed nucleoside triphosphates (equimolar mixture of dATP, dCTP, dGTP and dTTP). A single stranded DNA ladder is shown in the “M” lanes, containing molecule sizes as indicated by the labels on the left of the gel image. The EDS numbers of the enzymes tested, which are identifiers used for all enzymes listed in this disclosure (see Table 1 for details), are shown below the gel image. The enzymes tested show addition of varying lengths of sequences to the substrates.

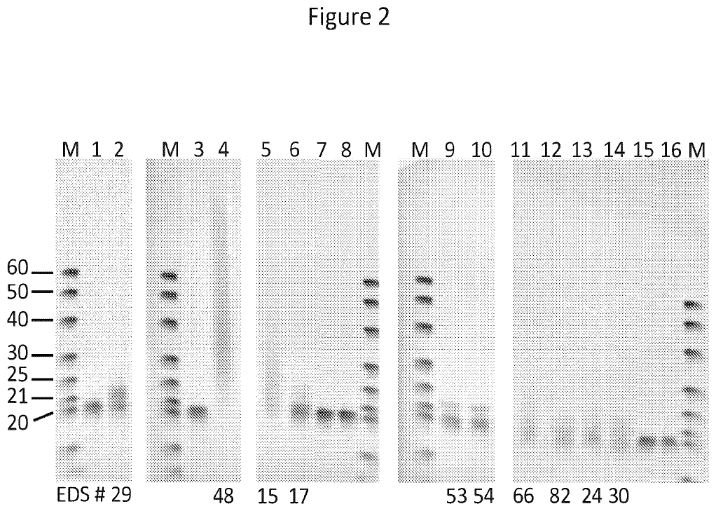

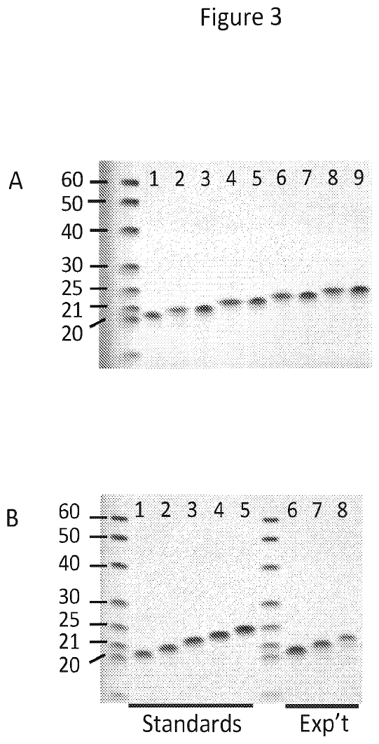

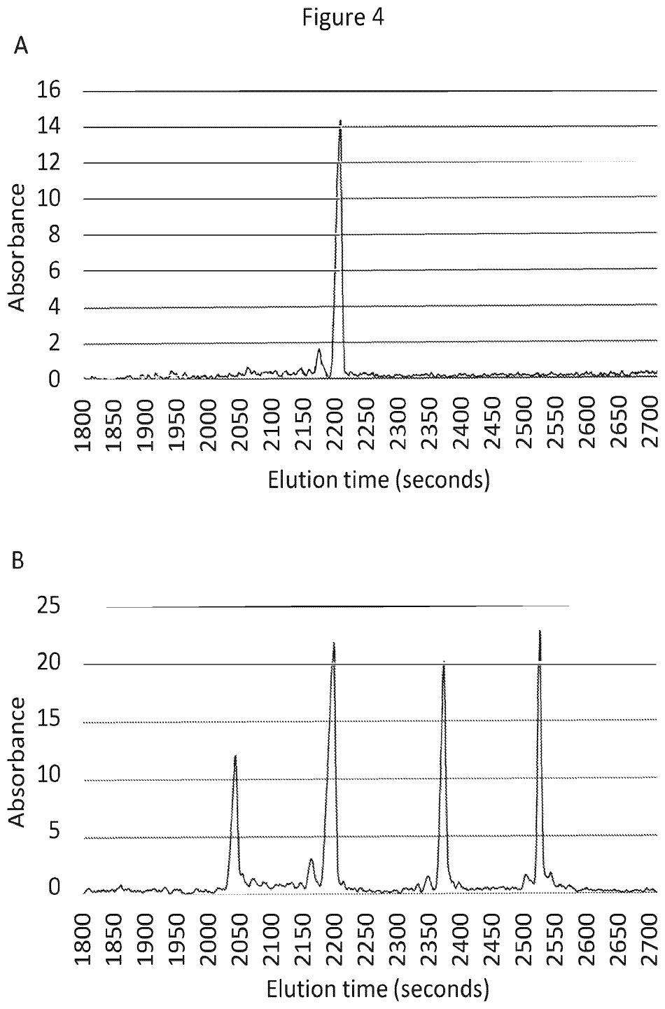

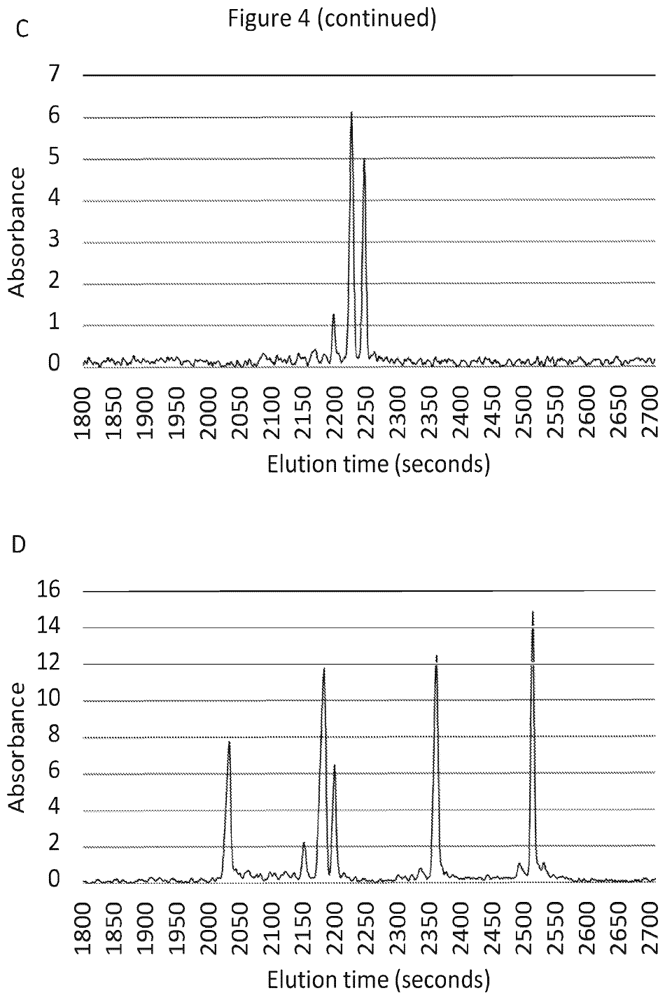

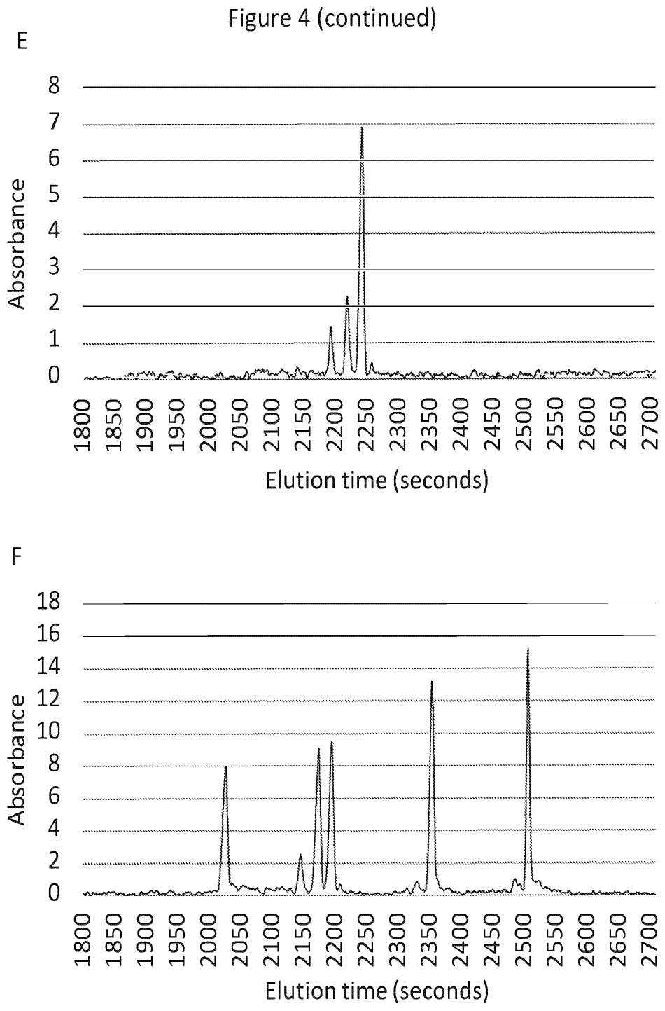

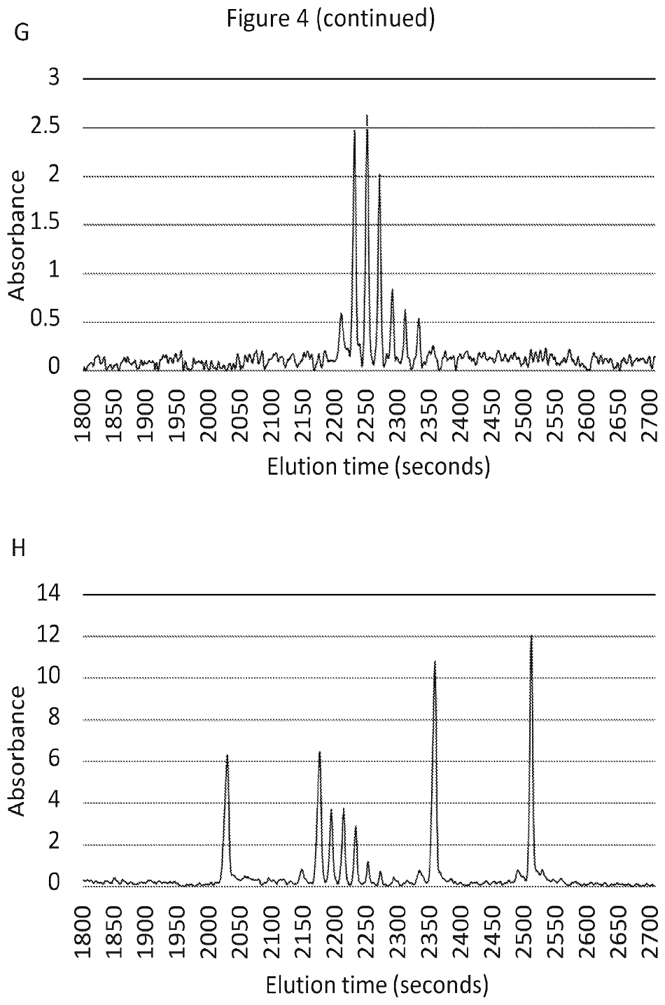

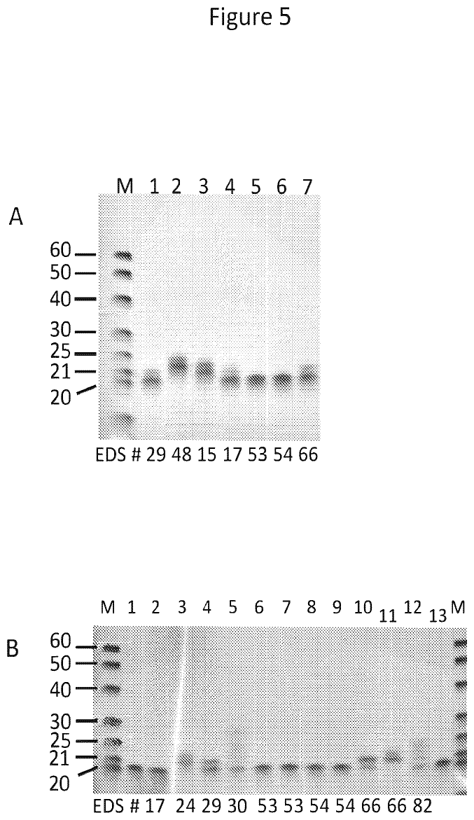

FIG. 2 legend: Assay for DNA polymerase ability to add 1
nucleotide to a single-stranded oligonucleotide substrate

| | | | | Starting | |
|---|---|---|---|---|---|
| Lane | Oligonucleotide | Enzyme | 3′ end | length | Final length |
| # | substrate | used | base | (nucleotides) | (nucleotides) |
| M | single stranded | None | N.A. | N.A. | N.A. |
| | DNA ladder | | | | |
| 1 | SEQ ID NOs: | None | A, C, | 20 | 20 |
| | 42-45 | | G, T | | |
| 2 | SEQ ID NOs: | EDS029 | A, C, | 20 | 22 |
| | 42-45 | | G, T | | |
| M | single stranded | None | N.A. | N.A. | N.A. |
| | DNA ladder | | | | |
| 3 | SEQ ID NOs: | None | A, C, | 20 | 20 |
| | 42-45 | | G, T | | |
| 4 | SEQ ID NOs: | EDS048 | A, C, | 20 | 60+ |
| | 42-45 | | G, T | | |
| 5 | SEQ ID NOs: | EDS015 | A, C, | 20 | 40+ |
| | 42-45 | | G, T | | |
| 6 | SEQ ID NOs: | EDS017 | A, C, | 20 | 25 |
| | 42-45 | | G, T | | |
| 7 | SEQ ID NOs: | None | A, C, | 20 | 20 |
| | 42-45 | | G, T | | |
| 8 | SEQ ID NOs: | None | A, C, | 20 | 20 |
| | 42-45 | | G, T | | |
| M | single stranded | None | N.A. | N.A. | N.A. |
| | DNA ladder | | | | |
| M | single stranded | None | N.A. | N.A. | N.A. |
| | DNA ladder | | | | |
| 9 | SEQ ID NOs: | EDS053 | A, C, | 20 | 21 |
| | 42-45 | | G, T | | |
| 10 | SEQ ID NOs: | EDS054 | A, C, | 20 | 21 |
| | 42-45 | | G, T | | |
| 11 | SEQ ID NOs: | EDS066 | A, C, | 20 | 25 |
| | 42-45 | | G, T | | |
| 12 | SEQ ID NOs: | EDS082 | A, C, | 20 | 23 |
| | 42-45 | | G, T | | |
| 13 | SEQ ID NOs: | EDS024 | A, C, | 20 | 25 |
| | 42-45 | | G, T | | |
| 14 | SEQ ID NOs: | EDS030 | A, C, | 20 | 23 |
| | 42-45 | | G, T | | |
| 15 | SEQ ID NOs: | None | A, C, | 20 | 20 |
| | 42-45 | | G, T | | |
| 16 | SEQ ID NOs: | None | A, C, | 20 | 20 |
| | 42-45 | | G, T | | |
| M | single stranded | None | N.A. | N.A. | N.A. |
| | DNA ladder | | | | |

FIG. 3: Results of controlled addition of single nucleotides to oligonucleotide substrates terminating in different bases. A. Addition of single nucleotides to different oligonucleotide substrates, assayed by gel following the reaction. A single stranded DNA ladder is shown in the leftmost lane, containing molecule sizes as indicated by the labels on the left of the gel image. B. Sequential addition of two nucleotides to an oligonucleotide substrate with purification of the oligonucleotide after the first addition step. A single stranded DNA ladder is shown to the left of lane 1 and to the left of lane 6, containing molecule sizes as indicated by the labels on the left of the gel image. The column in the table below labeled “3′ end base” lists the 3′ terminal base of the major oligonucleotide present in each lane.

FIG. 3A legend: Enzymatic addition of single nucleotides
to different oligonucleotide substrates

| | | | 3′ | Expected | Observed |
|---|---|---|---|---|---|
| Lane | | Enzyme | end | length | length |
| # | Oligonucleotide | used | base | (nucleotides) | (nucleotides) |
| 1 | SEQ ID NO: 45 | None | T | 20 | 20 |
| 2 | SEQ ID NO: 45 + | EDS054 | T | 21 | 21 |
| | T added | | | | |
| 3 | SEQ ID NO: 46 | None | T | 21 | 21 |
| 4 | SEQ ID NO: 46 + | EDS053 | G | 22 | 22 |
| | G added | | | | |
| 5 | SEQ ID NO: 47 | None | G | 22 | 22 |
| 6 | SEQ ID NO: 47 + | EDS054 | A | 23 | 23 |
| | A added | | | | |
| 7 | SEQ ID NO: 48 | None | A | 23 | 23 |
| 8 | SEQ ID NO: 48 + | EDS082 | C | 24 | 24 |
| | C added | | | | |
| 9 | SEQ ID NO: 54 | None | C | 24 | 24 |

FIG. 3B legend: Sequential enzymatic addition of nucleotides to an oligonucleotide template

| | | Enzyme | | Expected length | Observed length |
|---|---|---|---|---|---|
| Lane # | Oligonucleotide | used | 3′ end base | (nucleotides) | (nucleotides) |
| 1 | SEQ ID NO: 45 | None | T | 20 | 20 |
| 2 | SEQ ID NO: 46 | None | T | 21 | 21 |
| 3 | SEQ ID NO: 47 | None | G | 22 | 22 |
| 4 | SEQ ID NO: 48 | None | A | 23 | 23 |
| 5 | SEQ ID NO: 54 | None | C | 24 | 24 |
| 6 | SEQ ID NO: 45 | None | T | 20 | 20 |
| 7 | SEQ ID NO: 45 + T added | EDS054 | T | 21 | 21 |
| 8 | SEQ ID NO: 45 + T + G added | EDS053 | G | 22 | 22 |

FIG. 4: Representative capillary electrophoresis separation chromatograms of oligonucleotides before and after enzymatic nucleotide addition, performed on an Oligo Pro II capillary electrophoresis instrument (Agilent Technologies, Santa Clara, CA). All reactions shown in the chromatograms used dTTP and Oligo: PG5861 (GTCCTCAATCGCACTGGAAT, SEQ ID NO: 45). For unambiguous assignment of lengths to the oligonucleotides present in each sample, duplicate analysis of the sample with and without Oligonucleotide Standards was conducted. Oligonucleotide Standards used were PG1350 (GCGTCACGCTACCAACCA, SEQ ID NO: 41); PG5861 (GTCCTCAATCGCACTGGAAT, SEQ ID NO: 45); PG5870 (GTCCTCAATCGCACTGGAAACATCAAGGTC, SEQ ID NO: 51); and PG5871 (GTCCTCAATCGCACTGGAAACATCAAGGTCATACGGAACG, SEQ ID NO: 52). A: Unreacted (i.e. no enzyme) oligonucleotide PG5861 (GTCCTCAATCGCACTGGAAT, SEQ ID NO: 45). B: Unreacted (i.e. no enzyme) oligonucleotide PG5861

(GTCCTCAATCGCACTGGAAT, SEQ ID NO: 45) combined with Oligonucleotide Standards. C: Oligonucleotide PG5861 (GTCCTCAATCGCACTGGAAT, SEQ ID NO: 45) reacted with dTTP and enzyme EDS082. D: Oligonucleotide PG5861 (GTCCTCAATCGCACTGGAAT, SEQ ID NO: 45) reacted with dTTP and enzyme EDS082, combined after the reaction with Oligonucleotide Standards. E: Oligonucleotide PG5861 (GTCCTCAATCGCACTGGAAT, SEQ ID NO: 45) reacted with dTTP and enzyme EDS054. F: Oligonucleotide PG5861 (GTCCTCAATCGCACTGGAAT, SEQ ID NO: 45) reacted with dTTP and enzyme EDS054, combined after the reaction with Oligonucleotide Standards. G: Oligonucleotide PG5861 (GTCCTCAATCGCACTGGAAT, SEQ ID NO: 45) reacted with dTTP and enzyme EDS066. H: Oligonucleotide PG5861 (GTCCTCAATCGCACTGGAAT, SEQ ID NO: 45) reacted with dTTP and enzyme EDS066, combined after the reaction with Oligonucleotide Standards.

FIG. 5: Results of nucleotide addition reactions showing the addition of varying lengths of sequences to the substrates. A: oligonucleotide substrates (SEQ ID NOs: 42-45) with an equimolar mixture of ATP, CTP, GTP and UTP and enzymes EDS015, EDS017, EDS029, EDS048, EDS053, EDS054, or EDS066. A single stranded DNA ladder is shown in the “M” lane, containing molecule sizes as indicated by the labels on the left of the gel image. B: a single oligonucleotide substrate (SEQ ID NO 45) with an equimolar mixture of ATP, CTP, GTP and UTP and enzymes EDS017, EDS024, EDS029, EDS030, EDS053, EDS054, EDS066, or EDS082. A single stranded DNA ladder is shown in the “M” lanes, containing molecule sizes as indicated by the labels on the left of the gel image.

FIG. 5A legend: Assay for DNA polymerase ability to add ribonucleotides
to a single-stranded deoxyribose oligonucleotide substrate

| | | Enzyme | | Starting length | Final length |
|---|---|---|---|---|---|
| Lane # | Oligonucleotide substrate | used | Nucleotides in reaction | (nucleotides) | (nucleotides) |
| M | single stranded DNA ladder | None | N.A. | NA | NA |
| 1 | SEQ ID NOs: 42-45 | EDS029 | ATP, CTP, GTP, UTP | 20 | 21 |
| 2 | SEQ ID NOs: 42-45 | EDS048 | ATP, CTP, GTP, UTP | 20 | 23 |
| 3 | SEQ ID NOs: 42-45 | EDS015 | ATP, CTP, GTP, UTP | 20 | 22 |
| 4 | SEQ ID NOs: 42-45 | EDS017 | ATP, CTP, GTP, UTP | 20 | 21 |
| 5 | SEQ ID NOs: 42-45 | EDS053 | ATP, CTP, GTP, UTP | 20 | 20 |
| 6 | SEQ ID NOs: 42-45 | EDS054 | ATP, CTP, GTP, UTP | 20 | 20 |
| 7 | SEQ ID NOs: 42-45 | EDS066 | ATP, CTP, GTP, UTP | 20 | 21 |

FIG. 5B legend: Assay for DNA polymerase ability to add ribonucleotides
to a single-stranded deoxyribose oligonucleotide substrate

| | | Enzyme | | Starting length | Final length |
|---|---|---|---|---|---|
| Lane # | Oligonucleotide substrate | used | Nucleotides in reaction | (nucleotides) | (nucleotides) |
| M | single stranded DNA ladder | None | N.A. | NA | NA |
| 1 | SEQ ID NO: 45 | None | ATP, CTP, GTP, UTP | 20 | 20 |
| 2 | SEQ ID NO: 45 | EDS017 | ATP, CTP, GTP, UTP | 20 | 20 |
| 3 | SEQ ID NO: 45 | EDS024 | ATP, CTP, GTP, UTP | 20 | 22 |
| 4 | SEQ ID NO: 45 | EDS029 | ATP, CTP, GTP, UTP | 20 | 21 |
| 5 | SEQ ID NO: 45 | EDS030 | ATP, CTP, GTP, UTP | 20 | 30 |
| 6 | SEQ ID NO: 45 | EDS053 | ATP, CTP, GTP, UTP | 20 | 20 |
| 7 | SEQ ID NO: 45 | EDS053 | ATP, CTP, GTP, UTP | 20 | 20 |
| 8 | SEQ ID NO: 45 | EDS054 | ATP, CTP, GTP, UTP | 20 | 20 |
| 9 | SEQ ID NO: 45 | EDS054 | ATP, CTP, GTP, UTP | 20 | 20 |
| 10 | SEQ ID NO: 45 | EDS066 | ATP, CTP, GTP, UTP | 20 | 22 |
| 11 | SEQ ID NO: 45 | EDS066 | ATP, CTP, GTP, UTP | 20 | 22 |
| 12 | SEQ ID NO: 45 | EDS082 | ATP, CTP, GTP, UTP | 20 | 24 |
| 13 | SEQ ID NO: 45 | None | ATP, CTP, GTP, UTP | 20 | 20 |
| M | single stranded DNA ladder | None | N.A. | NA | NA |

### DETAILED DESCRIPTION

The following abbreviations and definitions will be used for the interpretation of the specification and the claims.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.” For example, a condition A or B is satisfied by any one of the following: A is true (or present) and B is false (or not present), A is false (or not present) and B is true (or present), and both A and B are true (or present).

Addition cycle: As used herein, this phrase refers to one round of nucleotide addition in a nucleic acid synthesis process involving two or more such rounds of addition. In each addition cycle, the single-stranded nucleic acid being synthesized is combined with a nucleoside triphosphate and a nucleic acid polymerase and incubated under reaction conditions in which the nucleic acid polymerase is active, resulting in nucleotide addition to the single-stranded nucleic acid.

Base specificity of nucleic acid polymerases: This phrase refers to the preference of a nucleic acid polymerase to add a nucleotide containing a specific base compared to a different basc. For example, a DNA polymerase with a preference for dTTP will add dTMP (deoxythymidine monophosphate) residues more efficiently to the 3′ end of a nucleic acid than nucleotides containing other bases such as A, C or G. In another example, in a mixed reaction containing equimolar amounts of the nucleoside triphosphates dATP, dCTP, dGTP and dTTP, a DNA polymerase with a preference for dTTP will add a higher number of dTMP residues to the 3′ end of a nucleic acid than nucleotides containing the other three bases A, C or G.

Chimeric nucleic acid: As used herein, chimeric nucleic acid refers to a nucleic acid molecule that contains a mixture of ribonucleotide and deoxyribonucleotide residues. A mixture means that any number of ribonucleotide residues are present in the same nucleic acid strand together with any number of deoxynucleotide residues.

Complementary nucleotide sequence: As used herein, a complementary nucleotide sequence is a polynucleotide sequence in which all of the bases are able to form base pairs with another polynucleotide sequence of the opposite 5′ to 3′ polarity, such that all bases in each polynucleotide chain are paired with their counterpart, forming base pairs.

Control elements: The term ‘control elements’ refers to nucleotide sequences located upstream (5′ non-coding sequences), within, or downstream (3′ non-coding sequences) of a coding sequence and which influence the transcription, RNA processing or stability, or translation of the associated coding sequence. Regulatory sequences include, but are not limited to, promoters, translation leader sequences, introns, polyadenylation recognition sequences, RNA processing sites, effector binding sites, and stem-loop structures.

Degenerate Sequence: In this application degenerate sequences are defined as populations of sequences where specific sequence positions differ between different molecules or clones in the population. The sequence differences may be a single nucleotide or multiple nucleotides of any number, examples being 2, 3, 4, 5, 6, 7, 8, 9, 10, 20, 30, 40, 50, 60, 70, 80, 90, 100, 200, 300, 400, 500, 600, 700, 800, 900, 1000 nucleotides, or any number in between. Sequence differences in a degenerate sequence may involve the presence of 2, 3 or 4 different nucleotides in that position within the population of sequences, molecules or clones. Examples of degenerate nucleotides in a specific position of a sequence are: A or C; A or G; A or T; C or G; C or T; G or T; A, C or G; A, C or T; A, G or T; C, G or T; A, C, G or T.

DNA: DNA is a nucleic acid that is a polymer of deoxyribonucleotides. DNA occurs in single stranded or double stranded forms. As used herein, DNA contains nucleotide residues each of which has a 2′ carbon in the form CH2.

Enzymatic oligonucleotide synthesis (EOS): As used herein, is a controlled enzymatic process of synthesizing nucleic acids using stepwise enzymatic addition of single nucleotides to the end of a nucleic acid, thus creating a new nucleic acid one nucleotide at a time.

Expression: The term “expression”, as used herein, refers to the transcription and stable accumulation of sense (mRNA) or antisense RNA derived from the nucleic acid disclosed, as well as the accumulation of polypeptide as a product of translation of mRNA.

Free nucleotide: As used herein, means a monomeric nucleotide, typically in solution.

Full-length Open Reading Frame: As used herein, a full-length open reading frame refers to an open reading frame encoding a full-length protein which extends from its natural initiation codon to its natural final amino-acid coding codon, as expressed in a cell or organism. In cases where a particular open reading frame sequence gives rise to multiple distinct full-length proteins expressed within a cell or an organism, each open reading frame within this sequence, encoding one of the multiple distinct proteins, are considered full-length. A full-length open reading frame can either be continuous or interrupted by introns.

Full-length Protein: As used herein, a full-length protein is a polypeptide which extends from its natural first amino acid to its natural final amino acid, as encoded in the genome of a cell or organism and expressed in the cell or organism.

Gene: The term “gene” refers to a nucleic acid fragment that is capable of being expressed as a specific protein, optionally including regulatory sequences preceding (5′ non-coding sequences) and following (3′ non-coding sequences) the coding sequence. “Native gene” refers to a gene as found in nature in its natural host organism. “Natural gene” refers to a gene complete with its natural control sequences such as a promoter and terminator. “Chimeric gene” refers to any gene that comprises regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. Similarly, a “foreign” gene refers to a gene not normally found in the host organism, but that is introduced into the host organism by gene transfer. Foreign genes include native genes inserted into a non-native organism, or chimeric genes. A “transgene” is a gene that has been introduced into the genome by a transformation procedure.

In-Frame: The term “in-frame” in this application, and particularly in the phrase “in-frame fusion polynucleotide,” refers to the reading frame of codons in an upstream or 5′ polynucleotide or ORF as being the same reading frame as the reading frame of codons in a polynucleotide or ORF placed downstream or 3′ of the upstream polynucleotide or ORF that is fused with the upstream or 5′ polynucleotide or ORF. Such in-frame fusion polynucleotides encode a fusion protein or fusion peptide encoded by both the 5′ polynucleotide and the 3′ polynucleotide.

In vitro transcription reaction: An “in vitro transcription reaction” as used hercin is a reaction designed to produce RNA by transcribing a DNA template in vitro. In vitro transcription reactions contain one or more DNA template molecules encoding the RNAs to be transcribed, one or more completely or partially purified single-subunit RNA polymerases, a minimum of four nucleoside triphosphates as substrates for the single-subunit RNA polymerase(s), buffers, divalent cations and salts as necessary for the reaction.

Iterate/Iterative: In this application, to iterate means to apply a method or procedure repeatedly to a material or sample. Typically, the processed, altered or modified material or sample produced from cach round of processing, alteration or modification is then used as the starting material for the next round of processing, alteration or modification. Iterative selection refers to a selection process that iterates or repeats the selection two or more times, using the survivors of one round of selection as starting material for the subsequent rounds.

Library: A library of genes or polynucleotide sequences is a collection of sequences that are different from each other and that are cloned into a vector for propagation of the sequences. In different libraries, the sequences differ by sequence content, origin, source organism, length, structure, association with other sequences, and/or any other property of a polynucleotide sequence.

For example, a library of amino acid repeat fusion genes is generated by cloning a starting ORF collection that contains multiple different ORFs encoded by the E. coli genome into a bacterial cloning and expression vector that contains a promoter, a sequence encoding an amino acid repeat oriented in a manner that this sequence will be joined directly and in-frame to the ORFs, a terminator, a plasmid backbone and an antibiotic resistance gene. The starting ORF collection can contain any number of ORFs that number 5 or greater, for example 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000 or greater, or any number in between. In a specific aspect of the disclosure, the ORF collection used to generate the library contains a sufficient number of ORFs to give a high likelihood of encoding a specific desirable property of E. coli, for example 50% or more of the ORFs encoded by the E. coli genome, or 2074 or more ORFs when using the annotation of the E. coli strain MG1655 genome annotation prepared by the University of Wisconsin, Madison which lists a total of 4148 ORFs.

Linker sequence: This phrase refers to a polynucleotide sequence or polypeptide sequence separating two polynucleotides or polypeptides in a fusion polynucleotide or fusion polypeptide. For example, a fusion polynucleotide contains two or more ORFs that are separated by a linker sequence, which encodes a peptide which separates the two parts of the polypeptide that results from expression and translation of the fusion polynucleotide. A linker can also separate an epitope tag from a protein or enzyme. Linker sequences can have diverse length and/or sequence composition.

Non-homologous: The term “non-homologous” in this application is defined as having sequence identity at the nucleotide level of less than 50%.

Nucleic acid: The term nucleic acid refers to biopolymers, consisting of nucleotides joined to each other via phosphodiester linkages, phosphorothioate linkages or other linkages. “Nucleic acid” or “Nucleic acid molecule” can be used interchangeably with polynucleotide. As used herein, the term nucleic acid refers to a single strand of nucleic acid. A nucleic acid can either consist of deoxyribonucleotide residues, in which case it is DNA, or ribonucleotide residues, in which case it is RNA, or it can contain both deoxyribonucleotide residues and ribonucleotide residues in which case it is a chimeric nucleic acid.

Nucleic Acid Substrate or Substrate Nucleic Acid Molecule: This is a nucleic acid molecule present in an enzymatic nucleotide addition reaction or an enzymatic nucleic acid synthesis reaction that serves as the nucleotide acceptor during a reaction catalyzed by a nucleic acid polymerase and using a nucleoside triphosphate as a source of nucleotides. For example, a single-stranded DNA oligonucleotide reacted in the presence of an enzyme and one or more deoxynucleoside triphosphates is the substrate nucleic acid molecule in this reaction.

Nucleic Acid Polymerase”: This is an enzyme that catalyzes the polymerization of a nucleic acid using nucleoside triphosphates and unblocked nucleic acids as substrates and sequentially adds single nucleotides to the 3′ end of the unblocked nucleic acid. Nucleic acid polymerases as described in the scientific literature typically fall into the classes of DNA polymerases and RNA polymerases, with DNA polymerases capable of polymerizing DNA and RNA polymerases capable of polymerizing RNA. However, specific enzymes may have the dual ability to catalyze the synthesis of both DNA and RNA. For example, a DNA polymerase may have the ability to add ribonucleotides to the 3′ end of a DNA or RNA molecule, and an RNA polymerase may have the ability to add deoxyribonucleotides to the 3′ end of a DNA or RNA molecule.

Nucleic acid synthesis: This is the process by which nucleic acids are produced in nature or by man, minimally requiring a nucleic acid polymerase, one or more nucleoside triphosphates as monomer building blocks and a nucleic acid substrate.

De novo nucleic acid synthesis: This is used to refer to synthesis of man-made DNA, involving controlled addition of specific nucleotides to a nucleic acid substrate to create a specific sequence and structure of nucleic acid.

Nucleotides: These are the monomer building blocks of nucleic acids, made of three components: a 5-carbon sugar, a phosphate group and a nitrogenous base. The two main classes of nucleotides are deoxyribonucleotides, the building blocks of DNA and ribonucleotides, the building blocks of RNA. If the sugar is ribose, the nucleic acid is RNA; if the sugar is the ribose derivative deoxyribose, the nucleic acid is DNA. As used herein, a deoxyribonucleotide has the group CH₂ as the 2′ carbon in the ribose sugar. All other structures of the 2′ carbon are grouped under the term ribonucleotides. As used herein, a nucleotide can mean a nucleotide residue present within a nucleic acid, a nucleoside monophosphate, a nucleoside diphosphate, a nucleoside triphosphate or any derivative or modification thereof.

Nucleoside triphosphates: “Nucleoside triphosphates” in this application are defined as any of the ribonucleoside triphosphates ATP, CTP, GTP, ITP, UTP and XTP, etc. used in RNA synthesis, or any of the deoxyribonucleoside triphosphates dATP, dCTP, dGTP, dITP, dTTP and dXTP, etc. used in DNA synthesis, or any modified analogs, derivatives or variants thereof, including derivatives containing phosphorothioate linkages. Mixtures of the four canonical nucleoside triphosphates used in DNA synthesis (dATP, dCTP, dGTP, and dTTP) are denoted by the shorthand “dNTP” and Mixtures of the four canonical nucleoside triphosphates used in RNA synthesis (ATP, CTP, GTP, and UTP) are denoted by the shorthand “NTP”.

Oligonucleotide: The term oligonucleotide refers to a single stranded nucleic acid consisting of two or more nucleotides.

Open Reading Frame (ORF): An ORF is defined as any sequence of nucleotides in a nucleic acid that encodes a protein or peptide as a string of codons in a specific reading frame. Within this specific reading frame, an ORF can contain any codon specifying an amino acid, but does not contain a stop codon. The ORFs in a starting collection need not start or end with any particular amino acid. An ORF is either continuous or is interrupted by one or more introns.

Operably linked: The term “operably linked” refers to the association of nucleic acid sequences on a single nucleic acid fragment so that the function of one is affected by the other. For example, a promoter is operably linked with a coding sequence when it is capable of effecting the expression of that coding sequence (i.e., that the coding sequence is under the transcriptional control of the promoter). Coding sequences can be operably linked to regulatory sequences in sense or antisense orientation.

Peptide bond: A “peptide bond” is a covalent bond between a first amino acid and a second amino acid in which the alpha-amino group of the first amino acid is bonded to the alpha-carboxyl group of the second amino acid.

Percentage of sequence identity: The term “percent sequence identity” refers to the degree of identity between any given query sequence, e.g. SEQ ID NO: 10, and a subject sequence. A subject sequence typically has a length that is from about 80 percent to 200 percent of the length of the query sequence, e.g., 80, 81, 82, 83, 84, 85, 86, 87, 88, 89, 90, 91, 93, 95, 97, 99, 100, 105, 110, 115, 120, 130, 140, 150, 160, 170, 180, 190 or 200 percent of the length of the query sequence. A percent identity for any subject nucleic acid or polypeptide relative to a query nucleic acid or polypeptide is determined as follows. A query sequence (e.g. a nucleic acid or amino acid sequence) is aligned to one or more subject nucleic acid or amino acid sequences using the computer program ClustalW (version 1.83, default parameters), which allows alignments of nucleic acid or protein sequences to be carried out across their entire length (global alignment, Chenna 2003).

To determine a percent identity of a subject or nucleic acid or amino acid sequence to a query sequence, the sequences are aligned using Clustal W, the number of identical matches in the alignment is divided by the query length, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher website and at the European Bioinformatics Institute website on the World Wide Web.

Plasmid and Vector: The terms “plasmid” and “vector” refer to genetic elements used for carrying genes which are not a natural part of a cell or an organism. Plasmids typically replicate extrachromosomally as autonomous episomal genetic clements, while vectors can either integrate into the genome or can be maintained extrachromosomally as linear or circular DNA fragments. Plasmids and vectors can be linear or circular, and can consist of single-and/or double-stranded DNA or RNA that is derived from any source. Plasmids and vectors often contain a number of nucleotide sequences from different sources which have been joined or recombined into a unique construction which is useful for introducing polynucleotide sequences into a cell or an organism and expressing genes within an organism. The sequences present on a plasmid or on a vector include but are not limited to: autonomously replicating sequences; centromere sequences; genome integrating sequences; origins of replication; control sequences such as promoters and/or terminators; open reading frames; selectable marker genes such as antibiotic resistance genes; visible marker genes such as genes encoding fluorescent proteins; restriction endonuclease recognition sites; recombination sites; and/or sequences with no apparent or known function.

Polypeptide or protein: The terms “polypeptide” or “protein” denote a polymer composed of a plurality of amino acid monomers joined by peptide bonds. The polymer comprises 10 or more monomers, including 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000 or any number in between.

Promoter: The term “promoter” refers to a DNA sequence capable of controlling the expression of a coding sequence or functional RNA. In general, a coding sequence is located 3′ to a promoter sequence. Promoters can be derived in their entirety from a native gene, and/or can be composed of different elements derived from different promoters found in nature, or even comprise synthetic DNA segments. It is understood by those skilled in the art that different promoters direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

Random/Randomized: as used herein, means made or chosen without method or conscious decision.

RNA: “RNA” is a nucleic acid that is a polymer of ribonucleotides. RNA occurs in single stranded or double stranded forms. As used herein, RNA contains nucleotide residues each of which has a 2′ carbon in a form other than CH₂.

Sequence: As known to those trained in the art, “sequence,” when used in a biological context, can imply the sequence of nucleotides in a nucleic acid or the sequence of amino acids in a protein. As used herein, the term “sequence” has a meaning dependent on the context in which the term is used. For example, when used in the context suggesting nucleic acids such as genome sequences, gene sequences or ORFs, then sequence refers to a nucleotide sequence. In a context suggesting proteins or polypeptides, such as the proteome, proteins or enzymes, sequence refers to amino acid sequence.

Sequence Specific Nucleotide Addition”: as used herein, this is a feature of nucleic acid polymerases that exhibit sequence specificity in their activity. For example, a template-independent DNA polymerase may have sequence specificity that only allows it to add a nucleotide to the 3′ end of a nucleic acid terminating with a dT residue and not to 3′ ends terminating with other nucleotides. Such sequence specificity of nucleic acid polymerases can be partial or complete. If partial, then the DNA polymerase in the example above will add a nucleotide more efficiently to a nucleic acid terminating in a 3′ dT residue, but will also modify nucleic acids terminating in a 3′ dA, dC or dG residue, albeit less efficiently. If complete, then then the DNA polymerase in the example above will add a nucleotide only to a nucleic acid terminating in a 3′ dT residue, and will fail to modify nucleic acids terminating in a 3′ dA, dC or dG residuc.

Template-independent nucleic acid polymerase: A “template-independent nucleic acid polymerase” is an enzyme that catalyzes the incorporation of nucleotides at the 3′-hydroxyl terminus of a nucleic acid, accompanied by the release of inorganic phosphate, in the absence of another nucleic acid strand that is base-paired to the strand being synthesized and that serves as a template for the strand being synthesized. Specifically, template-independent DNA polymerases catalyze polymerization of a DNA strand without use of a template, while template-independent RNA polymerases catalyze polymerization of an RNA strand without use of a template.

Template-independent Nucleic Acid Synthesis: This is a process by which a nucleic acid polymerase catalyzes the polymerization of a nucleic acid without use of a template strand that is base paired to the nucleic acid being synthesized and that serves as the template for the strand being synthesized.

Transformed: The term “transformed” means genetic modification by introduction of a polynucleotide sequence.

Transformation: As used herein the term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

Transformed Organism: A transformed organism is an organism that has been genetically altered by introduction of a polynucleotide sequence into the organism's genome.

Translocation: “Translocation” of a nucleic acid polymerase refers to the movement of the enzyme along the nucleic acid template in the direction of nucleic acid polymerization (5′ to 3′) following the addition of a nucleotide to a nucleic acid substrate. The nucleic acid polymerase translocates along the template or nucleic acid substrate after addition of a nucleotide to the substrate.

Unfavorable Conditions: As used herein, this phrase implies any part of the growth condition, physical or chemical, that results in slower growth than under normal growth conditions, or that reduces the viability of cells compared to normal growth conditions.

Unblocked Nucleic Acid: This phrase means a nucleic acid having a free 3′ hydroxyl group.

Unblocked Nucleotide or Unblocked Nucleoside Triphosphate or Unblocked dNTP or Unblocked NTP: These phrases are used interchangeably and refer to a nucleotide or nucleoside triphosphate with a free 3′ hydroxyl group.

The term “in-frame” in this disclosure, and particularly in the phrase “in-frame fusion polynucleotide” refers to the reading frame of codons in an upstream or 5′ polynucleotide, gene or ORF as being the same as the reading frame of codons in a polynucleotide, gene or ORF placed downstream or 3′ of the upstream polynucleotide, gene, or ORF that is fused with the upstream or 5′ polynucleotide, gene or ORF. Collections of such in-frame fusion polynucleotides can vary in the percentage of fusion polynucleotides that contain upstream and downstream polynucleotides that are in-frame with respect to one another. The percentage in the total collection is at least 10% and can number 10%, 11%, 12%, 13%, 14%, 15%, 20%, 25%, 30%, 35%, 40%, 45%, 50%, 55%, 60%, 65%, 70%, 75%, 80%, 85%, 90%, 95%, 96%, 97%, 98%, 99%, 100% or any number in between.

XTP or dXTP: The term “XTP” or “dXTP” refers to any ribonucleoside triphosphate or any modified form of a naturally occurring ribonucleoside triphosphate used for synthesizing RNA or modified forms of RNA or any deoxyribonucleoside triphosphate or any modified form of a naturally occurring deoxyribonucleoside triphosphate used for synthesizing DNA or modified forms of DNA, respectively.

The present disclosure provides compositions and methods for synthesizing nucleic acids in a template-independent manner. Certain nucleic acid polymerases have the ability to add nucleotides to a free 3′ terminus of a nucleic acid without a template guiding the addition or the type of nucleotide to be added. In this disclosure such polymerases are referred to as having template-independent nucleic acid polymerase (TINAP) activity.

Polymerases with TINAP activity have utility for creating artificial nucleic acids in vitro. For example, a nucleic acid polymerase with TINAP activity can be combined with one or more nucleoside triphosphates and one or more substrate nucleic acids containing a free 3′ hydroxyl group under experimental conditions allowing nucleic acid synthesis (for example, at physiological pH and in the presence of a buffering agent and of divalent cation cofactors, and incubation at temperatures allowing nucleic acid polymerization). The polymerase catalyzes nucleotide addition to the 3′ end in a manner that in a single addition cycle, the 3′ end of the substrate nucleic acid is extended by a single nucleotide. The nucleic acid molecule is then separated from the enzyme and/or from the nucleoside triphosphates, and the cycle repeated. In this manner, any specific nucleic acid sequence can be synthesized in a cyclical manner, one nucleotide at a time.

The ability to synthesize a specific nucleic acid sequence in the strategy described above depends on the ability of the nucleic acid polymerase with TINAP activity to extend the substrate nucleic acid by a single nucleotide per addition cycle. A small subset of nucleic acid polymerases has this ability.

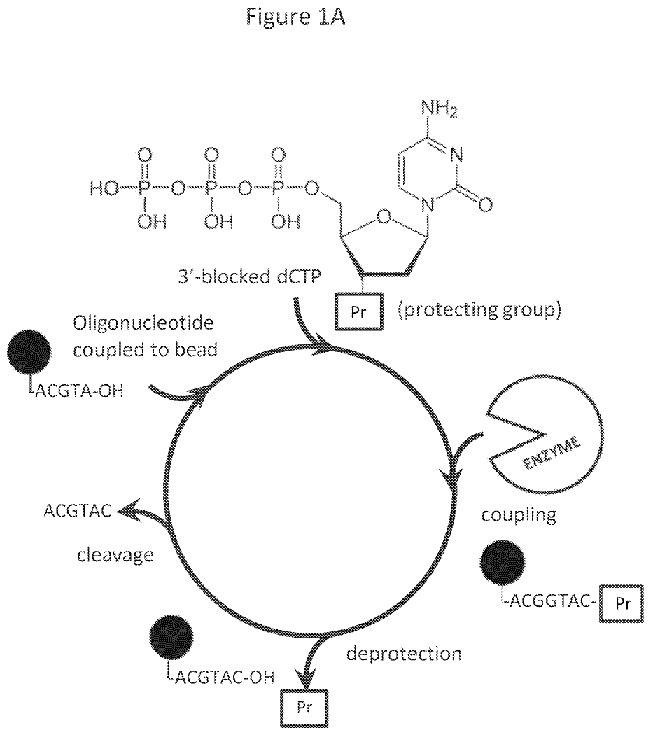

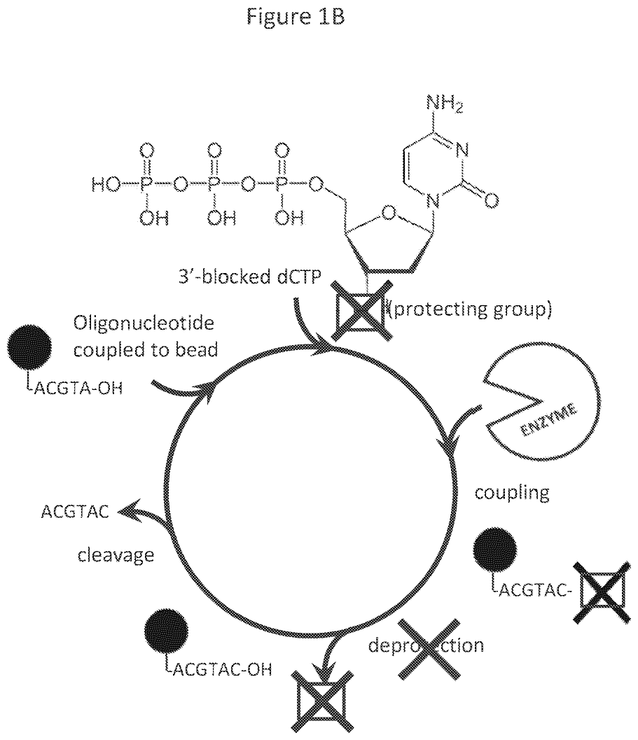

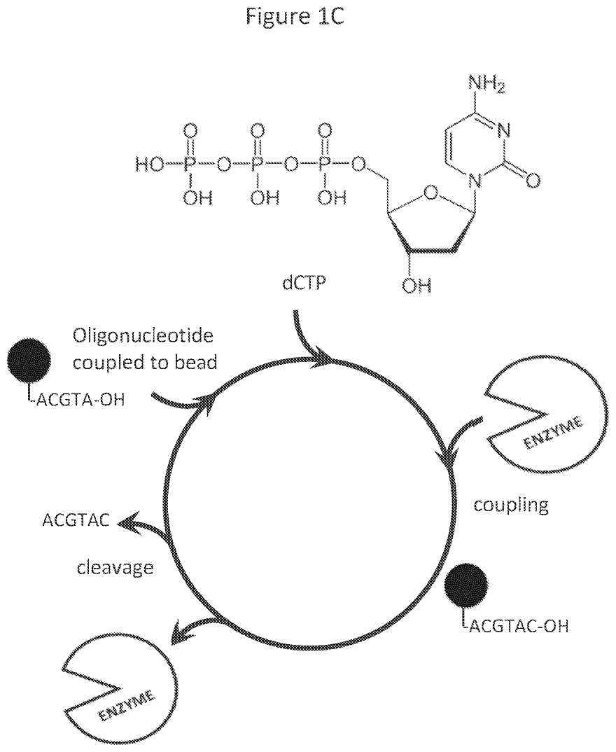

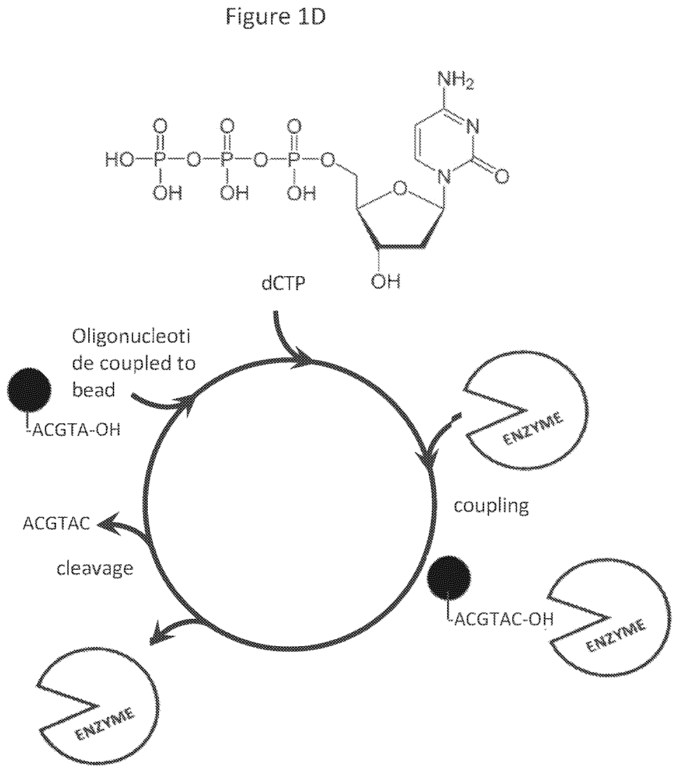

To date, other efforts to develop EOS strategies capable of synthesizing nucleic acids one nucleotide at a time have required the use of 3′-blocked nucleotides, which contain a chemical group covalently linked to the 3′ hydroxyl of the nucleotide being added to the nucleic acid. The chemical blocking group modifying the 3′ hydroxyl prevents the addition of multiple nucleotides to a free 3′ hydroxyl group of a substrate nucleic acid molecule. After a round of addition, the nucleic acid substrate molecule is separated from the enzyme and nucleoside triphosphates and the chemical blocking group is removed by a treatment that leaves the rest of the substrate nucleic acid molecule unchanged. The 3′ hydroxyl is exposed during this deblocking step, readying the substrate nucleic acid molecule for another addition cycle. This strategy is illustrated in FIG. 1A.

The EOS strategy described in this disclosure differs from the one described above using 3′-blocked nucleotides by using natural nucleotides that have unblocked or free 3′ hydroxyls. The addition of a single nucleotide per addition cycle in the present disclosure depends on specific qualities of the nucleic acid polymerase with TINAP activity that allows it to extend the substrate nucleic acid molecule with a single nucleotide per addition cycle. The EOS strategy described in the present disclosure is illustrated in FIG. 1C.

A nucleic acid synthesis process based on the strategy described in this disclosure minimally involves combining a substrate nucleic acid molecule, a nucleic acid polymerase (TINAP) and one or more nucleoside triphosphates in a reaction mixture suitable for polymerase activity (minimally containing a buffering agent and a divalent cation at or close to physiological pH), allowing the reaction to proceed for sufficient time for the reaction to go to completion, then separating the substrate nucleic acid molecule, modified by the addition of a single nucleotide, from the nucleic acid polymerase and the unincorporated nucleoside triphosphates, and repeating the cycle.

The present disclosure includes use of any unblocked nucleoside triphosphate for synthesizing nucleic acids. The nucleoside triphosphate can be a ribonucleoside triphosphate such as ATP, CTP, GTP, ITP, UTP or XTP or any modified forms thereof, used for synthesizing RNA or modified forms of RNA. The nucleoside triphosphate can be a deoxyribonucleoside triphosphate such as dATP, dCTP, dGTP, dITP, dUTP or dXTP or any modified forms thereof, used for synthesizing DNA or modified forms of DNA.

Modified forms of nucleotides include, but are not limited to, nucleotides modified by covalent addition of methyl groups, O-methyl groups, hydroxyl groups, amino groups, phosphates, chlorine or fluorine atoms, mono-, di-or poly-saccharides, dyes, fluorescent groups, phosphorothioate groups (substituting the oxygen atoms on the phosphodiester linkage with sulfur atoms), binding groups (such as biotin or digoxygenin), reactive groups such as azides, aldchydes, ketones, thiols, disulfides or amines, or molecules containing one or more of the above. Modifying groups can be added to the nitrogenous bases of a nucleotide or the 2′ or 5′ carbons of the ribose sugar (for example 2′-fluoro or 2′-O-methyl substitutions), but can modify any carbon, nitrogen or oxygen atom found in the nucleotide, with the exception of the 3′-hydroxyl group. Multiple modifying groups can be added to a single nucleotide molecule. The purpose of modifying groups added to nucleotides is to allow specific detection, purification, targeting (to a tissue or cell type in an organism) or stabilization of a molecule to which the modified nucleotide has been covalently added, or combinations thereof.

The present disclosure can be used to synthesize any nucleic acid molecule of any sequence. The synthesized nucleic acid molecule can be DNA or RNA or modified forms thereof, or chimeric nucleic acids containing both ribonucleotides and deoxyribonucleotides or modified forms thereof. The synthesized sequence can contain canonical ribose or deoxyribose backbones or modified forms thereof, with any of a number of modifications to the ribose sugars, including but not limited to 2′-fluoro or 2′-O-methyl substitutions. The synthesized sequence can contain any of the canonical bases found in DNA and RNA (adenine, cytidine, guanine, thymine, uracil) or uncommon bases (for example hypoxanthine, xanthine) or modified forms of any such bases, or any mixtures of natural or modified bases. Modified forms of nitrogenous bases include but are not limited to bases modified by covalent addition of methyl groups, O-methyl groups, hydroxyl groups, amino groups, phosphates, chlorine or fluorine atoms, mono-, di-or poly-saccharides, dyes, fluorescent groups, phosphorothioate groups (substituting the phosphates), binding groups (such as biotin or digoxygenin), reactive groups such as azides, aldehydes, ketones, thiols, disulfides or amines, or molecules containing one or more of the above.

The substrate nucleic acid molecule used as a nucleotide acceptor in an enzymatic nucleic acid synthesis reaction can be of any length or sequence. For example, the substrate nucleic acid molecule can be 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000 or 100000 nucleotides in length, or longer, or any length in between.

The substrate nucleic acid molecule used as a nucleotide acceptor in an enzymatic nucleic acid synthesis reaction can be free in solution or immobilized on a solid support such as agarose beads, polystyrene beads or magnetic beads. Immobilization of the substrate nucleic acid molecule can occur via a covalent bond to the solid support or by non-covalent association with a solid support.

The substrate nucleic acid molecule used as a nucleotide acceptor in an enzymatic nucleic acid synthesis reaction can be either single-stranded or partially single-stranded. The 3′ end of the substrate nucleic acid molecule that serves as the nucleotide acceptor will be single-stranded, meaning that it will not be base paired to a homologous nucleotide, but any nucleotide in the substrate nucleic acid molecule that lies 5′ of the 3′ end can be single-stranded or double stranded.

The substrate nucleic acid molecule used as a nucleotide acceptor in an enzymatic nucleic acid synthesis reaction can be of any length, including 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000 or 100000 nucleotides in length, or longer, or any length in between.

The substrate nucleic acid molecule used as a nucleotide acceptor in an enzymatic nucleic acid synthesis reaction can contain deoxyribonucleotide residues or ribonucleotide residues, or a mixture of both deoxyribonucleotide and ribonucleotide residues. The nucleotide residues in the substrate nucleic acid molecule can contain any modifications, including modifications to the ribose sugars, or modifications to the bases, or modifications to the backbone.

The substrate nucleic acid molecule used as a nucleotide acceptor in an enzymatic nucleic acid synthesis reaction can be a pure molecule of a specific sequence and structure or can be a mixed population of different sequences or structures.

The nucleic acid sequence synthesized using the compositions and methods described in the present disclosure can contain all bases commonly found in the synthesized type of nucleic acid (i.c. A, C, G and T in the case of DNA) or a subset of these bases. The synthesized sequence may be complex or non-repetitive, or may be repetitive, with one or more specific sequences recurring. The synthesized sequence may be homopolymeric (containing only a single nucleotide) or may contain simple repeats of 2 or more nucleotides per repeat length, or complex repeats of 5 or more nucleotides in length.

The nucleic acid molecules synthesized using the compositions and methods described in the present disclosure can be of any length 2 nucleotides or longer, including 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000., 70000, 80000, 90000 or 100000 nucleotides or longer, or any length in between.

The efficiency of nucleotide addition when synthesizing nucleic acids using the compositions and methods described in the present disclosure can range from 1% to 100%. This means that during a single addition cycle, only a subset of the nucleic acid substrate molecules may be extended by an additional nucleotide by the nucleic acid polymerase. For example, the addition efficiency for any specific nucleotide to any specific nucleic acid substrate molecule can be 1%, 2%, 3%, 4%, 5%, 6%, 7%, 8%, 9%, 10%, 115, 12%, 13%, 14%, 15%, 16%, 17%, 18%, 19%, 20%, 30%, 40%, 50%, 60%, 70%, 80%, 90% or 100% or any percentage in between.

The efficiency of nucleotide addition by a nucleic acid polymerase can be influenced by a number of factors or variables in the reaction, including but not limited to the concentration of their respective nucleoside triphosphates present in the addition reaction, enzyme concentrations, and reaction conditions influencing enzyme activity. For example, raising the concentration of a specific nucleoside triphosphate can increase the incorporation efficiency of that nucleoside triphosphate. Similarly, increasing the concentration of an enzyme catalyzing the incorporation of a specific nucleoside triphosphate can increase the incorporation frequency of the nucleoside triphosphate. The same can be accomplished by altering the reaction mixture and reaction conditions, for example by varying the presence of buffering agents (for example Tris, sodium or potassium phosphate, sodium or potassium acetate or sodium or potassium cacodylate), salts, divalent cations and reaction additives or stabilizing agents including but not limited to polyethylene glycol, polyvinylpyrrolidone, glycerol, polyamines, detergents, surfactants, bovine serum albumin, DNA-binding proteins, formamide or molecules that affect or modify the nucleic acid polymerase activity such as peptides or small molecules; or by varying the concentration(s) of buffering agents, salts, divalent cations, nucleoside triphosphates and other reaction components including but not limited to polyethylene glycol, polyvinylpyrrolidone, glycerol, polyamines, detergents, surfactants, bovine serum albumin, DNA-binding proteins, formamide or molecules that affect or modify the nucleic acid polymerase activity such as peptides or small molecules.

The reaction pH of a nucleic synthesis process can vary around physiological pH by several pH units, for example pH 4.0, 5.0, 6.0, 7.0, 8.0, 9.0 or 10.0 or any pH in between.

Based on known mechanisms of nucleotide addition by nucleic acid polymerases, there are various possible mechanisms by which a TINAP can catalyze the addition of a single nucleotide to the 3′ end of an unblocked nucleic acid without undergoing processive addition of multiple nucleotides. These include, but are not limited to, the following. 1) A nucleic acid polymerase may be specific for a specific nucleic acid sequence, including the terminal bases on a nucleic acid substrate, and only add a nucleotide to substrate molecules containing this specific sequence. Once a nucleotide has been added, the end sequence is different and the polymerase may not be able to add another nucleotide to the substrate. 2) A nucleic acid polymerase may be defective in the translocation step of its nucleotide addition mechanism, which would stall the enzyme after the catalytic step of nucleotide addition and release of pyrophosphate, allowing the polymerase to add only a single nucleotide. 3) A nucleic acid polymerase may remain tightly associated in a covalent or non-covalent manner with the end of a nucleic acid molecule, preventing dissociation of the polymerase after nucleotide addition, and preventing access to the 3′ end of the nucleic acid by another molecule of the polymerase. 4) A nucleic acid polymerase may lose catalytic activity after addition of a single nucleotide rendering it incapable of adding additional nucleotides. These mechanisms and enzyme qualities may be present individually or in combination in specific nucleic acid polymerases.

Nucleic acid polymerases that exhibit sequence specificity in their addition of nucleotides to the 3′ end of a nucleic acid (the first mechanism of single-nucleotide addition listed above) can recognize and be specific for different numbers of nucleotides located in different parts of the nucleic acid. For example, a nucleic acid polymerase may be specific to the sequence present at the 3′ end of a nucleic acid or to an internal sequence that does not include the nucleotide present at the 3′ end. The polymerase may be specific to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides present at the 3′ end of the nucleic acid or internally. When recognizing a specific sequence internal to the nucleic acid, the distance from the 3′ end of the nucleic acid can be of different lengths, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides from the 3′ end of the nucleic acid. The recognition sequence governing sequence specificity of a nucleic acid polymerase may also reside in more than one non-contiguous sequence within the nucleic acid.

A nucleic acid polymerase that loses catalytic activity after addition of a single nucleotide to the 3′ end of a nucleic acid can do so in a reversible or irreversible manner. If reversible then there are treatments such as pH change; changes in the concentrations of salts, divalent cations, pyrophosphate, nucleoside monophosphates, nucleoside diphosphates, nucleoside triphosphates, reducing agents, or combinations of any of the preceding; changes in polymerase concentration; treatment with chaotropic agents such as guanidine, urca or alcohols; partial or complete unfolding followed by refolding or any other treatment known to those skilled in the art that restore the activity of the polymerase. These treatments will not restore polymerase activity if the loss of activity is irreversible.

A nucleic acid polymerase employed in an industrial nucleic acid synthesis process can be used once and then discarded or can be recycled in between nucleotide addition cycles for continued use. A nucleic acid polymerase may be used for any number of nucleotide addition cycles, for example for 1, 2, 3, 4, 5, 6, 7, 8, 9, 10, 11, 12, 13, 14, 15, 16, 17, 18, 19, 20, 30, 40, 50, 60, 70, 80, 90, 100 cycles or any number in between. Between cycles, a nucleic acid polymerase can be desalted, concentrated or separated from the other reaction components by any of a number of protein purification methods, including but not limited to affinity chromatography, anion exchange chromatography, cation exchange chromatography, gel filtration chromatography, reversed-phase chromatography or ultrafiltration, to prepare it for the next nucleotide addition cycle.

In between nucleotide addition cycles, a nucleic acid polymerase employed in an industrial nucleic acid synthesis process can be partially or completely unfolded or denatured (meaning to partly or fully transition the protein from its characteristic three-dimensional structure to a random coil) and refolded to its native 3-dimensional structure to prepare it for the next nucleotide addition cycle.

A single-nucleotide addition reaction may employ different stoichiometries of substrate to enzyme, falling into three genera categories: 1) Molar excess of enzyme; 2) Equimolar amounts of enzyme and substrate ends and 3) Molar excess of nucleic acid substrate 3′ ends. In the case of a molar excess of enzyme, the enzyme may be present at concentrations representing a fold excess compared to the concentration of the nucleic acid substrate 3′ ends, for example, 1.01×, 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20, 30×, 40×, 50×60×, 70×, 80×, 90×, 100×, or any number/fold excess in between. In the case of a molar excess of nucleic acid substrate 3′ ends, the nucleic acid substrate or the 3′ ends of a substrate (for example in the case of a covalently immobilized substrate) may be present at concentrations representing a fold excess compared to the concentration of the enzyme, for example, 1.01×, 1.1×, 1.2×, 1.3×, 1.4×, 1.5×, 1.6×, 1.7×, 1.8×, 1.9×, 2×, 3×, 4×, 5×, 6×, 7×, 8×, 9×, 10×, 20, 30×, 40×, 50×, 60×, 70×, 80×, 90×, 100×, 200×, 300×, 400×, 500×, 600×, 700×, 800×, 900×, 1000×, or any number/fold excess in between.

The ability to synthesize nucleic acids by controlled addition of single nucleotides can be exploited to create an industrial process for nucleic acid synthesis. Such an industrial process typically includes a specific composition of materials associated with the nucleic acid being synthesized, either in solution or on a solid support, specialized containers or vessels in which the synthesis takes place (for example flow columns), specific techniques for adding and removing enzymes and nucleoside triphosphates (for example involving specialized delivery systems or microfluidics), specific techniques for removing excess enzymes and nucleoside triphosphates after each nucleotide addition step, and specific methods of removing the enzyme from the reaction vessel after synthesis and separating it from the materials present during the synthesis such as a solid support, buffering agents, salts and other solutes.

An industrial process for nucleic acid synthesis can be developed at different reaction temperatures, for example 0, 1, 2, 3, 4, 5, 6, 7, 8, 9, 10, 11, 12, 13, 14, 15, 16, 17, 18, 19, 20, 30, 40, 50, 60, 70 80, 90, 100, 110, or 120 degrees Celsius or any temperature in between. The reaction temperature can be constant or can vary in the course of the reaction in any manner, for example by linear or nonlinear increases from a starting temperature, or linear or nonlinear decreases from a starting temperature, or by cyclical temperature changes, or any combinations thereof.

An industrial nucleic acid synthesis process can use different reaction times for each nucleotide addition cycle, for example 1, 2, 3, 4, 5, 6, 7, 8, 9, 10, 11, 12, 13, 14, 15, 16, 17, 18, 19, 20, 30, 40, 50 or 60 seconds per cycle or any time in between, or 1, 2, 3, 4, 5, 6, 7, 8, 9, 10, 11, 12, 13, 14, 15, 16, 17, 18, 19, 20, 30, 40, 50 or 60 minutes per cycle or any time in between, or 1, 2, 3, 4, 5, 6, 7, 8, 9, 10, 11, 12, 13, 14, 15, 16, 17, 18, 19, 20,, 21, 22, 23 or 24 hours per cycle, or any time in between.

An industrial process for nucleic acid synthesis can be set up at various scales to allow efficient synthesis of different quantities of nucleic acid. The scale can vary from fmol quantities of nucleic acid synthesized to mole quantities or higher. For example, specific processes can be devised for the synthesis of 1×10⁻¹⁶, 2×10⁻¹⁶, 3×10⁻¹⁶, 4×10⁻¹⁶, 5×10⁻¹⁶, 6×10⁻¹⁶, 7×10⁻¹⁶, 8×10⁻¹⁶, 9×10⁻¹⁶, 1×10⁻¹⁵, 2×10⁻¹⁵, 3×10⁻¹⁵, 4×10⁻¹⁵, 5×10⁻¹⁵, 6×10⁻¹⁵, 7×10⁻¹⁵, 8×10⁻¹⁵, 9×10⁻¹⁵, 1×10⁻¹⁴, 2×10⁻¹⁴, 3×10⁻¹⁴, 4×10⁻¹⁴, 5×10⁻¹⁴, 6×10⁻¹⁴, 7×10⁻¹⁴, 8×10⁻¹⁴, 9×10⁻¹⁴, 1×10⁻¹³, 2×10⁻¹³, 3×10⁻¹³, 4×10⁻¹³, 5×10⁻¹³, 6×10⁻¹³, 7×10⁻¹³, 8×10⁻¹³, 9×10⁻¹³, 1×10⁻¹², 2×10⁻¹², 3×10⁻¹², 4×10⁻¹², 5×10⁻¹², 6×10⁻¹², 7×10⁻¹², 8×10⁻¹², 9×10⁻¹², 1×10⁻¹¹, 2×10⁻¹¹, 3×10⁻¹¹, 4×10⁻¹¹, 5×10⁻¹¹, 6×10⁻¹¹, 7×10⁻¹¹, 8×10⁻¹¹, 9×10⁻¹¹, 1×10⁻¹⁰, 2×10⁻¹⁰, 3×10⁻¹⁰, 4×10⁻¹⁰, 5×10⁻¹⁰, 6×10⁻¹⁰, 7×10⁻¹⁰, 8×10⁻¹⁰, 9×10⁻¹⁰, 1×10⁻⁹, 2×10⁻⁹, 3×10⁻⁹, 4×10⁻⁹, 5×10⁻⁹, 6×10⁻⁹, 7×10⁻⁹, 8×10⁻⁹, 9×10⁻⁹, 1×10⁻⁸, 2×10⁻⁸, 3×10⁻⁸, 4×10⁻⁸, 5×10⁻⁸, 6×10⁻⁸, 7×10⁻⁸, 8×10⁻⁸, 9×10⁻⁸, 1×10⁻⁷, 2×10⁻⁷, 3×10⁻⁷, 4×10⁻⁷, 5×10⁻⁷, 6×10⁻⁷, 7×10⁻⁷, 8×10⁻⁷, 9×10⁻⁷, 1×10⁻⁶, 2× 10⁻⁶, 3×10⁻⁶, 4×10⁻⁶, 5×10⁻⁶, 6×10⁻⁶, 7×10⁻⁶, 8×10⁻⁶, 9×10⁻⁶, 1×10⁻⁵, 2× 10⁻⁵, 3×10⁻⁵, 4×10⁻⁵, 5×10⁻⁵, 6×10⁻⁵, 7×10⁻⁵, 8×10⁻⁵, 9×10⁻⁵, 1×10⁻⁴, 2×10⁻⁴, 3×10⁴, 4×10⁻⁴, 5×10⁻⁴, 6×10⁻⁴, 7×10⁻⁴, 8×10^{31 4}, 9×10⁻⁴, 1×10⁻³, 2×10⁻³, 3×10⁻³, 4×10⁻³, 5×10⁻³, 6×10⁻³, 7×10⁻³, 8×10⁻³, 9×10⁻³, 1×10⁻², 2×10⁻², 3×10⁻², 4×10⁻², 5×10⁻², 6×10⁻², 7×10⁻², 8×10⁻², 9×10⁻², 1×10⁻¹, 2×10⁻¹, 3×10⁻¹, 4×10⁻¹, 5×10⁻¹, 6×10⁻¹, 7×10⁻¹, 8×10⁻¹, 9×10⁻¹, 1, 2, 3, 4, 5, 6, 7, 8, 9, 10, 11, 12, 13, 14, 15, 16, 17, 18, 19, 20, 30, 40, 50, 60, 70 80, 90 or 100 moles of nucleic acid, or any scale in between.

An industrial process for nucleic acid synthesis can rely either on a single enzyme that has all the required activities for addition of any nucleotide with any structure to the 3′ end of any nucleic acid, or the process may rely on specialized enzymes to catalyze the addition of specific nucleotides to specific nucleic acids. For example, a nucleic acid polymerase used for addition of a ribonucleotide may differ from the nucleic acid polymerase used to add a deoxyribonucleotide. Different nucleic acid polymerases may be used to add nucleotides containing different bases or different modifications. Different nucleic acid polymerases may be used to add nucleotides to nucleic acids differing in the sequences present at the nucleic acids' 3′ end or sequences present internal to the nucleic acid. Different nucleic acid polymerases may be used to add nucleotides with different linkages, for example canonical phosphodiester linkages compared to phosphorothioate linkages. An industrial process may use 1, 2, 3, 4, 5, 6, 7, 8, 9, 10, 11, 12, 13, 14, 15, 16, 17, 18, 19, 20, 30, 40, 50, 60, 70 80, 90, 100, 200, 300, 400, 500, 600, 700, 800, 900 or 1000 different nucleic acid polymerases, or any number in between, to allow synthesis of different sequences and/or structures of nucleic acids.

For each cycle in a nucleic acid synthesis, a nucleic acid polymerase will be added to catalyze the specific addition reaction required for this cycle. The nucleic acid polymerase can be a single enzyme or a mixture of 2 or more enzymes.

Enzymatic oligonucleotide synthesis can allow incorporation of degenerate or mixed nucleotides at specific positions in an oligonucleotide. This involves adding multiple nucleoside triphosphates into the enzymatic addition reaction for a specific addition cycle. Depending on the structure of the nucleotides to be incorporated into the mixed position, one or more nucleic acid polymerases are added to catalyze the incorporation reactions.

When synthesizing nucleic acids with degenerate or mixed nucleotides in a specific position, multiple enzymes can be added to allow addition of multiple nucleotides to a single position in the nucleic acid in a specific addition cycle.

The ratio of incorporated nucleotides at a degenerate position can be influenced by the concentration of their respective nucleoside triphosphates present in the addition reaction, enzyme concentrations, and reaction conditions influencing relative rates of different enzymes. For example, raising the concentration of a specific nucleoside triphosphate within a mixture of two or more nucleoside triphosphates will typically increase the incorporation efficiency of that nucleoside triphosphate. Similarly, increasing the concentration of an enzyme catalyzing the incorporation of a specific nucleoside triphosphate within a mixture will increase the incorporation frequency of that nucleoside triphosphate. The same can be accomplished by altering reaction conditions (presence of buffering agents, salts, divalent cations and reaction additives or stabilizing agents including but not limited to polyethylene glycol, polyvinylpyrrolidone, glycerol, polyamines, detergents, bovine serum albumin, DNA-binding proteins or formamide; concentration of buffering agents, salts, divalent cations, nucleoside triphosphates and other reaction components including but not limited to polyethylene glycol, polyvinylpyrrolidone, glycerol, polyamines, detergents, bovine serum albumin, DNA-binding proteins or formamide; pH; temperature) to optimize the activity of a nucleic acid polymerase, or favor the activity of one nucleic acid polymerase relative to other nucleic acid polymerases present in the mixture.

An oligonucleotide synthesized enzymatically can contain any number of degenerate nucleotides, including 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000., 70000, 80000, 90000 or 100000 or more degenerate nucleotides, up to the total length of the oligonucleotide. A degenerate position in the oligonucleotide can consist of a mixture of all four canonical nucleotides A, C, G and T, or a subset of bases (for example A+C, A+G, A+T, C+G, C+T, G+T, A+C+G, A+C+T, A+G+T, C+G+T) or any mixture of canonical nucleotides with non-natural or modified nucleotides of any kind.

In an enzymatic nucleic acid synthesis process, the nucleic acid being synthesized can be either in solution or coupled to a solid support, or a combination thereof. When using a solid support, the nucleic acid can be covalently attached to the solid support or non-covalently attached.

Different solid supports can be used to immobilize a nucleic acid during synthesis and are known to those trained in the art. These include, but are not limited to, controlled pore glass (CPG) beads, agarose beads or resins, polystyrene beads or resins, PEG beads or resins, silica gel beads and a number of other specialized materials developed for immobilization of chemical groups, enzymes or nucleic acids. Solid supports can have a variety of bead sizes ranging from 0.01-1000 microns and pore sizes ranging from 0.01-1000 microns.

The nucleic acid polymerase used in an enzymatic nucleic acid synthesis reaction can be free in solution or immobilized on a solid support including but not limited to agarose beads, polystyrene beads or magnetic beads. Immobilization of the nucleic acid polymerase can occur via a covalent bond to the solid support or by non-covalent association with a solid support. The solid support used to immobilize the nucleic acid polymerase can be the same solid support used to immobilize the nucleic acid substrate, or can be a different support.

The nucleic acid polymerase used in an enzymatic nucleic acid synthesis reaction can be a DNA polymerase or an RNA polymerase based on its natural function. In the case of DNA polymerases, the polymerase can belong to any of different known families of DNA polymerases, including but not limited to families A, B, C, D, X, Y and RT.

The nucleic acid polymerase used in an enzymatic nucleic acid synthesis reaction can be a natural enzyme or an engineered enzyme meaning that its sequence or structure has been altered by the hand of man to increase its utility for de novo nucleic acid synthesis.

This disclosure describes seven novel nucleic acid polymerases capable of adding single nucleotides to the 3′ end of a nucleic acid molecule. The SEQ ID NOs for these enzymes are given in Table 1 below, and their activities are described in Example 1.

TABLE 1

| Nucleic acid polymerases | | | | | | | |
|---|---|---|---|---|---|---|---|
| Enzyme | Accession | Plasmid | | SEQ ID NOS: | | | |
| name | number | name | Species | A | B | C | D |
| EDS017 | Q04049 | PP1077 | Saccharomyces | 1 | 11 | 21 | 31 |
| | | | cerevisiae | | | | |
| EDS024 | BAD02935 | PP1084 | Takifugu rubripes | 2 | 12 | 22 | 32 |
| EDS029 | KTA96827.1 | PP1089 | Candida glabrata | 3 | 13 | 23 | 33 |
| EDS030 | XP_011273936.1 | PP1090 | Wickerhamomyces | 4 | 14 | 24 | 34 |
| | | | ciferrii | | | | |
| EDS053 | AYW42506.1 | PP1113 | Pseudomonas | 5 | 15 | 25 | 35 |
| | | | aeruginosa | | | | |
| EDS054 | WP_124690524.1 | PP1114 | Pigmentiphaga sp. | 6 | 16 | 26 | 36 |
| | | | H8 | | | | |
| EDS066 | XP_031753771.1 | PP1126 | Xenopus tropicalis | 7 | 17 | 27 | 37 |
| EDS082 | XP_011273936.1 | PP1142 | Wickerhamomyces | 8 | 18 | 28 | 38 |
| | | | ciferrii | | | | |
| EDS048 | DAA14763.1 | PP1108 | Bos taurus | 9 | 19 | 29 | 39 |
| EDS015 | NP_001036693 | PP1075 | Mus musculus | 10 | 20 | 30 | 40 |

Wherein the SEQ ID NOs in column A are natural sequences (amino acid)
the SEQ ID NOs in column B are the cloned gene sequences (nucleic acid)
the SEQ ID NOs in column C are the expressed protein sequences (amino acid)
the SEQ ID NOs in column D are expression plasmid sequences (nucleic acid)

As noted above, nucleic acid polymerases can have a partial ability to add single nucleotides to the 3′ end of a nucleic acid substrate, meaning that the addition efficiency of single nucleotides to a nucleic acid substrate during a reaction may be less than 100%. In order to raise this efficiency, nucleic acid polymerases can be engineered to be more efficient. This means that variants of the original enzyme are produced that have a higher addition efficiency in a reaction than the parental enzyme. The nucleic acid polymerase can also be engineered to alter its substrate specificity. For example, a nucleic acid polymerase that efficiently adds nucleotides to the 3′ end of a nucleic acid ending in T can be engineered to efficiently add nucleotides to nucleic acids ending in any nucleotide. As another example, a nucleic acid polymerase that efficiently adds A to the 3′ end of a nucleic acid may be engineered for broader substrate specificity, so that variant enzymes are able to efficiently add any nucleotide to the 3′ end of a nucleic acid molecule. In yet another example, a nucleic acid polymerase that in a processive manner adds multiple nucleotides to the 3′ end of a nucleic acid in a reaction can be engineered to add only single nucleotides to the 3′ end during the reaction. In a further example, a nucleic acid polymerase that in efficiently adds deoxyribose nucleotides to the 3′ end of a nucleic acid can be engineered to efficiently add ribonucleotides. In a further example, a nucleic acid polymerase that in efficiently adds deoxyribose nucleotides to the 3′ end of a DNA molecule can be engineered to efficiently add deoxyribonucleotides to an RNA molecule. In a final example, a nucleic acid polymerase that in efficiently adds ribonucleotides to the 3′ end of a DNA molecule can be engineered to efficiently add ribonucleotides to the 3′ end of an RNA molecule. These examples are not exhaustive, and in practice it is possible to engineer any specific desirable nucleic acid polymerase activity by engineering a starting enzyme that either lacks this activity or exhibits this activity with low efficiency.

Many approaches and methods for protein engineering have been described in the literature, including but not limited to those listed in the following review articles: Leatherbarrow 1986, Zoller 1991, Lutz 2000, Leisola 2007, Eisenbeis 2010, O'Fágáin 2011, Foo 2012, Zawaira 2012, Marcheschi 2013, Woodley 2013, Johnson 2014, Packer 2015, Shin 2015, Chen 2016, Kaushik 2016, Swint-Kruse 2016, Wrenbeck 2017, Bornscheuer 2018, Lutz 2018, Singh 2018, Sinha 2019, Wilding 2019, Yang 2019.

In general, protein engineering uses one or more methods to diversify the gene sequence encoding an enzyme of interest, followed by one or more selection or screening methods used to select genes that encode variant enzymes improved in one or more qualities of interest. Qualities of interest include but are not limited to: nucleotide addition efficiency in specific reaction conditions or when modifying specific substrates; substrate specificity relating to the nucleic acid substrate; resistance to inhibitors; substrate specificity relating to the nucleoside triphosphate; stability when exposed to high temperature; stability under conditions that may inactivate a parental enzyme such as presence in the reaction of salts, pyrophosphate or other reaction products, or any other chemical or compound; high concentrations in the reaction of any of the aforementioned; or any other quality of the enzyme that may improve its suitability for an enzymatic nucleic acid synthesis process.

Methods for diversifying a gene encoding a nucleic acid polymerase of interest include, but are not limited to: mutagenesis meaning introduction of point mutations; introduction of insertions and deletions of varying lengths within the enzyme coding sequence; fusion with other sequences either at the 5′ or the 3′ end of the coding sequence; homologous sequence exchange with related coding sequences resulting in reassortment of polymorphisms; and any other means of creating sequence diversity.

A subset of template-independent nucleic acid polymerases contain a BRCT domain which is not essential for nucleic acid polymerase activity and which may mediate interactions with other proteins involved in DNA synthesis or repair (Callebaut 1997, Repasky 2004). Truncation of the protein to remove the BRCT domain has been reported to stimulate DNA polymerase activity in terminal deoxynucleotidyltransferases (Mueller 2009). Similar targeted truncations that remove the BRCT domain may be used to alter the activity of other TINAPs.

Methods and approaches used to select for genes encoding enzymes improved in one or more qualities of interest include approaches using in vitro compartmentalization in microdroplets or emulsions that allow efficient processing of high numbers of enzyme variants in small volumes. Such approaches have been described in the literature in a general manner and in specific applications to nucleic acid processing enzymes (Tawfik 1998, Ghadessy 2001, Dichl 2006, Griffiths 2006, Miller 2006, Ghadessy 2007, Tay 2010, Takeuchi 2014).

### EXAMPLES

### Example 1: Single Nucleotide Addition to Oligonucleotides in Solution DNA Polymerases, Enzyme Expression and Purification

Genes encoding the DNA polymerases listed in Table 1, each with a six-histidine tag at their N-terminus (SEQ ID NOs: 21-30) are designed as nucleic acid sequences (SEQ ID NOs: 11-20), synthesized by commercial gene synthesis supplier and cloned into a bacterial expression plasmid with an MB1 plasmid replicon conferring a high copy number in E. coli. The insertion site for the DNA polymerase genes on the plasmid is flanked by an arabinose inducible promoter and a Lambda T1 terminator, allowing for arabinose-inducible expression of each polymerase. The expression construct is sequence verified after cloning. The full sequence of the expression constructs for the DNA polymerases covered in this disclosure is given in SEQ ID NOs: 31-40.

The coding sequence of the gene encoding EDS082 was obtained by truncating the sequence coding for EDS030. The sequence encoding the BRCT domain present at the N-terminus of EDS030 was removed as has been described for other polymerases (Mueller 2009) and a methionine codon inserted at the start of the shortened coding sequence.

The expression plasmid is transformed into the E. coli strain BL21 and a single colony picked for cultivation and protein expression. The bacterial cells are grown in LB medium at 37°° C. to log phase culture and induced by addition of L-arabinose. After 18 hours of incubation at 15° C., the cultures are harvested by centrifugation and the collected E. coli cells are lysed. DNA polymerase is purified with nickel affinity chromatography according to manufacturer's instructions. The DNA polymerase is eluted with imidazole solution, concentrated with AMICON® Ultra-centrifugal filter sold by Millipore (Darmstadt, Germany) and changed into a storage buffer composed of 50 mM KPO4, pH7.3, 100 mM NaCl, 1.43 mM Beta mercaptocthanol, 0.05% Triton-X100, and 50% glycerol.

### In Vitro Nucleotide Addition Assay With Oligonucleotide and dNTP Pools

Enzyme activity is assayed by performing reactions in a buffer composed of 50 mM potassium acetate and 20 mM Tris acetate at pH 7.5. Reaction buffer is supplemented with 10 mM magnesium acetate and 250 μM cobalt chloride. Reactions are performed in the presence of 500 μM dNTPs, 10 μM of single stranded DNA oligonucleotide and 1 μg of enzyme/10 μl reaction. Reactions are incubated using a temperature gradient starting at 15° C. and ramping up to 50° C. at a rate of 1° C./min. Reactions are performed in 10 μl volumes and set up on ice.

For activity screening, an equimolar mixture of single stranded DNA oligonucleotides is used: PG5861 (GTCCTCAATCGCACTGGAAT, SEQ ID NO: 45); PG5859 (GTCCTCAATCGCACTGGAAG, SEQ ID NO: 43); PG5860 (GTCCTCAATCGCACTGGAAC, SEQ ID NO: 44); PG5858 (GTCCTCAATCGCACTGGAAA, SEQ ID NO: 42). The mix of single-stranded oligonucleotides is combined with an equimolar mixture of dATP, dTTP, dGTP, and dCTP. Oligonucleotides are synthesized by Eurofins Genomics (Louisville, KY) and dNTPs are purchased from New England Biolabs (Beverly, MA).

Reactions are stopped by addition of an equal volume of 2×NOVEX™ TBE-Urea Sample Buffer (ThermoFisher, Waltham, MA) and heated at 70° C. for 3 minutes. Samples are cooled and 15 μl added to a NOVEX™ TBE-Urea polyacrylamide gel (15%, ThermoFisher, Waltham, MA), electrophoresed at 150V, stained with methylene blue, destained with deionized water and imaged with white light using an AZURE™ 200 gel imaging workstation (Azure Biosystems, Dublin, CA).

An example of evaluation of the activity of 10 DNA polymerases is shown in FIG. 2. Various enzymes show a tendency to add one or several nucleotides to a single-stranded oligonucleotide, which may indicate suitability for an enzymatic nucleic acid synthesis process.

### Assay for Single Nucleotide Additions by Gel Electrophoresis

Enzyme activity using individual dNTPs is assayed by performing reactions in a buffer composed of 50 mM potassium acetate and 20 mM tris acetate at pH 7.5. Reaction buffer is supplemented with 10 mM magnesium acetate and 250 μM cobalt chloride. Reactions are performed in the presence of 500 μM dNTPs, 10 μM of single stranded DNA oligonucleotide and 1 μg of enzyme/10 μl reaction. Reactions are incubated at 30° C. for 15 minutes. Reactions were performed in 10 μl volumes and set up on ice.

The following individual dNTP and DNA oligonucleotide pairs are used for each reaction: dTTP+PG5861 (GTCCTCAATCGCACTGGAAT, SEQ ID NO: 45); dGTP+PG5864 (GTCCTCAATCGCACTGGAATT, SEQ ID NO: 46); dATP+PG5865 (GTCCTCAATCGCACTGGAATTG, SEQ ID NO: 47); dCTP+PG5866 (GTCCTCAATCGCACTGGAATTGA, SEQ ID NO: 48). A standard oligonucleotide is also used in the analysis: PG5867 (GTCCTCAATCGCACTGGAATTGAC, SEQ ID NO: 54).

Reactions are stopped by addition of an equal volume of 2×NOVEX™ TBE-Urca Sample Buffer (ThermoFisher, Waltham, MA) and heated at 70° C. for 3 minutes. Samples are cooled and 15 μl added to a NOVEX™ TBE-Urea polyacrylamide gel (15%, ThermoFisher, Waltham, MA), electrophoresed at 150V, stained with methylene blue, destained with deionized water and imaged with white light using an AZURE™ 200 gel imaging workstation (Azure Biosystems, Dublin, CA).

FIG. 3A shows efficient addition of single nucleotides to the four different oligonucleotide substrates listed above.

### Assay for Sequential Nucleotide Additions

Sequential nucleotide addition reactions are performed in a buffer composed of 50 mM potassium acetate and 20 mM tris acetate at pH 7.5. Reaction buffer was supplemented with 10 mM magnesium acetate and 250 μM cobalt chloride. Reactions are performed in the presence of 500 μM dNTPs, 10 μM of single stranded DNA oligonucleotide and I ug of enzyme/10 μl reaction. Reactions are incubated at 30° C. for 15 minutes. Reaction volumes are scaled up to as high as 100 μl when performing sequential reactions for addition of multiple dNTPs. The initial reaction is performed using a single stranded DNA oligonucleotide with the following sequence PG5861 (GTCCTCAATCGCACTGGAAT, SEQ ID NO: 45) and dTTP as the nucleoside triphosphate.

Reactions are stopped by boiling at 100° C. for 3 minutes and the oligonucleotide purified from reaction components on a silica column using the Oligonucleotide Clean and Concentrator kit from Zymo Research (Irvine, CA) according to the manufacturer's instructions and cluted in distilled water. The concentration of the purified oligonucleotide is measured using a NANODROPTM One spectrophotometer from Thermo Scientific (Waltham, MA) and an aliquot set aside for gel electrophoresis. The remaining purified oligonucleotide is then used in an additional reaction using dGTP in the same process as the starting oligonucleotide.

The following oligonucleotides are used as standards by adding to the sample and running duplicate analyses (see FIGS. 4B, D, F and H): PG5861 (GTCCTCAATCGCACTGGAAT, SEQ ID NO: 45); PG5864 (GTCCTCAATCGCACTGGAATT, SEQ ID NO: 46); PG5865 (GTCCTCAATCGCACTGGAATTG, SEQ ID NO: 47); PG5866 (GTCCTCAATCGCACTGGAATTGA, SEQ ID NO: 48); and PG5867 (GTCCTCAATCGCACTGGAATTGAC, SEQ ID NO: 54).

For analysis by gel electrophoresis, samples are diluted by addition of an equal volume of 2×NOVEX™ TBE-Urea Sample Buffer (ThermoFisher, Waltham, MA) and heated at 70° C. for 3 minutes. Samples are cooled and 15 μl added to a NOVEX™ TBE-Urea polyacrylamide gel (15%, ThermoFisher, Waltham, MA), electrophoresed at 150V, stained with methylene blue, destained with deionized water and imaged with white light using an AZURE™200 gel imaging workstation (Azure Biosystems, Dublin, CA).

FIG. 3B shows the efficient sequential addition of two nucleotides to the oligonucleotide substrate with the sequence given in SEQ ID NO: 45.

### Assay for Single Nucleotide Additions by Capillary Electrophoresis

Enzyme activity using individual dNTP oligonucleotide pairs are assayed by performing reactions in a buffer composed of 50 mM potassium acetate and 20 mM tris acetate at pH 7.5. Reaction buffer is supplemented with 10 mM magnesium acetate and 250 μM cobalt chloride. Reactions are performed in the presence of 500 uM dNTPs, 10 μM of single stranded DNA oligonucleotide and 1 μg of enzyme/10 μl reaction. Reactions are incubated at 30°° C. for 15 minutes. Reactions are performed in 10 μl volumes and set up on ice.

Oligonucleotides used: PG5861 (GTCCTCAATCGCACTGGAAT, SEQ ID NO: 45); PG5864 (GTCCTCAATCGCACTGGAATT, SEQ ID NO: 46); PG5872 (GTCCTCAATCGCACTGGAATG, SEQ ID NO: 53); PG5859 (GTCCTCAATCGCACTGGAAG, SEQ ID NO: 43); PG5868 (GTCCTCAATCGCACTGGAAGT, SEQ ID NO: 49); PG5869 (GTCCTCAATCGCACTGGAAGC, SEQ ID NO: 50); PG5858 (GTCCTCAATCGCACTGGAAA, SEQ ID NO: 42).

Enzymatic addition to each oligonucleotide is separately assessed with dATP, dTTP, dGTP, and dCTP in individual reactions. Reactions are stopped by boiling at 100° C. for 3 minutes and the oligonucleotide purified from reaction components on a silica column using the Oligonucleotide Clean and Concentrator kit from Zymo Research (Irvine, CA) according to the manufacturer's instructions and cluted in distilled water. Purified oligonucleotide is then analyzed on an Agilent Oligo Pro II capillary electrophoresis system by Agilent Technologies (Santa Clara, CA) using a 24-capillary array. Purified oligonucleotide in water is diluted to ˜0.5-2 μM for analysis using injection methods in the range of 9-12 kV for 10 seconds followed by separation at 15 kV for 70 minutes. Data is analyzed using Agilent Oligo Pro II Data Analysis Software 2.0.0.3 (Agilent Technologies, Santa Clara, CA). Analysis of the reactions is performed by running two independent runs for each sample. One run contains only pure sample on the Agilent Oligo Pro II to assess the purity and percent conversion of the starting oligonucleotide (FIGS. 4A, 4C, 4E and 4G). A second run is performed with standards spiked into each sample to accurately size the purified oligonucleotides after performing the reaction (FIGS. 4B, 4D, 4F and 4H).

The following oligonucleotide standards are spiked in at ˜1 μuM final concentration: PG1350 (GCGTCACGCTACCAACCA, SEQ ID NO: 41); PG5870 (GTCCTCAATCGCACTGGAAACATCAAGGTC, SEQ ID NO: 51); PG5871 (GTCCTCAATCGCACTGGAAACATCAAGGTCATACGGAACG, SEQ ID NO: 52). The oligonucleotide used in each specific reaction is also spiked in at ˜1 82 M together with the standards.

Profiles from representative capillary electrophoresis runs on the Agilent Oligo Pro Il instrument are shown in FIG. 4A-H. FIGS. 4A and 4B show capillary electrophoresis runs of control oligonucleotides not treated in enzymatic reactions. FIGS. 4C and 4D show partial addition of a single nucleotide to a single-stranded oligonucleotide after reaction of oligonucleotide PG5861 (SEQ ID NO: 45) with dTTP and enzyme EDS082 (see Table 1). FIGS. 4E and 4F show efficient addition of a single nucleotide to a single-stranded oligonucleotide after reaction of oligonucleotide PG5861 (SEQ ID NO: 45) with dTTP and enzyme EDS054 (see Table 1). FIGS. 4G and 4H show addition of 1, 2, 3, 4 and 5 nucleotides to a single-stranded oligonucleotide after reaction of oligonucleotide PG5861 (SEQ ID NO: 45) with dTTP and enzyme EDS066 (see Table 1).

The results of 50 representative reactions showing single-nucleotide addition are summarized in Table 2 below.

N signifies the length in nucleotides of the oligonucleotide that serves as a substrate in these reactions.

% <N means the percent of product that is shorter than N (for example degradation products of the oligonucleotide substrate).

% N means the percent of product that has a length of N (for example unreacted oligonucleotide substrate).

% N+1 means the percent of product that is one nucleotide longer than N (for example the desired extension product).

% N+>1 means the percent of product that is 2 or more nucleotides longer N (for example extension products of the oligonucleotide substrate that received two or more added nucleotides). The table clearly shows a yield of the desired N+1 extension product in each example, with single nucleotide addition efficiencies ranging from 36% to 100%.

TABLE 2
Results of 50 representative addition reactions

| | | Substrate | | | | | | |
|---|---|---|---|---|---|---|---|---|
| Reaction | Enzyme | (SEQ ID | | % < | % | % | % | |
| # | used | NO) | dNTP | N | N | N + 1 | N +> 1 | Total |
| 29 | EDS030 | 45 | G | 0% | 0% | 100% | 0% | 100% |
| 30 | EDS053 | 45 | G | 0% | 0% | 100% | 0% | 100% |
| 31 | EDS054 | 45 | G | 0% | 0% | 100% | 0% | 100% |
| 389 | EDS030 | 53 | A | 0% | 5% | 95% | 0% | 100% |
| 393 | EDS082 | 53 | A | 0% | 6% | 94% | 0% | 100% |
| 388 | EDS029 | 53 | A | 0% | 6% | 94% | 0% | 100% |
| 392 | EDS066 | 53 | A | 0% | 6% | 94% | 0% | 100% |
| 390 | EDS053 | 53 | A | 5% | 6% | 90% | 0% | 100% |
| 391 | EDS054 | 53 | A | 0% | 4% | 89% | 7% | 100% |
| 236 | EDS066 | 43 | C | 0% | 9% | 84% | 8% | 100% |
| 387 | EDS017 | 53 | A | 0% | 0% | 80% | 20% | 100% |
| 211 | EDS054 | 43 | T | 0% | 20% | 80% | 0% | 100% |
| 77 | EDS030 | 46 | G | 0% | 12% | 67% | 21% | 100% |
| 78 | EDS053 | 46 | G | 0% | 21% | 66% | 13% | 100% |
| 230 | EDS017 | 43 | C | 0% | 0% | 66% | 34% | 100% |
| 19 | EDS054 | 45 | T | 14% | 21% | 65% | 0% | 100% |
| 208 | EDS029 | 43 | T | 0% | 0% | 65% | 35% | 100% |
| 400 | EDS029 | 53 | T | 0% | 0% | 65% | 35% | 100% |
| 27 | EDS017 | 45 | G | 0% | 0% | 65% | 35% | 100% |
| 326 | EDS017 | 42 | C | 0% | 18% | 65% | 18% | 100% |
| 220 | EDS029 | 43 | G | 0% | 0% | 64% | 36% | 100% |
| 81 | EDS082 | 46 | G | 6% | 30% | 60% | 4% | 100% |
| 279 | EDS017 | 49 | C | 0% | 40% | 60% | 0% | 100% |
| 242 | EDS017 | 49 | A | 0% | 0% | 59% | 41% | 100% |
| 90 | EDS053 | 46 | C | 0% | 0% | 59% | 41% | 100% |
| 207 | EDS017 | 43 | T | 0% | 35% | 58% | 7% | 100% |
| 219 | EDS017 | 43 | G | 0% | 18% | 58% | 24% | 100% |
| 79 | EDS054 | 46 | G | 0% | 9% | 53% | 38% | 100% |
| 18 | EDS053 | 45 | T | 25% | 22% | 52% | 0% | 100% |
| 87 | EDS017 | 46 | C | 7% | 31% | 48% | 14% | 100% |
| 62 | EDS017 | 46 | T | 0% | 41% | 48% | 11% | 100% |
| 440 | EDS066 | 50 | A | 9% | 38% | 48% | 4% | 100% |
| 463 | EDS054 | 50 | G | 18% | 35% | 47% | 0% | 100% |
| 14 | EDS017 | 45 | T | 0% | 0% | 47% | 53% | 100% |
| 422 | EDS017 | 53 | C | 0% | 33% | 46% | 21% | 100% |
| 33 | EDS082 | 45 | G | 14% | 41% | 45% | 0% | 100% |
| 460 | EDS029 | 50 | G | 5% | 43% | 44% | 8% | 100% |
| 91 | EDS054 | 46 | C | 18% | 11% | 43% | 29% | 100% |
| 403 | EDS054 | 53 | T | 9% | 49% | 42% | 0% | 100% |
| 316 | EDS029 | 42 | G | 0% | 11% | 42% | 47% | 100% |
| 17 | EDS030 | 45 | T | 18% | 41% | 42% | 0% | 100% |
| 232 | EDS029 | 43 | C | 0% | 0% | 41% | 59% | 100% |
| 64 | EDS029 | 46 | T | 0% | 0% | 40% | 60% | 100% |
| 200 | EDS066 | 43 | A | 0% | 60% | 40% | 0% | 100% |
| 332 | EDS066 | 42 | C | 12% | 48% | 40% | 0% | 100% |
| 199 | EDS054 | 43 | A | 0% | 60% | 40% | 0% | 100% |
| 21 | EDS082 | 45 | T | 10% | 52% | 37% | 0% | 100% |
| 212 | EDS066 | 43 | T | 0% | 14% | 37% | 48% | 100% |
| 15 | EDS017 | 45 | T | 10% | 53% | 37% | 0% | 100% |
| 195 | EDS017 | 43 | A | 0% | 64% | 36% | 0% | 100% |

### Assay for Addition of Ribo-nucleotides

Enzyme activity using an equal molar mix of four NTPs is assayed by performing reactions in a buffer composed of 50 mM potassium acetate and 20 mM tris acetate at pH 7.5. Reaction buffer was supplemented with 10 mM magnesium acetate and 250 μM cobalt chloride. Reactions are performed in the presence of 500 μM NTPs, 10 μM of single stranded DNA oligonucleotide and 1 μg of enzyme/10 μl reaction. Reactions are incubated at a range of temperatures starting at 15° C. and ramping up to 37° C. at a rate of 1° C./minute. Reactions are performed in 10 μl volumes and set up on ice.

For initial activity screening (FIG. 5A), an equimolar mixture of single stranded DNA oligonucleotides is used: PG5861 (GTCCTCAATCGCACTGGAAT, SEQ ID NO: 45); PG5859 (GTCCTCAATCGCACTGGAAG, SEQ ID NO: 43); PG5860 (GTCCTCAATCGCACTGGAAC, SEQ ID NO: 44); PG5858 (GTCCTCAATCGCACTGGAAA, SEQ ID NO: 42). For assaying addition of NTP to a single stranded DNA oligonucleotide (FIG. 5B), PG5861 (GTCCTCAATCGCACTGGAAT, SEQ ID NO: 45) is used in each reaction.

Reactions are stopped by addition of an equal volume of 2×NOVEX™ TBE-Urea Sample Buffer (ThermoFisher, Waltham, MA) and heated to 70° C. for 3 minutes. Samples are cooled and 15 μl added to a NOVEX™ TBE-Urea polyacrylamide gel (15%, ThermoFisher, Waltham, MA), electrophoresed at 150V, stained with methylene blue, destained with water and imaged with white light using an AZURE™200 gel imaging workstation.

Examples of results from addition of ribonucleotides to DNA oligonucleotides are shown in FIG. 5. Enzymes EDS017, EDS024, EDS029, EDS030, EDS066, EDS082, EDS048 and EDS015 all showed the ability to incorporate ribonucleotides. In most cases, this incorporation was limited to 1-3 nucleotides.

The ability of different enzymes to add ribonucleotides to the ends of DNA oligonucleotides is summarized in Table 3.

TABLE 3
Summary of ribonucleotide addition to
DNA oligonucleotides by DNA polymerases

| | Maximal number of |
|---|---|
| Enzyme | ribonucleotides added |
| EDS017 | 2 |
| EDS024 | 2 |
| EDS029 | 1 |
| EDS030 | 10 |
| EDS053 | 0 |
| EDS054 | 0 |
| EDS066 | 2 |
| EDS082 | 4 |
| EDS048 | 3 |
| EDS015 | 2 |

### REFERENCES

Andrade P, Martín M J, Juárez R, López de Saro F, Blanco L (2009). Limited terminal transferase in human DNA polymerase mu defines the required balance between accuracy and efficiency in NHEJ. Proc Natl Acad Sci U S A 106 (38): 16203-16208.

Beard W A, Wilson S H (2014). Structure and mechanism of DNA polymerase beta. Biochemistry 53 (17): 2768-2780.

Bebenek K, Kunkel T A (2002) Family growth: the eukaryotic DNA polymerase revolution. Cell Mol Life Sci. 59 (1): 54-57.

Berdis A J (2009). Mechanisms of DNA polymerases. Chem Rev. 109 (7): 2862-2879.

Berdis A J (2014). DNA polymerases that perform template-independent DNA synthesis. Nucl. Acids Mol. Biol. 30:109-137.

Bornscheuer U T, Höhne M, Eds. (2018). Protein Engineering: Methods and Protocols. Methods Mol Biol. 1685. Humana Press, New York, NY.

Callebaut I, Mornon J P (1997). From BRCA1 to RAPI: a widespread BRCT module closely associated with DNA repair. FEBS Lett. 400 (1): 25-30.

Chang Y K, Huang Y P, Liu X X, Ko T P, Bessho Y, Kawano Y, Maestre-Reyna M, Wu W J, Tsai M D (2019). Human DNA Polymerase mu Can Use a Noncanonical Mechanism for Multiple Mn (2+)-Mediated Functions. J Am Chem Soc. 141 (21): 8489-8502.

Chen Z, Zeng A P (2016). Protein engineering approaches to chemical biotechnology. Curr Opin Biotechnol. 42:198-205.

Clark J M (1988). Novel non-templated nucleotide addition reactions catalyzed by procaryotic and cucaryotic DNA polymerases. Nucl Acids Res 16 (20): 9677-9686.

Dahl J M, Wang H, Lázaro J M, Salas M, Lieberman K R (2014). Dynamics of translocation and substrate binding in individual complexes formed with active site mutants of {phi} 29 DNA polymerase. J Biol Chem. 289 (10): 6350-6361.

Deibel M R Jr, Coleman M S (1980). Biochemical properties of purified human terminal deoxynucleotidyltransferase. J Biol Chem. 255 (9): 4206-4212.

Delarue M, Boulé JB, Lescar J, Expert-Bezançon N, Jourdan N, Sukumar N, Rougeon F, Papanicolaou C (2002). Crystal structures of a template-independent DNA polymerase: murine terminal deoxynucleotidyltransferase. EMBO J. 21 (3): 427-439.

Deshpande S, Yang Y, Chilkoti A, Zauscher S (2019). Enzymatic synthesis and modification of high molecular weight DNA using terminal deoxynucleotidyl transferase. Methods Enzymol. 627:163-188.

Dichl F, Li M, He Y, Kinzler K W, Vogelstein B, Dressman D (2006). BEAMing: single-molecule PCR on microparticles in water-in-oil emulsions. Nat Methods 3 (7): 551-559.

Domínguez O, Ruiz J F, Laín de Lera T, García-Díaz M, González M A, Kirchhoff T, Martínez-A C, Bernad A, Blanco L (2000). DNA polymerase mu (Pol mu), homologous to TdT, could act as a DNA mutator in eukaryotic cells. EMBO J. 19 (7): 1731-1742.

Efcavitch, W J, Sylvester J E (2016). Modified template-independent enzymes for deoxynucleotide synthesis. World Intellectual Property Organization patent application WO 2016/064880 A1.

Eisenbeis S, Höcker B (2010). Evolutionary mechanism as a template for protein engineering. J Pept Sci. 16 (10): 538-544.

Fiala K A, Brown J A, Ling H, Kshetry A K, Zhang J, Taylor J S, Yang W, Suo Z (2007). Mechanism of template-independent nucleotide incorporation catalyzed by a template-dependent DNA polymerase. J Mol Biol. 365 (3): 590-602.

Foo J L, Ching C B, Chang M W, Leong S S (2012). The imminent role of protein engineering in synthetic biology. Biotechnol Adv. 30 (3): 541-549.

Fowler J D, Suo Z (2006). Biochemical, structural, and physiological characterization of terminal deoxynucleotidyl transferase. Chem Rev. 106 (6): 2092-2110.

Frank E G, McLenigan M P, McDonald J P, Huston D, Mead S, Woodgate R (2017). DNA polymerase iota: The long and the short of it! DNA Repair (Amst). 58:47-51.

Ghadessy F J, Ong J L, Holliger P (2001). Directed evolution of polymerase function by compartmentalized self-replication. Proc Natl Acad Sci U S A 98 (8): 4552-4557.

Ghadessy F J, Holliger P (2007). Compartmentalized self-replication: a novel method for the directed evolution of polymerases and other enzymes. Methods Mol Biol. 352:237-248.

Global Oligonucleotide Synthesis Market Size, Industry Report, 2025. Grand View Research, San Francisco, CA, October 2018.

Golosov A A, Warren J J, Beese L S, Karplus M (2010). The mechanism of the translocation step in DNA replication by DNA polymerase I: a computer simulation analysis. Structure 18 (1): 83-93.

Gouge J, Rosario S, Romain F, Beguin P, Delarue M (2013). Structures of intermediates along the catalytic cycle of terminal deoxynucleotidyltransferase: dynamical aspects of the two-metal ion mechanism. J Mol Biol. 425 (22): 4334-4352.

Griffiths A D, Tawfik D S (2006). Miniaturising the laboratory in emulsion droplets. Trends Biotechnol. 24 (9): 395-402.

Guo C, Kosarck-Stancel J N, Tang T S, Friedberg E C (2009). Y-family DNA polymerases in mammalian cells. Cell Mol Life Sci. 66 (14): 2363-2381.

Hiatt A C, Rose F (1995). 3′ protected nucleotides for enzyme catalyzed template-independent creation of phosphodiester bonds. U.S. Pat. No. 5,763,594 and related patents.

Hiatt A C, Rose F (1995). Compositions for enzyme catalyzed template-independent creation of phosphodiester bonds using protected nucleotides. U.S. Pat. No. 5,808,045 and related patents.

Hoff K, Halpain M, Garbagnati G, Edwards JS, Zhou W (2020). Enzymatic Synthesis of Designer DNA Using Cyclic Reversible Termination and a Universal Template. ACS Synth Biol. 9 (2): 283-293.

Hogg M, Sauer-Eriksson A E, Johansson E (2012). Promiscuous DNA synthesis by human DNA polymerase teta. Nucleic Acids Res. 40 (6): 2611-22.

Hoitsma N M, Whitaker A M, Schaich M A, Smith M R, Fairlamb MS, Freudenthal B D (2020). Structure and function relationships in mammalian DNA polymerases. Cell Mol Life Sci. 77 (1): 35-59.

Jarosz D F, Beuning P J, Cohen S E, Walker G C (2007). Y-family DNA polymerases in Escherichia coli. Trends Microbiol. 15 (2): 70-77.

Jensen M A, Davis R W (2018). Template-Independent Enzymatic Oligonucleotide Synthesis (TiEOS): Its History, Prospects, and Challenges. Biochemistry 57 (12): 1821-1832.

Jensen M A, Griffin P, Davis R W (2018a). Free-running enzymatic oligonucleotide synthesis for data storage applications. bioRxiv June 2018. https://doi.org/10.1101/355719.

Johnson L B, Huber T R, Snow C D (2014). Methods for library-scale computational protein design. Methods Mol Biol. 1216:129-59.

Juárez R, Ruiz J F, Nick McElhinny S A, Ramsden D, Blanco L (2006). A specific loop in human DNA polymerase mu allows switching between creative and DNA-instructed synthesis. Nucleic Acids Res. 34 (16): 4572-4582.

Kaminski A M, Bebenek K, Pedersen L C, Kunkel T A (2020). DNA polymerase mu: An inflexible scaffold for substrate flexibility. DNA Repair (Amst). 93:102932.

Kaushik M, Sinha P, Jaiswal P, Mahendru S, Roy K, Kukreti S (2016). Protein engineering and de novo designing of a biocatalyst. J Mol Recognit. 29 (10): 499-503.

Kazlauskas D, Krupovic M, Guglielmini J, Forterre P, Venclovas Č (2020). Diversity and evolution of B-family DNA polymerases. Nucleic Acids Res. 48 (18): 10142-10156.

Kent T, Mateos-Gomez P A, Sfeir A, Pomerantz R T (2016). Polymerase teta is a robust terminal transferase that oscillates between three different mechanisms during end-joining. Elife 5:13740.

Leatherbarrow R J, Fersht A R (1986). Protein engineering. Protein Eng. 1 (1): 7-16.

Lee H, Wiegand D J, Griswold K, Punthambaker S, Chun H, Kohman R E, Church G M (2020). Photon-directed multiplexed enzymatic DNA synthesis for molecular digital data storage. Nat Commun. 11 (1): 5246.

Leisola M, Turunen O (2007). Protein engineering: opportunities and challenges. Appl Microbiol Biotechnol. 75 (6): 1225-1232.

Loc'h J, Delarue M (2018). Terminal deoxynucleotidyltransferase: the story of an untemplated DNA polymerase capable of DNA bridging and templated synthesis across strands. Curr Opin Struct Biol. 53:22-31.

Lutz S, Benkovic S J (2000). Homology-independent protein engineering. Curr Opin Biotechnol. 11 (4): 319-324.

Lutz S, Iamurri S M (2018). Protein Engineering: Past, Present, and Future. Methods Mol Biol. 1685:1-12.

Lcc H H, Kalhor R, Goela N, Bolot J, Church G M (2018). Enzymatic DNA synthesis for digital information storage. bioRxiv June 2018.

Lec H H, Kalhor R, Gocla N, Bolot J, Church G M (2019). Terminator-free template-independent enzymatic DNA synthesis for digital information storage. Nat Commun. 10 (1): 2383.

Marcheschi R J, Gronenberg L S, Liao J C (2013). Protein engineering for metabolic engineering: current and next-generation tools. Biotechnol J. 8 (5): 545-55.

Maxwell B A, Suo Z (2014). Recent insight into the kinetic mechanisms and conformational dynamics of Y-Family DNA polymerases. Biochemistry 3 (17): 2804-2814.

Miller O J, Bernath K, Agresti J J, Amitai G, Kelly B T, Mastrobattista E, Taly V, Magdassi S, Tawfik D S, Griffiths A D (2006). Directed evolution by in vitro compartmentalization. Nat Methods 3 (7): 561-570.

Moon, A F, Garcia-Diaz, M, Bebenck, K, Davis, B J, Zhong, X, Ramsden, D A, Kunkel T A, Pedersen, LC (2007). Structural insight into the substrate specificity of DNA Polymerase mu. Nat. Struct. Mol. Biol. 2007, 14 (1), 45-53.

Moon A F, Garcia-Diaz M, Batra VK, Beard W A, Bebenek K, Kunkel T A, Wilson S H, Pedersen L C (2007a). The X family portrait: structural insights into biological functions of X family polymerases. DNA Repair (Amst). 6 (12): 1709-1725.

Moon A F, Pryor J M, Ramsden D A, Kunkel T A, Bebenek K, Pedersen L C (2014). Sustained active site rigidity during synthesis by human DNA polymerase mu. Nat Struct Mol Biol. 21 (3): 253-260.

Motea E A, Berdis A J (2010). Terminal deoxynucleotidyl transferase: the story of a misguided DNA polymerase. Biochim Biophys Acta 1804 (5): 1151-1166.

Mueller R, Pajatsch M, Curdt I, Sobek H, Schmidt M, Suppmann B, Sonn K, Schneidinger B (2009). Recombinant terminal deoxynucleotidyl transferase with improved functionality. U.S. Pat. No. 7,494,797.

Oligonucleotide Synthesis Market. MarketsandMarkets™ Research Private Ltd., Pune, India, April 2019.

O'Fágáin C. Engineering protein stability (2011). Methods Mol Biol. 681:103-36.

Packer M S, Liu D R (2015). Methods for the directed evolution of proteins. Nat Rev Genet. 16 (7): 379-394.

Palluk S, Arlow D H, de Rond T, Barthel S, Kang JS, Bector R, Baghdassarian H M, Truong A N, Kim P W, Singh A K, Hillson N J, Keasling J D (2018). De novo DNA synthesis using polymerase-nucleotide conjugates. Nat Biotechnol. 36 (7): 645-650.

Perkel J M (2019). The race for enzymatic DNA synthesis heats up. Nature 566 (7745): 565.

Ramadan K, Shevelev I, Hübscher U (2004). The DNA-polymerase-X family: controllers of DNA quality? Nat Rev Mol Cell Biol. 5 (12): 1038-1043.

Rechkoblit O, Malinina L, Cheng Y, Kuryavyi V, Broyde S, Geacintov N E, Patel D J (2006). Stepwise translocation of Dpo4 polymerase during error-free bypass of an oxoG lesion. PLOS Biol. 4 (1): e11.

Ren Z (2016). Molecular events during translocation and proofreading extracted from 200 static structures of DNA polymerase. Nucleic Acids Res. 44 (15): 7457-7474.

Repasky J A, Corbett E, Boboila C, Schatz D G (2004). Mutational analysis of terminal deoxynucleotidyltransferase-mediated N-nucleotide addition in V (D) J recombination. J Immunol. 172 (9): 5478-5488.

Ruiz J F, Domínguez O, Laín de Lera T, Garcia-Díaz M, Bernad A, Blanco L (2001). DNA polymerase mu, a candidate hypermutase? Philos Trans R Soc Lond B Biol Sci. 356 (1405): 99-109.

Samkurashvili I, Luse D S (1996). Translocation and transcriptional arrest during transcript elongation by RNA polymerase II. J Biol Chem. 1996 Sep. 20;271 (38): 23495-23505.

Sarac I, Hollenstein M (2019). Terminal Deoxynucleotidyl Transferase in the Synthesis and Modification of Nucleic Acids. Chembiochem 20 (7): 860-871.

Schott H, Schrade H (1984). Single-step elongation of oligodeoxynucleotides using terminal deoxynucleotidyl transferase. Eur J Biochem. 143 (3): 613-620.

Shin H, Cho B K (2015). Rational Protein Engineering Guided by Deep Mutational Scanning. Int J Mol Sci. 16 (9): 23094-23110.

Singh R K, Lec J K, Selvaraj C, Singh R, Li J, Kim S Y, Kalia V C (2018). Protein Engineering Approaches in the Post-Genomic Era. Curr Protein Pept Sci. 19 (1): 5-15.

Sinha R, Shukla P (2019). Current Trends in Protein Engineering: Updates and Progress. Curr Protein Pept Sci. 20 (5): 398-407.

Swint-Kruse L (2016). Using Evolution to Guide Protein Engineering: The Devil IS in the Details. Biophys J. 111 (1): 10-18.

Takeuchi R, Choi M, Stoddard B L (2014). Redesign of extensive protein-DNA interfaces of meganucleases using iterative cycles of in vitro compartmentalization. Proc Natl Acad Sci USA. 111 (11): 4061-4066.

Tawfik D S, Griffiths A D (1998). Man-made cell-like compartments for molecular evolution. Nature Biotechnol. 16 (7): 652-656.

Tay Y, Ho C, Droge P, Ghadessy F J (2010). Selection of bacteriophage lambda integrases with altered recombination specificity by in vitro compartmentalization. Nucleic Acids Res. 38 (4): €25.

Trakselis M A, Murakami K S (2014). Introduction to Nucleic Acid Polymerases: Families, Themes, and Mechanisms. Nucl. Acids Mol. Biol. 30:1-15.

Uchiyama Y, Takeuchi R, Kodera H, Sakaguchi K (2009). Distribution and roles of X-family DNA polymerases in eukaryotes. Biochimie 91 (2): 165-170.

Vaisman A, Woodgate R (2017). Translesion DNA polymerases in eukaryotes: what makes them tick? Crit Rev Biochem Mol Biol. 2017 June;52 (3): 274-303.

Wilding M, Hong N, Spence M, Buckle A M, Jackson C J (2019). Protein engineering: the potential of remote mutations. Biochem Soc Trans. 47 (2): 701-711.

Woodley J M (2013). Protein engineering of enzymes for process applications. Curr Opin Chem Biol. 17 (2): 310-316.

Wrenbeck E E, Faber M S, Whitehead T A (2017). Deep sequencing methods for protein engineering and design. Curr Opin Struct Biol. 45:36-44.

Yamtich J, Sweasy J B (2010). DNA polymerase family X: function, structure, and cellular roles. Biochim Biophys Acta 1804 (5): 1136-1150.

Yang W (2014). An overview of Y-Family DNA polymerases and a case study of human DNA polymerase eta. Biochemistry 53 (17): 2793-2803.

Yang W, Gao Y (2018). Translesion and Repair DNA Polymerases: Diverse Structure and Mechanism. Annu Rev Biochem. 87:239-261.

Yang K K, Wu Z, Arnold F H (2019). Machine-learning-guided directed evolution for protein engineering. Nat Methods 16 (8): 687-694.

Zahn K E, Wallace S S, Doublié S (2011). DNA polymerases provide a canon of strategies for translesion synthesis past oxidatively generated lesions. Curr Opin Struct Biol. 21 (3): 358-369.

Zawaira A, Pooran A, Barichievy S, Chopera D (2012). A discussion of molecular biology methods for protein engineering. Mol Biotechnol. 51 (1): 67-102.

Zoller M J (1991). New molecular biology methods for protein engineering. Curr Opin Biotechnol. 2 (4): 526-531.

All publications, databases, GenBank sequences, patents and patent applications cited in this Specification are herein incorporated by reference as if each was specifically and individually indicated to be incorporated by reference.

1.-13. (canceled)

14. A process of synthesizing a desired nucleic acid comprising:
(a) Combining in a single vessel at least one nucleic acid substrate, an excess of free unblocked nucleoside triphosphate and at least one template independent nucleic acid polymerase having at least 85% identity to any one of SEQ ID NOs: 26, 6, 28, 8, 21-25, 27, 1-5 and 7;
(b) Reacting the mixture in part (a) under conditions in which the template independent nucleic acid polymerase is active and adds only a single nucleotide to each of the plurality of the nucleic acid substrate molecules present in the reaction to form a new nucleic acid molecule;
(c) Separating the new nucleic acid molecule from free nucleotides and the template independent nucleic acid polymerase; and
(d) Repeating steps (a)-(c) to obtain the desired synthesized nucleic acid, wherein the new nucleic acid molecule of step (c) serves as the at least one nucleic acid substrate of step (a) until the desired nucleic acid is synthesized.

15. The process according to claim 14, wherein the sequence identity of the template independent nucleic acid polymerase is at least 90%.

16. The process according to claim 15, wherein the sequence identity of the template independent nucleic acid polymerase is at least 95%.

17. The process according to claim 15, wherein 98%.

18. The process according to claim 15, wherein the sequence identity of the template independent nucleic acid polymerase is 100%.

19. A nucleic acid encoding a polypeptide at least 85% identical to SEQ ID NO: 8, SEQ ID NO: 28: SEO ID NO: 26, SEO ID NO: 6. SEO ID NO: 21-25, SEQ ID NO: 27, SEQ ID NO: 1.5, or SEQ ID NO: 7.

20. The nucleic acid according to claim 19, wherein the encoded polypeptide is SEQ ID NO: 28.

21. A polypeptide at least 85% identical to SEQ ID NO: 8, SEQ ID NO: 28, SEQ ID NO: 26, SEQ ID NO: 6, SEQ ID NO: 21-25, SEQ ID NO: 27, SEQ ID NO: 1.5, or SEQ ID NO: 7.

22. The polypeptide according to claim 21, where the polypeptide is SEQ ID NO: 28.

23. The nucleic acid according to claim 19, wherein the encoded polypeptide is SEQ ID NO: 6.

24. The polypeptide according to claim 21, wherein the polypeptide is SEQ ID NO: 6.

25. The nucleic acid according to claim 19, wherein the encoded polypeptide is SEQ ID NO: 26.

26. The polypeptide according to claim 21, wherein the polypeptide is SEQ ID NO: 26.

27. The nucleic acid according to claim 19, wherein the encoded polypeptide is SEQ ID NO: 8.

28. The polypeptide according to claim 21, wherein the polypeptide is SEQ ID NO: 8.

29. The process according to claim 14, wherein the at least one template independent nucleic acid polymerase is SEQ ID NO: 6 or 26.

30. The process according to claim 14, wherein the at least one template independent nucleic acid polymerase is SEQ ID NO: 8 or 28.

31. The process according to claim 14, wherein the at least one template independent nucleic acid polymerase is SEQ ID NO: 1 or 21.

32. The process according to claim 14, wherein the at least one template independent nucleic acid polymerase is SEQ ID NO: 2 or 22.

33. The process according to claim 14, wherein the at least one template independent nucleic acid polymerase is SEQ ID NO: 3 or 23.


SEQUENCE LISTING (54 sequences)

SEQ ID NO: 1 - protein, 632 aa, Saccharomyces cerevisiae
MSKFTWKELIQLGSPSKAYESSLACIAHIDMNAFFAQVEQMRCGLSKEDPVVCVQWNSIIAVSYAARKYGISRMDTIQEALKKCSNLIPIHTAVFKKGEDFWQYHDGCGSWVQDPAKQISVEDHKVSLEPYRRESRKALKIFKSACDLVERASIDEVFLDLGRICFNMLMFDNEYELTGDLKLKDALSNIREAFIGGNYDINSHLPLIPEKIKSLKFEGDVFNPEGRDLITDWDDVILALGSQVCKGIRDSIKDILGYTTSCGLSSTKNVCKLASNYKKPDAQTIVKNDCLLDFLDCGKFEITSFWTLGGVLGKELIDVLDLPHENSIKHIRETWPDNAGQLKEFLDAKVKQSDYDRSTSNIDPLKTADLAEKLFKLSRGRYGLPLSSRPVVKSMMSNKNLRGKSCNSIVDCISWLEVFCAELTSRIQDLEQEYNKIVIPRTVSISLKTKSYEVYRKSGPVAYKGINFQSHELLKVGIKFVTDLDIKGKNKSYYPLTKLSMTITNFDIIDLQKTVVDMFGNQVHTFKSSAGKEDEEKTTSSKADEKTPKLECCKYQVTFTDQKALQEHADYHLALKLSEGLNGAEESSKNLSFGEKRLLFSRKRPNSQHTATPQKKQVTSSKNILSFFTRKK

SEQ ID NO: 2 - protein, 498 aa, Takifugu rubripes
MFHATALPRMRKRPRPEEVACPGREDVKFRDVRLYLVEMKMGRSRRSFLTQLARSKGFMVEEVLSNRVTHVVSESSQAPVLWAWLKERAPQDLPNMHVVNITWFTDSMRESRPVAVETRHLIQDTLPAIPEGGAPAAEVSQYACQRRTTTDNYNVVFTDAFEVLAECYEFNQMDGRCLAFRRAASVLKSLPRGLSSLEETHSLPCLGGHAKAIIGEILQHGRAFDVEKVLSDERYQTLKLFTSVYGVGPKTAEKWYRSGLRSLDHILADQSIQLNHMQQNGFLHYGDISRAVSKAEARALTKAIGETVQAITPDALLALTGGFRRGKEFGHDVDIIFTTLELGMEENLLLAVIKSLEKQGILLYCDYQASTFDLTKLPTHSFEAMDHFAKCFLILRLEASQVEEGLNSPVEDIRGWRAVRVDLVSPPVDRYAFALLGWTGSRQFERDLRRFARKERRMLLDNHGLYDKTKEEFLAAGTEKDIFDHLGLEYMEPWQRNA

SEQ ID NO: 3 - protein, 568 aa, Candida glabrata
MGILSGKKFLILPNSHTGSVNILAGIVKEQGGFLVSSADRLSNDVVVLVNDSFVDKTNKIVNRGLFLKEFELDASVVWTYVLENELVCLRVSLVPSWVENGTFHFSDSERIILLDSESQERDTKNVQFHSAGNEEAGSDDETDVEGNKESTGDITDVSDTATPQLQSSPLSKYIKQEEDIDNQVLIKALGRLVKKYEVKGDQYRSRSYRLAKQAVEKYPHKITSGSQAQRQLSNIGSSIAKKIQLLLDTGTLPGLEDPATDEYESSLGYFSECYGIGVPMAKKWITLNISTFYRAARLHPKLFISDWPILYGWTYYEDWSKRIPRDEVTAHFELVKEEVRRVGNGCSVEMQGSYVRGARDTGDVDLMFYKENCDDLEEVTIGMENVAASLYQKGYIKCFLLLTDKLERMFRPDILSRLQKCGIAEISNEHTFRNSDRGKKLFFGVELPGDYPIYPFDDKDILQLKPQDKFMSKSKDAGHFCRRLDFFCCKWSELGAARIHYTGNTDYNRWLRVRAMDMGYKLTQHGIFKDDVLLESFDERKIFEYLHVPYLNPVDRNKTDWVNIPIPK

SEQ ID NO: 4 - protein, 530 aa, Wickerhamomyces ciferrii
MNRSGQVLSKMSKTYLFDGLEFLFIPNINSSKVTFTRKNLARNGGASVAKKFDQDTTTHVLVDTKVYLTKDKISAGLKNAKVPKTFQPGKILNQTWLVDSIEQQKLLDTKEYIIKLDELKPETRKESPASKQHIENLQKQETKEKLIAESSTGNPNERTIFLLNQMAEERLLQGEHFKAKAYKNAINALNNTGDFISDANEALRLKGIGVSVAQKIEEIVKTNTLSSLNEIKSDKEHQVSKLFMGIHGVGPVSAKKWYNDGLRTLEDVSQKPDLTSNQTLGLKYYDEWLERIPRDECTLHNEFMSDLVSQIDPLVQFTIGGSYRRGSPTCGDVDFIITKPNADNEEMKEILEKILVKIEQVGYLKCSLQKKHSTKFLSGCALPPNYASRLPEYSEGKWGKCRRIDFLMVPWKERGAAFIYFTGNDYFNRLIRLKAVKNGLVLNESGLFKRIKYVQGKNVEDKTMLIESFSEKKIFKLLGFKYVPPEQRNFGANNPPSKLGKHLDQFRIDHKYFDKVVKEEIIDDDVIEVD

SEQ ID NO: 5 - protein, 349 aa, Pseudomonas aeruginosa
MRKIIHIDCDCFYAALEMRDDPSLRGKALAVGGSPDKRGVVATCSYEARAYGVRSAMAMRTALKLCPDLLVVRPRFDVYRAVSKQIHAIFRDYTDLIEPLSLDEAYLDVSASPHFAGSATRIAQDIRRRVAEELRITVSAGVAPNKFLAKIASDWRKPDGLFVITPEQVDGFVAELPVAKLHGVGKVTAERLARMGIRTCADLRQGSKLSLVREFGSFGERLWGLAHGIDERPVEVDSRRQSVSVECTFDRDLPDLAACLEELPTLLEELDGRLQRLDGSYRPDKPFVKLKFHDFTQTTVEQSGAGRDLESYRQLLGQAFARGNRPVRLIGVGVRLLDLQGAHEQLRLF

SEQ ID NO: 6 - protein, 358 aa, Pigmentiphaga sp. H8
MRKIIHCDCDCFYASIEMRDDPSLRGRPLAVGGRPETRGVVATCNYEARKYGVHSAMSSARAVRLCPDLLIIPPRMEMYRVASAQIMDIYRDYTELVEPLSLDEAYLDVTGSDRLQGSATRIASEIRQRVAQAVGITVSAGVAPSKFVAKIASDWNKPDGLFVVRPQDVDTFVAALPVAKLHGVGKVTGARLKALGVETCADLREWEHDRLRDEFGAFGERLHDLCRGIDLREVSPTRERKSVSVEQTFVTDLHTLEACQALLREMLDQLDARVRRADAQNHIQKLFVKLRFSDFNRTTAEGVGAALDEEQFRILLATAFRRNPRAVRLMGLGVRLGAPGGQLALFGDQPTVSEPDTV

SEQ ID NO: 7 - protein, 502 aa, Xenopus tropicalis
MSFIPLKRRRAGPVSEEPLDSLQSLFPDVCLFLVERRMGSARRKFLTGLAQKKGFCVTPQFSDQVTHVVSEQNSCSEVLLWIERQSGQKVQPGGAEMTPHILDITWFTESMSLGKPVKVEPRHCLGVSDSSVSRDKATQEIPAYGCQRRTPLHHHNKEITDALEILALSASFQGSEARFLGFTRASSVLKSLPFRLQSVEEVKDLPWCGGHSQTVIQEILEDGVCREVETVKNSEHFQSMKALTSIFGVGIRTADKWYRDGVRSLSDLNNLGGKLTAEQKAGLLHYTDLQQSVTREEAGTVEQLIKGALQSFVPDVRVTMTGGFRRGKQEGHDVDFLITHPDEEALNGLLRKAVAWLDGKGSVLYYHVRARSQNFSGSNTMDGHETCYSIIALPNVCPEKPSPDAEKIEPDLDKNSLRNWKAVRVDLVVCPYSEYFYALLGWTGSKHFERELRRFSLHVKKMSLNSHGLFDIQKKCHHPATSEEEIFAHLGLPYVPPSERNA

SEQ ID NO: 8 - protein, 530 aa, Wickerhamomyces ciferrii; identical to SEQ ID NO: 4

SEQ ID NO: 9 - protein, 520 aa, Bos taurus
MAQQRQHQRLPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 10 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 11 - DNA, 1923 bp, Artificial Sequence
  Cloned EDS017 sequence with His6 tag
atgcatcatcatcaccatcacggcagcagcaagtttacctggaaagaactgattcagctgggtagcccgagcaaagcatatgaaagcagcctggcatgtattgcccatattgatatgaatgcatttttcgcacaggttgagcagatgcgttgtggtctgagcaaagaagatccggttgtttgcgttcagtggaatagcattattgcagttagctatgcagcccgtaaatatggtattagccgtatggataccattcaagaggcactgaaaaaatgcagcaatctgattccgattcataccgcagttttcaaaaaaggcgaagatttttggcagtatcatgatggttgtggtagctgggttcaagatccggcaaaacaaatttcagtcgaagatcataaagttagcctggaaccgtatcgtcgtgaaagccgtaaagccctgaaaatctttaaaagcgcatgtgatctggttgaacgtgcaagcattgatgaagtttttctggatctgggtcgcatttgttttaacatgctgatgttcgataacgagtatgaactgaccggtgatctgaaactgaaagatgcactgagcaatattcgcgaagcatttattggtggcaactatgatattaacagccatctgccgctgattccggaaaaaatcaaaagcctgaaattcgaaggcgacgtgtttaatccggaaggtcgtgatctgattacagattgggatgatgttattctggcactgggtagtcaggtttgtaaaggtattcgtgatagcatcaaagatatcctgggttataccacctcatgtggtctgtcaagcaccaaaaatgtttgtaaactggccagcaactacaaaaaaccggatgcacagaccattgtgaaaaatgattgtctgctggatttcctggattgcggcaaatttgaaattaccagcttttggaccttaggtggtgttctgggtaaagaattaattgatgtgctggatctgccgcatgaaaacagcattaaacatattcgtgaaacctggcctgataatgcaggtcagctgaaagaatttctggatgccaaagttaaacagagcgattatgatcgtagcaccagcaatattgatccgctgaaaaccgcagatctggccgaaaaactgtttaaactgagccgtggtcgttatggcctgccgctgtcaagccgtccggttgtgaaaagcatgatgagcaataaaaacctgcgtggcaaaagctgcaatagcattgttgattgtattagctggctggaagttttttgtgcagaactgaccagccgtattcaggatctggaacaagaatataacaagatcgttattccgcgtaccgttagcattagcctgaaaaccaaaagctatgaggtgtatcgtaaaagcggtccggtggcatataaaggtatcaattttcagagccacgaactgctgaaagtgggtatcaaatttgtgaccgatctggatatcaaaggcaagaacaaaagttattacccgctgaccaaactgagcatgaccattaccaatttcgatatcatcgatctgcagaaaaccgtggttgatatgtttggtaatcaggtgcatacgtttaaaagcagcgcaggtaaagaagatgaagaaaaaaccaccagtagcaaagccgatgaaaaaaccccgaaactggaatgttgtaaatatcaggttaccttcaccgatcagaaagcactgcaagaacatgcagattatcatctggccctgaaactgtctgaaggtctgaatggtgcagaagaaagcagcaaaaatctgagctttggtgaaaaacgtctgctgtttagccgtaaacgtccgaatagccagcataccgcaacaccgcagaaaaaacaggttaccagcagtaaaaacatcctgagcttttttacccgcaaaaaatga

SEQ ID NO: 12 - DNA, 1521 bp, Artificial Sequence
  Cloned EDS024 sequence with His6 tag
atgcatcatcatcaccatcacggcagctttcatgcaaccgcactgcctcgtatgcgtaaacgtccgcgtccggaagaagttgcctgtccgggtcgtgaagatgttaaatttcgtgatgttcgtctgtacctggtggaaatgaaaatgggtcgtagccgtcgtagctttctgacccagctggcacgtagcaaaggttttatggttgaagaggttctgagcaatcgtgttacccatgttgttagcgaaagcagccaggcaccggttctgtgggcatggctgaaagaacgtgcaccgcaggatctgccgaatatgcatgttgtgaatattacctggtttaccgatagcatgcgtgaaagccgtccggttgcagttgaaacccgtcatctgattcaggataccctgcctgcaattccggaaggtggtgcaccggcagccgaagttagccagtatgcatgtcagcgtcgtaccaccaccgataactataatgttgtttttaccgatgcctttgaagttctggccgaatgctatgaatttaatcagatggatggtcgttgtctggcatttcgtcgtgcagcaagcgttctgaaaagcctgcctcgtggtctgagcagcctggaagaaacccatagcctgccgtgtttaggtggtcatgcaaaagcaattattggcgaaattctgcagcatggtcgtgcatttgatgttgaaaaagttctgagtgatgaacgctatcagaccctgaaactgtttaccagcgtttatggtgttggtccgaaaaccgcagaaaaatggtatcgtagcggtctgcgtagcctggatcatattctggcggatcagagcatccagctgaatcatatgcagcagaatggttttctgcattatggtgatattagccgtgcagttagcaaagccgaagcacgtgcactgaccaaagcaattggtgaaaccgttcaggcaattacaccggatgcactgctggcactgaccggtggttttcgtcgcggtaaagaatttggtcatgatgtggatattatctttaccacgctggaattaggcatggaagaaaatctgctgctggcagtgattaaaagtctggaaaaacagggtattctgctgtattgtgattatcaggcaagcacctttgatctgaccaaactgccgacacatagctttgaagcaatggatcattttgccaagtgctttctgattctgcgtctggaagcaagccaggttgaagaaggcctgaatagtccggttgaagatattcgtggttggcgtgcagttcgtgttgatctggttagccctccggttgatcgttatgcatttgcactgttaggttggaccggtagccgtcagtttgaacgtgatctgcgtcgttttgcacgtaaagaacgtcgtatgctgctggataatcatggcctgtatgataaaaccaaagaagaatttctggcagccggtacggaaaaagatatttttgatcatctgggccttgagtatatggaaccgtggcagcgtaatgcataa

SEQ ID NO: 13 - DNA, 1731 bp, Artificial Sequence
  Cloned EDS029 sequence with His6 tag
atgcatcatcatcaccatcacggcagcggtattctgagcggcaaaaaattcctgattctgccgaatagccataccggtagcgttaatattctggcaggtattgttaaagaacaaggtggttttctggttagcagcgcagatcgtctgagcaatgatgttgttgttctggtgaatgatagcttcgtggacaaaaccaacaaaattgttaatcgcggtctgtttctgaaagaatttgaactggatgcaagcgttgtttggacctatgttctggaaaatgaactggtttgtctgcgtgttagcctggttccgagctgggttgaaaatggcacctttcattttagcgatagcgaacgtattattctgctggatagcgaaagccaagaacgcgataccaaaaatgttcagtttcatagcgcaggtaatgaagaggcaggtagtgatgatgaaaccgatgttgaaggtaataaagaaagcaccggtgatattaccgatgttagcgataccgcaacaccgcagctgcagagcagtccgctgagcaaatatatcaaacaagaagaggatatcgacaaccaggttctgattaaagcactgggtcgtctggtgaaaaaatacgaagttaaaggtgatcagtatcgcagccgtagctatcgtctggcaaaacaggcagttgaaaaatatccgcataaaatcaccagcggtagccaggcacagcgtcagctgagcaatattggtagcagcattgccaaaaaaatccagctgctgctggacaccggtacactgcctggtctggaagatccggcaaccgatgaatatgaaagcagcctgggttatttcagcgaatgttatggtattggtgttccgatggccaaaaaatggattaccctgaatatcagcaccttttatcgtgcagcacgtctgcatccgaaactgtttattagcgattggccgattctgtatggctggacctattatgaagattggagcaaacgtattccgcgtgatgaagttaccgcacattttgagctggttaaagaagaagttcgtcgcgttggtaatggttgtagcgttgaaatgcagggtagctatgttcgtggtgcacgtgataccggtgatgttgatctgatgttctacaaagaaaattgcgacgatctggaagaggttaccattggtatggaaaatgttgcagcaagcctgtatcagaaaggctatatcaaatgttttctgctgctgaccgataaactggaacgcatgtttcgtccggatattctgagtcgtctgcagaaatgtggtattgccgaaatcagcaatgaacatacctttcgtaatagcgaccgtggcaaaaaactgtttttcggtgttgaactgccaggcgattatccgatttatccgtttgatgataaagacatcctgcagctgaaaccgcaggataaattcatgagcaaaagcaaagatgccggtcatttttgtcgtcgtctggatttcttctgttgcaaatggtcagaactgggtgcagcccgtattcattataccggtaataccgattataaccgttggctgcgtgttcgtgcaatggatatgggttataaactgacccagcatggcatcttcaaagatgatgtactgctggaaagctttgatgagcgcaaaatctttgaatatctgcatgtgccgtatctgaatccggttgatcgtaataaaaccgattgggtgaatatcccgattccgaaataa

SEQ ID NO: 14 - DNA, 1617 bp, Artificial Sequence
  Cloned EDS030 sequence with His6 tag
atgcatcatcatcaccatcacggcagcaatcgtagcggtcaggttctgagcaaaatgagtaaaacctacctgtttgatggcctggaatttctgtttattccgaacattaatagcagcaaggtgacctttacacgcaaaaatctggcacgtaatggtggtgcaagcgttgccaaaaaattcgatcaggataccaccacacatgttctggttgataccaaagtttatctgaccaaagacaaaattagcgcaggtctgaaaaatgccaaagtgccgaaaacctttcagcctggtaaaattctgaatcagacctggctggttgattctattgaacagcagaaactgctggacaccaaagagtatattatcaaactggatgagctgaaaccggaaacgcgtaaagaaagtccggcaagcaaacagcatattgaaaatctgcagaaacaagaaaccaaagagaaactgattgcagaaagcagcaccggtaatccgaatgaacgtaccatttttctgctgaaccagatggcagaagaacgtctgctgcagggtgaacattttaaagcaaaagcctataagaacgccattaacgccctgaataataccggtgattttatctcagatgcaaatgaagcactgcgcctgaaaggtattggtgttagcgtggcacagaaaattgaagaaattgtgaaaaccaatacgctgagcagcctgaatgaaatcaaaagcgataaagaacaccaggtgagcaaactgtttatgggtattcatggtgttggtccggttagcgcaaaaaagtggtataatgatggtctgcgtaccctggaagatgttagccagaaaccggatctgaccagcaatcagaccctgggcctgaaatattacgatgaatggctggaacgtattccgcgtgatgaatgtaccctgcataatgaatttatgagcgatctggtgagccagattgatccgctggttcagtttaccattggtggtagctatcgtcgtggtagcccgacctgtggtgatgtggattttatcattaccaaaccgaatgccgataacgaagagatgaaagagattctggaaaagatcctggtgaaaatcgaacaggttggttatctgaaatgtagcctgcagaaaaaacacagcaccaaatttctgagcggttgtgcactgcctccgaattatgcaagccgtctgccggaatacagcgaaggtaaatggggtaaatgtcgtcgtattgattttctgatggttccgtggaaagaacgtggtgcagcatttatctattttaccggcaacgattatttcaaccgtctgattcgtctgaaagccgttaaaaatggtctggtgctgaatgaatcaggtctgtttaaacgcatcaaatacgtgcagggtaaaaacgtggaagataaaaccatgctgatcgaaagctttagcgagaaaaaaatctttaagctgctgggcttcaaatatgttccgcctgaacagcgtaattttggtgcaaataatccgcctagcaaactgggtaaacatctggatcagtttcgcatcgatcacaaatatttcgacaaagtggtgaaagaagagatcattgacgacgatgttatcgaggtggattaa

SEQ ID NO: 15 - DNA, 1074 bp, Artificial Sequence
  Cloned EDS053 sequence with His6 tag
atgcatcatcatcaccatcacggcagccgcaaaatcatccatattgattgcgattgcttttacgcagcactggaaatgcgtgatgatccgagcctgcgtggtaaagcactggcagttggtggtagtccggataaacgtggtgttgttgcaacctgtagctatgaagcacgtgcatatggtgttcgtagcgcaatggcaatgcgtaccgcactgaaactgtgtccggatctgctggttgttcgtccgcgttttgatgtttatcgtgcagttagcaaacaaatccatgccatctttcgtgattataccgatctgattgaaccgctgagcctggatgaagcatatctggatgttagcgcaagtccgcattttgcaggtagcgcaacccgtattgcacaggatattcgtcgtcgtgttgcagaagaactgcgtattaccgttagtgccggtgttgcaccgaacaaatttctggcaaaaattgcaagcgattggcgtaaaccggatggtctgtttgttattacaccggaacaggttgatggttttgttgccgaactgccggttgcaaaactgcatggtgttggtaaagttaccgcagaacgtctggcacgtatgggtattcgtacctgtgccgatctgcgtcagggtagcaaactgagtctggttcgtgaatttggtagctttggtgaacgtctgtggggtttagcacatggtattgatgaacgtccggttgaagttgatagccgtcgtcagagcgttagcgttgaatgtacctttgatcgtgatctgccggatctggcagcatgtctggaagaattaccgacactgctggaagaactggatggtcgtctgcagcgtctggatggtagctatcgtcctgataaaccgtttgtgaaactgaaattccacgattttacccagaccaccgttgaacagagcggtgcaggtcgcgatctggaaagttatcgtcagctgctgggtcaagcatttgcacgtggtaatcgtccggttcgtctgattggtgtgggtgttcgtctgctggatctgcagggtgcacatgaacagctgcgtctgttttaa

SEQ ID NO: 16 - DNA, 1101 bp, Artificial Sequence
  Cloned EDS054 sequence with His6 tag
atgcatcatcatcaccatcacggcagccgcaaaatcattcattgtgattgcgattgcttttacgccagcattgaaatgcgtgatgatccgagcctgcgtggtcgtccgctggcagttggtggccgtccggaaacacgtggtgttgttgcaacctgtaattatgaagcacgtaaatatggtgttcatagcgcaatgagcagcgcacgtgcagttcgtctgtgtccggatctgctgattattccgcctcgtatggaaatgtatcgtgttgcaagcgcacagatcatggatatttatcgtgattataccgaactggttgaaccgctgagcctggatgaagcatatctggatgttaccggtagcgatcgtctgcagggtagcgcaacccgtattgcaagcgaaattcgtcagcgtgttgcacaggccgttggtattaccgttagtgccggtgttgcaccgagcaaatttgttgccaaaattgccagcgattggaataaaccggatggtctgtttgttgttcgtccgcaggatgttgatacctttgttgcagcactgccggttgcaaaactgcatggtgttggtaaagttaccggtgcacgtctgaaagcactgggtgttgaaacctgtgccgatctgcgtgaatgggaacatgatcgtttacgtgatgaatttggtgcatttggtgaacgtctgcacgatctgtgtcgtggtattgatctgcgcgaagttagcccgacacgtgaacgtaaaagcgttagcgttgaacagacctttgttaccgatctgcataccctggaagcatgtcaggcactgctgcgtgaaatgctggatcagctggatgcacgtgttcgtcgtgcagatgcacagaaccatattcagaaactgtttgtgaaactgcgcttcagcgattttaatcgtaccacagccgaaggtgttggtgccgcactggatgaggaacagtttcgtattctgctggcaaccgcatttcgtcgtaatccgcgtgccgtgcgtctgatgggtctgggtgttcgtctgggtgcacctggtggtcagctggcactgtttggtgatcagccgaccgttagcgaaccggataccgtttaa

SEQ ID NO: 17 - DNA, 1533 bp, Artificial Sequence
  Cloned EDS066 sequence with His6 tag
atgcatcatcatcaccatcacggcagcagctttattccgctgaaacgtcgtcgtgcaggtccggttagcgaagaaccgctggatagcctgcagagcctgtttccggatgtttgtctgtttctggttgaacgtcgtatgggtagcgcacgtcgtaaatttctgaccggtctggcacagaaaaaaggtttttgtgttacaccgcagtttagcgatcaggttacccatgttgttagcgaacagaatagctgtagcgaagttctgctgtggattgaacgtcagagtggtcagaaagttcagcctggtggtgcagaaatgacaccgcatattctggatattacctggtttaccgaaagcatgagcctgggtaaaccggttaaagttgaaccgcgtcattgtctgggtgttagcgatagcagcgttagccgtgataaagcaacccaagaaattccggcatatggttgtcagcgtcgtacaccgctgcatcatcataataaagaaattaccgatgcgctggaaattctggcactgagcgcaagctttcagggtagcgaagcacgttttctgggttttacccgtgcaagcagcgttctgaaaagcctgccgtttcgtctgcagagcgttgaagaggttaaagatctgccgtggtgtggtggtcatagccagaccgttattcaagaaatcctggaagatggtgtttgccgtgaagttgaaaccgtgaaaaatagcgaacatttccagagcatgaaagcactgaccagcatttttggtgttggtattcgtaccgcagataaatggtatcgtgatggtgttcgtagcctgagcgatctgaataatcttggtggtaaactgaccgcagaacagaaagcaggtctgctgcattacaccgatctgcagcagagcgtgacccgtgaagaagcaggcaccgttgaacagctgattaaaggtgcactgcagagctttgtgccggatgtgcgtgttaccatgaccggtggttttcgtcgtggtaaacaagagggtcatgatgtggattttctgattacccatcctgatgaagaagccctgaacggcctgctgcgtaaagcagttgcatggctggatggtaaaggtagcgttctgtattatcatgttcgtgcacgtagtcagaattttagcggtagcaataccatggatggtcatgaaacctgttatagcattattgcactgccgaatgtttgtccggaaaaaccgagtccggatgcagaaaaaattgaaccggatctggataaaaacagcctgcgtaattggaaagcagttcgtgttgatctggttgtttgcccgtatagcgaatacttttatgcactgttaggttggaccggcagcaaacattttgaacgtgaactgcgtcgttttagcctgcatgtgaaaaaaatgagcctgaatagccatggcctgtttgacattcagaaaaagtgtcatcatccggcaaccagcgaagaagaaatttttgcacatctgggtctgccgtatgttccgcctagcgaacgtaatgcataa

SEQ ID NO: 18 - DNA, 1317 bp, Artificial Sequence
  Cloned EDS082 sequence with His6 tag
atgcatcatcatcaccatcacggcagcgaacagcagaaactgctggacaccaaagagtatattatcaaactggatgagctgaaaccggaaacgcgtaaagaaagtccggcaagcaaacagcatattgaaaatctgcagaaacaagaaaccaaagagaaactgattgcagaaagcagcaccggtaatccgaatgaacgtaccatttttctgctgaaccagatggcagaagaacgtctgctgcagggtgaacattttaaagcaaaagcctataagaacgccattaacgccctgaataataccggtgattttatctcagatgcaaatgaagcactgcgcctgaaaggtattggtgttagcgtggcacagaaaattgaagaaattgtgaaaaccaatacgctgagcagcctgaatgaaatcaaaagcgataaagaacaccaggtgagcaaactgtttatgggtattcatggtgttggtccggttagcgcaaaaaagtggtataatgatggtctgcgtaccctggaagatgttagccagaaaccggatctgaccagcaatcagaccctgggcctgaaatattacgatgaatggctggaacgtattccgcgtgatgaatgtaccctgcataatgaatttatgagcgatctggtgagccagattgatccgctggttcagtttaccattggtggtagctatcgtcgtggtagcccgacctgtggtgatgtggattttatcattaccaaaccgaatgccgataacgaagagatgaaagagattctggaaaagatcctggtgaaaatcgaacaggttggttatctgaaatgtagcctgcagaaaaaacacagcaccaaatttctgagcggttgtgcactgcctccgaattatgcaagccgtctgccggaatacagcgaaggtaaatggggtaaatgtcgtcgtattgattttctgatggttccgtggaaagaacgtggtgcagcatttatctattttaccggcaacgattatttcaaccgtctgattcgtctgaaagccgttaaaaatggtctggtgctgaatgaatcaggtctgtttaaacgcatcaaatacgtgcagggtaaaaacgtggaagataaaaccatgctgatcgaaagctttagcgagaaaaaaatctttaagctgctgggcttcaaatatgttccgcctgaacagcgtaattttggtgcaaataatccgcctagcaaactgggtaaacatctggatcagtttcgcatcgatcacaaatatttcgacaaagtggtgaaagaagagatcattgacgacgatgttatcgaggtggattaa

SEQ ID NO: 19 - DNA, 1176 bp, Artificial Sequence
  Cloned EDS048 sequence with His6 tag
atgcatcatcatcaccatcacggcagccgtaccgattatagcgcaaccccgaatccgggttttcagaaaacaccgcctctggcagtgaaaaaaatcagccagtatgcatgtcagcgtaaaaccacactgaataactataaccacatcttcaccgatgcctttgaaattctggcagaaaacagcgaattcaaagaaaacgaagttagctacgtgacctttatgcgtgcagcaagcgttctgaaaagcctgccgtttaccattattagcatgaaagataccgaaggtattccgtgtctgggtgataaagtgaaatgcatcattgaagagatcatcgaagatggtgaaagcagcgaagttaaagcagttctgaatgatgaacgttaccagagcttcaaactgtttaccagcgtttttggtgttggcctgaaaaccagcgaaaaatggtttcgtatgggttttcgtagcctgagcaaaatcatgagcgataaaaccctgaaattcaccaaaatgcagaaagccggtttcctgtattatgaagatctggtgagctgtgttacccgtgccgaagccgaagcagttggtgttctggttaaagaagcagtttgggcatttctgccggatgcatttgttaccatgaccggtggttttcgtcgtggcaaaaaaatcggtcatgatgtggattttctgattaccagtccgggtagcgcagaagatgaagaacagctgctgccgaaagttattaatctgtgggaaaaaaaaggcctgctgctgtattacgatctggttgaaagcaccttcgagaaattcaaactgccgagccgtcaggttgataccctggatcactttcagaaatgttttcttatcctgaagctgcatcatcagcgtgttgatagcagcaaaagcaatcagcaagaaggtaaaacctggaaagcaattcgtgttgatctggttatgtgcccgtatgaaaatcgtgcatttgcactgttaggttggaccggtagtcgtcagtttgaacgtgatattcgtcgttatgcaacccatgaacgtaaaatgatgctggataatcatgccctgtacgataaaacgaaacgcgtgttcctgaaagccgaaagcgaagaagaaatttttgcacatctgggccttgattacattgaaccgtgggaacgtaatgcctaa

SEQ ID NO: 20 - DNA, 1554 bp, Artificial Sequence
  Cloned EDS015 sequence with His6 tag
atgcatcatcatcaccatcacggcagcgatccgctgcaggcagttcatctgggtccgcgtaaaaaacgtccgcgtcagctgggtacaccggttgcaagcaccccgtatgatattcgttttcgtgatctggttctgttcatcctggaaaaaaagatgggtacaacccgtcgtgcatttctgatggaactggcacgtcgtaaaggttttcgtgttgaaaatgaactgagcgatagcgttacccatattgttgcagaaaataacagcggtagtgatgttctggaatggctgcaactgcagaacattaaagcaagcagcgaactggaactgctggatattagctggctgattgaatgtatgggtgcaggtaaaccggttgaaatgatgggtcgtcatcagctggttgttaatcgtaatagcagcccgagtccggttccgggtagccagaatgttccggcaccggcagtgaaaaaaatcagtcagtatgcatgtcagcgtcgtaccacactgaataactataatcagctgtttaccgatgcactggatattctggcagaaaatgatgagctgcgcgaaaatgaaggtagctgtctggcatttatgcgtgccagcagcgttctgaaaagcctgccgtttccgattaccagcatgaaagataccgaaggtattccgtgtctgggtgataaagtgaaaagcattattgaaggcatcatcgaagatggcgaaagcagtgaagcaaaagcagttctgaatgatgaacgctacaaaagcttcaaactgtttaccagcgtttttggtgttggtctgaaaaccgcagaaaaatggtttcgtatgggttttcgtaccctgagcaaaattcagagcgataaaagtctgcgttttacccagatgcagaaagcaggttttctgtattatgaagatctggtgagctgcgttaatcgtccggaagccgaagcagttagcatgctggttaaagaagcagttgttacctttctgccggatgcgctggttaccatgaccggtggttttcgtcgcggaaaaatgacaggtcatgatgtggattttctgattacctcaccggaagcaaccgaagatgaagaacagcaactgctgcataaagttaccgatttttggaaacagcagggtctgctgctgtattgtgatatcctggaatcaaccttcgagaaattcaaacagccgagccgtaaagttgatgccctggatcattttcagaagtgttttctgatcctgaaactggatcatggtcgtgttcatagcgaaaaaagcggtcagcaagaaggtaaaggttggaaagcaattcgtgtggatctggttatgtgtccgtatgatcgtcgtgcctttgcactgttaggttggaccggtagccgtcagtttgaacgtgatctgcgtcgttatgcaacccatgaacgtaaaatgatgctggataatcatgcactgtatgatcgcaccaaacgtgtttttctggaagcagaaagcgaagaagaaatctttgcacatctgggccttgattacattgaaccgtgggaacgtaatgca

SEQ ID NO: 21 - protein, 640 aa, Artificial Sequence
  EDS017 expressed protein sequence with His6 tag
MHHHHHHGSSKFTWKELIQLGSPSKAYESSLACIAHIDMNAFFAQVEQMRCGLSKEDPVVCVQWNSIIAVSYAARKYGISRMDTIQEALKKCSNLIPIHTAVFKKGEDFWQYHDGCGSWVQDPAKQISVEDHKVSLEPYRRESRKALKIFKSACDLVERASIDEVFLDLGRICFNMLMFDNEYELTGDLKLKDALSNIREAFIGGNYDINSHLPLIPEKIKSLKFEGDVFNPEGRDLITDWDDVILALGSQVCKGIRDSIKDILGYTTSCGLSSTKNVCKLASNYKKPDAQTIVKNDCLLDFLDCGKFEITSFWTLGGVLGKELIDVLDLPHENSIKHIRETWPDNAGQLKEFLDAKVKQSDYDRSTSNIDPLKTADLAEKLFKLSRGRYGLPLSSRPVVKSMMSNKNLRGKSCNSIVDCISWLEVFCAELTSRIQDLEQEYNKIVIPRTVSISLKTKSYEVYRKSGPVAYKGINFQSHELLKVGIKFVTDLDIKGKNKSYYPLTKLSMTITNFDIIDLQKTVVDMFGNQVHTFKSSAGKEDEEKTTSSKADEKTPKLECCKYQVTFTDQKALQEHADYHLALKLSEGLNGAEESSKNLSFGEKRLLFSRKRPNSQHTATPQKKQVTSSKNILSFFTRKK

SEQ ID NO: 22 - protein, 506 aa, Artificial Sequence
  EDS024 expressed protein sequence with His6 tag
MHHHHHHGSFHATALPRMRKRPRPEEVACPGREDVKFRDVRLYLVEMKMGRSRRSFLTQLARSKGFMVEEVLSNRVTHVVSESSQAPVLWAWLKERAPQDLPNMHVVNITWFTDSMRESRPVAVETRHLIQDTLPAIPEGGAPAAEVSQYACQRRTTTDNYNVVFTDAFEVLAECYEFNQMDGRCLAFRRAASVLKSLPRGLSSLEETHSLPCLGGHAKAIIGEILQHGRAFDVEKVLSDERYQTLKLFTSVYGVGPKTAEKWYRSGLRSLDHILADQSIQLNHMQQNGFLHYGDISRAVSKAEARALTKAIGETVQAITPDALLALTGGFRRGKEFGHDVDIIFTTLELGMEENLLLAVIKSLEKQGILLYCDYQASTFDLTKLPTHSFEAMDHFAKCFLILRLEASQVEEGLNSPVEDIRGWRAVRVDLVSPPVDRYAFALLGWTGSRQFERDLRRFARKERRMLLDNHGLYDKTKEEFLAAGTEKDIFDHLGLEYMEPWQRNA

SEQ ID NO: 23 - protein, 576 aa, Artificial Sequence
  EDS029 expressed protein sequence with His6 tag
MHHHHHHGSGILSGKKFLILPNSHTGSVNILAGIVKEQGGFLVSSADRLSNDVVVLVNDSFVDKTNKIVNRGLFLKEFELDASVVWTYVLENELVCLRVSLVPSWVENGTFHFSDSERIILLDSESQERDTKNVQFHSAGNEEAGSDDETDVEGNKESTGDITDVSDTATPQLQSSPLSKYIKQEEDIDNQVLIKALGRLVKKYEVKGDQYRSRSYRLAKQAVEKYPHKITSGSQAQRQLSNIGSSIAKKIQLLLDTGTLPGLEDPATDEYESSLGYFSECYGIGVPMAKKWITLNISTFYRAARLHPKLFISDWPILYGWTYYEDWSKRIPRDEVTAHFELVKEEVRRVGNGCSVEMQGSYVRGARDTGDVDLMFYKENCDDLEEVTIGMENVAASLYQKGYIKCFLLLTDKLERMFRPDILSRLQKCGIAEISNEHTFRNSDRGKKLFFGVELPGDYPIYPFDDKDILQLKPQDKFMSKSKDAGHFCRRLDFFCCKWSELGAARIHYTGNTDYNRWLRVRAMDMGYKLTQHGIFKDDVLLESFDERKIFEYLHVPYLNPVDRNKTDWVNIPIPK

SEQ ID NO: 24 - protein, 538 aa, Artificial Sequence
  EDS030 expressed protein sequence with His6 tag
MHHHHHHGSNRSGQVLSKMSKTYLFDGLEFLFIPNINSSKVTFTRKNLARNGGASVAKKFDQDTTTHVLVDTKVYLTKDKISAGLKNAKVPKTFQPGKILNQTWLVDSIEQQKLLDTKEYIIKLDELKPETRKESPASKQHIENLQKQETKEKLIAESSTGNPNERTIFLLNQMAEERLLQGEHFKAKAYKNAINALNNTGDFISDANEALRLKGIGVSVAQKIEEIVKTNTLSSLNEIKSDKEHQVSKLFMGIHGVGPVSAKKWYNDGLRTLEDVSQKPDLTSNQTLGLKYYDEWLERIPRDECTLHNEFMSDLVSQIDPLVQFTIGGSYRRGSPTCGDVDFIITKPNADNEEMKEILEKILVKIEQVGYLKCSLQKKHSTKFLSGCALPPNYASRLPEYSEGKWGKCRRIDFLMVPWKERGAAFIYFTGNDYFNRLIRLKAVKNGLVLNESGLFKRIKYVQGKNVEDKTMLIESFSEKKIFKLLGFKYVPPEQRNFGANNPPSKLGKHLDQFRIDHKYFDKVVKEEIIDDDVIEVD

SEQ ID NO: 25 - protein, 357 aa, Artificial Sequence
  EDS053 expressed protein sequence with His6 tag
MHHHHHHGSRKIIHIDCDCFYAALEMRDDPSLRGKALAVGGSPDKRGVVATCSYEARAYGVRSAMAMRTALKLCPDLLVVRPRFDVYRAVSKQIHAIFRDYTDLIEPLSLDEAYLDVSASPHFAGSATRIAQDIRRRVAEELRITVSAGVAPNKFLAKIASDWRKPDGLFVITPEQVDGFVAELPVAKLHGVGKVTAERLARMGIRTCADLRQGSKLSLVREFGSFGERLWGLAHGIDERPVEVDSRRQSVSVECTFDRDLPDLAACLEELPTLLEELDGRLQRLDGSYRPDKPFVKLKFHDFTQTTVEQSGAGRDLESYRQLLGQAFARGNRPVRLIGVGVRLLDLQGAHEQLRLF

SEQ ID NO: 26 - protein, 366 aa, Artificial Sequence
  EDS054 expressed protein sequence with His6 tag
MHHHHHHGSRKIIHCDCDCFYASIEMRDDPSLRGRPLAVGGRPETRGVVATCNYEARKYGVHSAMSSARAVRLCPDLLIIPPRMEMYRVASAQIMDIYRDYTELVEPLSLDEAYLDVTGSDRLQGSATRIASEIRQRVAQAVGITVSAGVAPSKFVAKIASDWNKPDGLFVVRPQDVDTFVAALPVAKLHGVGKVTGARLKALGVETCADLREWEHDRLRDEFGAFGERLHDLCRGIDLREVSPTRERKSVSVEQTFVTDLHTLEACQALLREMLDQLDARVRRADAQNHIQKLFVKLRFSDFNRTTAEGVGAALDEEQFRILLATAFRRNPRAVRLMGLGVRLGAPGGQLALFGDQPTVSEPDTV

SEQ ID NO: 27 - protein, 510 aa, Artificial Sequence
  EDS066 expressed protein sequence with His6 tag
MHHHHHHGSSFIPLKRRRAGPVSEEPLDSLQSLFPDVCLFLVERRMGSARRKFLTGLAQKKGFCVTPQFSDQVTHVVSEQNSCSEVLLWIERQSGQKVQPGGAEMTPHILDITWFTESMSLGKPVKVEPRHCLGVSDSSVSRDKATQEIPAYGCQRRTPLHHHNKEITDALEILALSASFQGSEARFLGFTRASSVLKSLPFRLQSVEEVKDLPWCGGHSQTVIQEILEDGVCREVETVKNSEHFQSMKALTSIFGVGIRTADKWYRDGVRSLSDLNNLGGKLTAEQKAGLLHYTDLQQSVTREEAGTVEQLIKGALQSFVPDVRVTMTGGFRRGKQEGHDVDFLITHPDEEALNGLLRKAVAWLDGKGSVLYYHVRARSQNFSGSNTMDGHETCYSIIALPNVCPEKPSPDAEKIEPDLDKNSLRNWKAVRVDLVVCPYSEYFYALLGWTGSKHFERELRRFSLHVKKMSLNSHGLFDIQKKCHHPATSEEEIFAHLGLPYVPPSERNA

SEQ ID NO: 28 - protein, 438 aa, Artificial Sequence
  EDS082 expressed protein sequence with His6 tag
MHHHHHHGSEQQKLLDTKEYIIKLDELKPETRKESPASKQHIENLQKQETKEKLIAESSTGNPNERTIFLLNQMAEERLLQGEHFKAKAYKNAINALNNTGDFISDANEALRLKGIGVSVAQKIEEIVKTNTLSSLNEIKSDKEHQVSKLFMGIHGVGPVSAKKWYNDGLRTLEDVSQKPDLTSNQTLGLKYYDEWLERIPRDECTLHNEFMSDLVSQIDPLVQFTIGGSYRRGSPTCGDVDFIITKPNADNEEMKEILEKILVKIEQVGYLKCSLQKKHSTKFLSGCALPPNYASRLPEYSEGKWGKCRRIDFLMVPWKERGAAFIYFTGNDYFNRLIRLKAVKNGLVLNESGLFKRIKYVQGKNVEDKTMLIESFSEKKIFKLLGFKYVPPEQRNFGANNPPSKLGKHLDQFRIDHKYFDKVVKEEIIDDDVIEVD

SEQ ID NO: 29 - protein, 391 aa, Artificial Sequence
  EDS048 expressed protein sequence with His6 tag
MHHHHHHGSRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 30 - protein, 518 aa, Artificial Sequence
  EDS015 expressed protein sequence with His6 tag
MHHHHHHGSDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 31 - DNA, 5515 bp, Artificial Sequence
  PP1077 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcagcaagtttacctggaaagaactgattcagctgggtagcccgagcaaagcatatgaaagcagcctggcatgtattgcccatattgatatgaatgcatttttcgcacaggttgagcagatgcgttgtggtctgagcaaagaagatccggttgtttgcgttcagtggaatagcattattgcagttagctatgcagcccgtaaatatggtattagccgtatggataccattcaagaggcactgaaaaaatgcagcaatctgattccgattcataccgcagttttcaaaaaaggcgaagatttttggcagtatcatgatggttgtggtagctgggttcaagatccggcaaaacaaatttcagtcgaagatcataaagttagcctggaaccgtatcgtcgtgaaagccgtaaagccctgaaaatctttaaaagcgcatgtgatctggttgaacgtgcaagcattgatgaagtttttctggatctgggtcgcatttgttttaacatgctgatgttcgataacgagtatgaactgaccggtgatctgaaactgaaagatgcactgagcaatattcgcgaagcatttattggtggcaactatgatattaacagccatctgccgctgattccggaaaaaatcaaaagcctgaaattcgaaggcgacgtgtttaatccggaaggtcgtgatctgattacagattgggatgatgttattctggcactgggtagtcaggtttgtaaaggtattcgtgatagcatcaaagatatcctgggttataccacctcatgtggtctgtcaagcaccaaaaatgtttgtaaactggccagcaactacaaaaaaccggatgcacagaccattgtgaaaaatgattgtctgctggatttcctggattgcggcaaatttgaaattaccagcttttggaccttaggtggtgttctgggtaaagaattaattgatgtgctggatctgccgcatgaaaacagcattaaacatattcgtgaaacctggcctgataatgcaggtcagctgaaagaatttctggatgccaaagttaaacagagcgattatgatcgtagcaccagcaatattgatccgctgaaaaccgcagatctggccgaaaaactgtttaaactgagccgtggtcgttatggcctgccgctgtcaagccgtccggttgtgaaaagcatgatgagcaataaaaacctgcgtggcaaaagctgcaatagcattgttgattgtattagctggctggaagttttttgtgcagaactgaccagccgtattcaggatctggaacaagaatataacaagatcgttattccgcgtaccgttagcattagcctgaaaaccaaaagctatgaggtgtatcgtaaaagcggtccggtggcatataaaggtatcaattttcagagccacgaactgctgaaagtgggtatcaaatttgtgaccgatctggatatcaaaggcaagaacaaaagttattacccgctgaccaaactgagcatgaccattaccaatttcgatatcatcgatctgcagaaaaccgtggttgatatgtttggtaatcaggtgcatacgtttaaaagcagcgcaggtaaagaagatgaagaaaaaaccaccagtagcaaagccgatgaaaaaaccccgaaactggaatgttgtaaatatcaggttaccttcaccgatcagaaagcactgcaagaacatgcagattatcatctggccctgaaactgtctgaaggtctgaatggtgcagaagaaagcagcaaaaatctgagctttggtgaaaaacgtctgctgtttagccgtaaacgtccgaatagccagcataccgcaacaccgcagaaaaaacaggttaccagcagtaaaaacatcctgagcttttttacccgcaaaaaatgatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 32 - DNA, 5113 bp, Artificial Sequence
  PP1084 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagctttcatgcaaccgcactgcctcgtatgcgtaaacgtccgcgtccggaagaagttgcctgtccgggtcgtgaagatgttaaatttcgtgatgttcgtctgtacctggtggaaatgaaaatgggtcgtagccgtcgtagctttctgacccagctggcacgtagcaaaggttttatggttgaagaggttctgagcaatcgtgttacccatgttgttagcgaaagcagccaggcaccggttctgtgggcatggctgaaagaacgtgcaccgcaggatctgccgaatatgcatgttgtgaatattacctggtttaccgatagcatgcgtgaaagccgtccggttgcagttgaaacccgtcatctgattcaggataccctgcctgcaattccggaaggtggtgcaccggcagccgaagttagccagtatgcatgtcagcgtcgtaccaccaccgataactataatgttgtttttaccgatgcctttgaagttctggccgaatgctatgaatttaatcagatggatggtcgttgtctggcatttcgtcgtgcagcaagcgttctgaaaagcctgcctcgtggtctgagcagcctggaagaaacccatagcctgccgtgtttaggtggtcatgcaaaagcaattattggcgaaattctgcagcatggtcgtgcatttgatgttgaaaaagttctgagtgatgaacgctatcagaccctgaaactgtttaccagcgtttatggtgttggtccgaaaaccgcagaaaaatggtatcgtagcggtctgcgtagcctggatcatattctggcggatcagagcatccagctgaatcatatgcagcagaatggttttctgcattatggtgatattagccgtgcagttagcaaagccgaagcacgtgcactgaccaaagcaattggtgaaaccgttcaggcaattacaccggatgcactgctggcactgaccggtggttttcgtcgcggtaaagaatttggtcatgatgtggatattatctttaccacgctggaattaggcatggaagaaaatctgctgctggcagtgattaaaagtctggaaaaacagggtattctgctgtattgtgattatcaggcaagcacctttgatctgaccaaactgccgacacatagctttgaagcaatggatcattttgccaagtgctttctgattctgcgtctggaagcaagccaggttgaagaaggcctgaatagtccggttgaagatattcgtggttggcgtgcagttcgtgttgatctggttagccctccggttgatcgttatgcatttgcactgttaggttggaccggtagccgtcagtttgaacgtgatctgcgtcgttttgcacgtaaagaacgtcgtatgctgctggataatcatggcctgtatgataaaaccaaagaagaatttctggcagccggtacggaaaaagatatttttgatcatctgggccttgagtatatggaaccgtggcagcgtaatgcataatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 33 - DNA, 5323 bp, Artificial Sequence
  PP1089 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcggtattctgagcggcaaaaaattcctgattctgccgaatagccataccggtagcgttaatattctggcaggtattgttaaagaacaaggtggttttctggttagcagcgcagatcgtctgagcaatgatgttgttgttctggtgaatgatagcttcgtggacaaaaccaacaaaattgttaatcgcggtctgtttctgaaagaatttgaactggatgcaagcgttgtttggacctatgttctggaaaatgaactggtttgtctgcgtgttagcctggttccgagctgggttgaaaatggcacctttcattttagcgatagcgaacgtattattctgctggatagcgaaagccaagaacgcgataccaaaaatgttcagtttcatagcgcaggtaatgaagaggcaggtagtgatgatgaaaccgatgttgaaggtaataaagaaagcaccggtgatattaccgatgttagcgataccgcaacaccgcagctgcagagcagtccgctgagcaaatatatcaaacaagaagaggatatcgacaaccaggttctgattaaagcactgggtcgtctggtgaaaaaatacgaagttaaaggtgatcagtatcgcagccgtagctatcgtctggcaaaacaggcagttgaaaaatatccgcataaaatcaccagcggtagccaggcacagcgtcagctgagcaatattggtagcagcattgccaaaaaaatccagctgctgctggacaccggtacactgcctggtctggaagatccggcaaccgatgaatatgaaagcagcctgggttatttcagcgaatgttatggtattggtgttccgatggccaaaaaatggattaccctgaatatcagcaccttttatcgtgcagcacgtctgcatccgaaactgtttattagcgattggccgattctgtatggctggacctattatgaagattggagcaaacgtattccgcgtgatgaagttaccgcacattttgagctggttaaagaagaagttcgtcgcgttggtaatggttgtagcgttgaaatgcagggtagctatgttcgtggtgcacgtgataccggtgatgttgatctgatgttctacaaagaaaattgcgacgatctggaagaggttaccattggtatggaaaatgttgcagcaagcctgtatcagaaaggctatatcaaatgttttctgctgctgaccgataaactggaacgcatgtttcgtccggatattctgagtcgtctgcagaaatgtggtattgccgaaatcagcaatgaacatacctttcgtaatagcgaccgtggcaaaaaactgtttttcggtgttgaactgccaggcgattatccgatttatccgtttgatgataaagacatcctgcagctgaaaccgcaggataaattcatgagcaaaagcaaagatgccggtcatttttgtcgtcgtctggatttcttctgttgcaaatggtcagaactgggtgcagcccgtattcattataccggtaataccgattataaccgttggctgcgtgttcgtgcaatggatatgggttataaactgacccagcatggcatcttcaaagatgatgtactgctggaaagctttgatgagcgcaaaatctttgaatatctgcatgtgccgtatctgaatccggttgatcgtaataaaaccgattgggtgaatatcccgattccgaaataatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 34 - DNA, 5209 bp, Artificial Sequence
  PP1090 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcaatcgtagcggtcaggttctgagcaaaatgagtaaaacctacctgtttgatggcctggaatttctgtttattccgaacattaatagcagcaaggtgacctttacacgcaaaaatctggcacgtaatggtggtgcaagcgttgccaaaaaattcgatcaggataccaccacacatgttctggttgataccaaagtttatctgaccaaagacaaaattagcgcaggtctgaaaaatgccaaagtgccgaaaacctttcagcctggtaaaattctgaatcagacctggctggttgattctattgaacagcagaaactgctggacaccaaagagtatattatcaaactggatgagctgaaaccggaaacgcgtaaagaaagtccggcaagcaaacagcatattgaaaatctgcagaaacaagaaaccaaagagaaactgattgcagaaagcagcaccggtaatccgaatgaacgtaccatttttctgctgaaccagatggcagaagaacgtctgctgcagggtgaacattttaaagcaaaagcctataagaacgccattaacgccctgaataataccggtgattttatctcagatgcaaatgaagcactgcgcctgaaaggtattggtgttagcgtggcacagaaaattgaagaaattgtgaaaaccaatacgctgagcagcctgaatgaaatcaaaagcgataaagaacaccaggtgagcaaactgtttatgggtattcatggtgttggtccggttagcgcaaaaaagtggtataatgatggtctgcgtaccctggaagatgttagccagaaaccggatctgaccagcaatcagaccctgggcctgaaatattacgatgaatggctggaacgtattccgcgtgatgaatgtaccctgcataatgaatttatgagcgatctggtgagccagattgatccgctggttcagtttaccattggtggtagctatcgtcgtggtagcccgacctgtggtgatgtggattttatcattaccaaaccgaatgccgataacgaagagatgaaagagattctggaaaagatcctggtgaaaatcgaacaggttggttatctgaaatgtagcctgcagaaaaaacacagcaccaaatttctgagcggttgtgcactgcctccgaattatgcaagccgtctgccggaatacagcgaaggtaaatggggtaaatgtcgtcgtattgattttctgatggttccgtggaaagaacgtggtgcagcatttatctattttaccggcaacgattatttcaaccgtctgattcgtctgaaagccgttaaaaatggtctggtgctgaatgaatcaggtctgtttaaacgcatcaaatacgtgcagggtaaaaacgtggaagataaaaccatgctgatcgaaagctttagcgagaaaaaaatctttaagctgctgggcttcaaatatgttccgcctgaacagcgtaattttggtgcaaataatccgcctagcaaactgggtaaacatctggatcagtttcgcatcgatcacaaatatttcgacaaagtggtgaaagaagagatcattgacgacgatgttatcgaggtggattaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 35 - DNA, 4666 bp, Artificial Sequence
  PP1113 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagccgcaaaatcatccatattgattgcgattgcttttacgcagcactggaaatgcgtgatgatccgagcctgcgtggtaaagcactggcagttggtggtagtccggataaacgtggtgttgttgcaacctgtagctatgaagcacgtgcatatggtgttcgtagcgcaatggcaatgcgtaccgcactgaaactgtgtccggatctgctggttgttcgtccgcgttttgatgtttatcgtgcagttagcaaacaaatccatgccatctttcgtgattataccgatctgattgaaccgctgagcctggatgaagcatatctggatgttagcgcaagtccgcattttgcaggtagcgcaacccgtattgcacaggatattcgtcgtcgtgttgcagaagaactgcgtattaccgttagtgccggtgttgcaccgaacaaatttctggcaaaaattgcaagcgattggcgtaaaccggatggtctgtttgttattacaccggaacaggttgatggttttgttgccgaactgccggttgcaaaactgcatggtgttggtaaagttaccgcagaacgtctggcacgtatgggtattcgtacctgtgccgatctgcgtcagggtagcaaactgagtctggttcgtgaatttggtagctttggtgaacgtctgtggggtttagcacatggtattgatgaacgtccggttgaagttgatagccgtcgtcagagcgttagcgttgaatgtacctttgatcgtgatctgccggatctggcagcatgtctggaagaattaccgacactgctggaagaactggatggtcgtctgcagcgtctggatggtagctatcgtcctgataaaccgtttgtgaaactgaaattccacgattttacccagaccaccgttgaacagagcggtgcaggtcgcgatctggaaagttatcgtcagctgctgggtcaagcatttgcacgtggtaatcgtccggttcgtctgattggtgtgggtgttcgtctgctggatctgcagggtgcacatgaacagctgcgtctgttttaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 36 - DNA, 4693 bp, Artificial Sequence
  PP1114 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagccgcaaaatcattcattgtgattgcgattgcttttacgccagcattgaaatgcgtgatgatccgagcctgcgtggtcgtccgctggcagttggtggccgtccggaaacacgtggtgttgttgcaacctgtaattatgaagcacgtaaatatggtgttcatagcgcaatgagcagcgcacgtgcagttcgtctgtgtccggatctgctgattattccgcctcgtatggaaatgtatcgtgttgcaagcgcacagatcatggatatttatcgtgattataccgaactggttgaaccgctgagcctggatgaagcatatctggatgttaccggtagcgatcgtctgcagggtagcgcaacccgtattgcaagcgaaattcgtcagcgtgttgcacaggccgttggtattaccgttagtgccggtgttgcaccgagcaaatttgttgccaaaattgccagcgattggaataaaccggatggtctgtttgttgttcgtccgcaggatgttgatacctttgttgcagcactgccggttgcaaaactgcatggtgttggtaaagttaccggtgcacgtctgaaagcactgggtgttgaaacctgtgccgatctgcgtgaatgggaacatgatcgtttacgtgatgaatttggtgcatttggtgaacgtctgcacgatctgtgtcgtggtattgatctgcgcgaagttagcccgacacgtgaacgtaaaagcgttagcgttgaacagacctttgttaccgatctgcataccctggaagcatgtcaggcactgctgcgtgaaatgctggatcagctggatgcacgtgttcgtcgtgcagatgcacagaaccatattcagaaactgtttgtgaaactgcgcttcagcgattttaatcgtaccacagccgaaggtgttggtgccgcactggatgaggaacagtttcgtattctgctggcaaccgcatttcgtcgtaatccgcgtgccgtgcgtctgatgggtctgggtgttcgtctgggtgcacctggtggtcagctggcactgtttggtgatcagccgaccgttagcgaaccggataccgtttaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 37 - DNA, 5125 bp, Artificial Sequence
  PP1126 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcagctttattccgctgaaacgtcgtcgtgcaggtccggttagcgaagaaccgctggatagcctgcagagcctgtttccggatgtttgtctgtttctggttgaacgtcgtatgggtagcgcacgtcgtaaatttctgaccggtctggcacagaaaaaaggtttttgtgttacaccgcagtttagcgatcaggttacccatgttgttagcgaacagaatagctgtagcgaagttctgctgtggattgaacgtcagagtggtcagaaagttcagcctggtggtgcagaaatgacaccgcatattctggatattacctggtttaccgaaagcatgagcctgggtaaaccggttaaagttgaaccgcgtcattgtctgggtgttagcgatagcagcgttagccgtgataaagcaacccaagaaattccggcatatggttgtcagcgtcgtacaccgctgcatcatcataataaagaaattaccgatgcgctggaaattctggcactgagcgcaagctttcagggtagcgaagcacgttttctgggttttacccgtgcaagcagcgttctgaaaagcctgccgtttcgtctgcagagcgttgaagaggttaaagatctgccgtggtgtggtggtcatagccagaccgttattcaagaaatcctggaagatggtgtttgccgtgaagttgaaaccgtgaaaaatagcgaacatttccagagcatgaaagcactgaccagcatttttggtgttggtattcgtaccgcagataaatggtatcgtgatggtgttcgtagcctgagcgatctgaataatcttggtggtaaactgaccgcagaacagaaagcaggtctgctgcattacaccgatctgcagcagagcgtgacccgtgaagaagcaggcaccgttgaacagctgattaaaggtgcactgcagagctttgtgccggatgtgcgtgttaccatgaccggtggttttcgtcgtggtaaacaagagggtcatgatgtggattttctgattacccatcctgatgaagaagccctgaacggcctgctgcgtaaagcagttgcatggctggatggtaaaggtagcgttctgtattatcatgttcgtgcacgtagtcagaattttagcggtagcaataccatggatggtcatgaaacctgttatagcattattgcactgccgaatgtttgtccggaaaaaccgagtccggatgcagaaaaaattgaaccggatctggataaaaacagcctgcgtaattggaaagcagttcgtgttgatctggttgtttgcccgtatagcgaatacttttatgcactgttaggttggaccggcagcaaacattttgaacgtgaactgcgtcgttttagcctgcatgtgaaaaaaatgagcctgaatagccatggcctgtttgacattcagaaaaagtgtcatcatccggcaaccagcgaagaagaaatttttgcacatctgggtctgccgtatgttccgcctagcgaacgtaatgcataatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 38 - DNA, 4909 bp, Artificial Sequence
  PP1142 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcgaacagcagaaactgctggacaccaaagagtatattatcaaactggatgagctgaaaccggaaacgcgtaaagaaagtccggcaagcaaacagcatattgaaaatctgcagaaacaagaaaccaaagagaaactgattgcagaaagcagcaccggtaatccgaatgaacgtaccatttttctgctgaaccagatggcagaagaacgtctgctgcagggtgaacattttaaagcaaaagcctataagaacgccattaacgccctgaataataccggtgattttatctcagatgcaaatgaagcactgcgcctgaaaggtattggtgttagcgtggcacagaaaattgaagaaattgtgaaaaccaatacgctgagcagcctgaatgaaatcaaaagcgataaagaacaccaggtgagcaaactgtttatgggtattcatggtgttggtccggttagcgcaaaaaagtggtataatgatggtctgcgtaccctggaagatgttagccagaaaccggatctgaccagcaatcagaccctgggcctgaaatattacgatgaatggctggaacgtattccgcgtgatgaatgtaccctgcataatgaatttatgagcgatctggtgagccagattgatccgctggttcagtttaccattggtggtagctatcgtcgtggtagcccgacctgtggtgatgtggattttatcattaccaaaccgaatgccgataacgaagagatgaaagagattctggaaaagatcctggtgaaaatcgaacaggttggttatctgaaatgtagcctgcagaaaaaacacagcaccaaatttctgagcggttgtgcactgcctccgaattatgcaagccgtctgccggaatacagcgaaggtaaatggggtaaatgtcgtcgtattgattttctgatggttccgtggaaagaacgtggtgcagcatttatctattttaccggcaacgattatttcaaccgtctgattcgtctgaaagccgttaaaaatggtctggtgctgaatgaatcaggtctgtttaaacgcatcaaatacgtgcagggtaaaaacgtggaagataaaaccatgctgatcgaaagctttagcgagaaaaaaatctttaagctgctgggcttcaaatatgttccgcctgaacagcgtaattttggtgcaaataatccgcctagcaaactgggtaaacatctggatcagtttcgcatcgatcacaaatatttcgacaaagtggtgaaagaagagatcattgacgacgatgttatcgaggtggattaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 39 - DNA, 4768 bp, Artificial Sequence
  PP1108 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagccgtaccgattatagcgcaaccccgaatccgggttttcagaaaacaccgcctctggcagtgaaaaaaatcagccagtatgcatgtcagcgtaaaaccacactgaataactataaccacatcttcaccgatgcctttgaaattctggcagaaaacagcgaattcaaagaaaacgaagttagctacgtgacctttatgcgtgcagcaagcgttctgaaaagcctgccgtttaccattattagcatgaaagataccgaaggtattccgtgtctgggtgataaagtgaaatgcatcattgaagagatcatcgaagatggtgaaagcagcgaagttaaagcagttctgaatgatgaacgttaccagagcttcaaactgtttaccagcgtttttggtgttggcctgaaaaccagcgaaaaatggtttcgtatgggttttcgtagcctgagcaaaatcatgagcgataaaaccctgaaattcaccaaaatgcagaaagccggtttcctgtattatgaagatctggtgagctgtgttacccgtgccgaagccgaagcagttggtgttctggttaaagaagcagtttgggcatttctgccggatgcatttgttaccatgaccggtggttttcgtcgtggcaaaaaaatcggtcatgatgtggattttctgattaccagtccgggtagcgcagaagatgaagaacagctgctgccgaaagttattaatctgtgggaaaaaaaaggcctgctgctgtattacgatctggttgaaagcaccttcgagaaattcaaactgccgagccgtcaggttgataccctggatcactttcagaaatgttttcttatcctgaagctgcatcatcagcgtgttgatagcagcaaaagcaatcagcaagaaggtaaaacctggaaagcaattcgtgttgatctggttatgtgcccgtatgaaaatcgtgcatttgcactgttaggttggaccggtagtcgtcagtttgaacgtgatattcgtcgttatgcaacccatgaacgtaaaatgatgctggataatcatgccctgtacgataaaacgaaacgcgtgttcctgaaagccgaaagcgaagaagaaatttttgcacatctgggccttgattacattgaaccgtgggaacgtaatgcctaatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 40 - DNA, 5149 bp, Artificial Sequence
  PP1075 expression vector full sequence
aacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagaagatctcgatccgcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgcatcatcatcaccatcacggcagcgatccgctgcaggcagttcatctgggtccgcgtaaaaaacgtccgcgtcagctgggtacaccggttgcaagcaccccgtatgatattcgttttcgtgatctggttctgttcatcctggaaaaaaagatgggtacaacccgtcgtgcatttctgatggaactggcacgtcgtaaaggttttcgtgttgaaaatgaactgagcgatagcgttacccatattgttgcagaaaataacagcggtagtgatgttctggaatggctgcaactgcagaacattaaagcaagcagcgaactggaactgctggatattagctggctgattgaatgtatgggtgcaggtaaaccggttgaaatgatgggtcgtcatcagctggttgttaatcgtaatagcagcccgagtccggttccgggtagccagaatgttccggcaccggcagtgaaaaaaatcagtcagtatgcatgtcagcgtcgtaccacactgaataactataatcagctgtttaccgatgcactggatattctggcagaaaatgatgagctgcgcgaaaatgaaggtagctgtctggcatttatgcgtgccagcagcgttctgaaaagcctgccgtttccgattaccagcatgaaagataccgaaggtattccgtgtctgggtgataaagtgaaaagcattattgaaggcatcatcgaagatggcgaaagcagtgaagcaaaagcagttctgaatgatgaacgctacaaaagcttcaaactgtttaccagcgtttttggtgttggtctgaaaaccgcagaaaaatggtttcgtatgggttttcgtaccctgagcaaaattcagagcgataaaagtctgcgttttacccagatgcagaaagcaggttttctgtattatgaagatctggtgagctgcgttaatcgtccggaagccgaagcagttagcatgctggttaaagaagcagttgttacctttctgccggatgcgctggttaccatgaccggtggttttcgtcgcggaaaaatgacaggtcatgatgtggattttctgattacctcaccggaagcaaccgaagatgaagaacagcaactgctgcataaagttaccgatttttggaaacagcagggtctgctgctgtattgtgatatcctggaatcaaccttcgagaaattcaaacagccgagccgtaaagttgatgccctggatcattttcagaagtgttttctgatcctgaaactggatcatggtcgtgttcatagcgaaaaaagcggtcagcaagaaggtaaaggttggaaagcaattcgtgtggatctggttatgtgtccgtatgatcgtcgtgcctttgcactgttaggttggaccggtagccgtcagtttgaacgtgatctgcgtcgttatgcaacccatgaacgtaaaatgatgctggataatcatgcactgtatgatcgcaccaaacgtgtttttctggaagcagaaagcgaagaagaaatctttgcacatctgggccttgattacattgaaccgtgggaacgtaatgcataatgcacgtgaggatccaactcgagaacttagatggtattagtgacctgtaacagagcattagcgcaaggtgatttttgtcttcttgcgctaattttttgtcatcaaacctgtcgctagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 41 - DNA, 18 bp, Artificial Sequence
  PG1350 oligonucleotide
gcgtcacgctaccaacca

SEQ ID NO: 42 - DNA, 20 bp, Artificial Sequence
  PG5858 oligonucleotide
gtcctcaatcgcactggaaa

SEQ ID NO: 43 - DNA, 20 bp, Artificial Sequence
  PG5859 oligonucleotide
gtcctcaatcgcactggaag

SEQ ID NO: 44 - DNA, 20 bp, Artificial Sequence
  PG5860 oligonucleotide
gtcctcaatcgcactggaac

SEQ ID NO: 45 - DNA, 20 bp, Artificial Sequence
  PG5861 oligonucleotide
gtcctcaatcgcactggaat

SEQ ID NO: 46 - DNA, 21 bp, Artificial Sequence
  PG5864 oligonucleotide
gtcctcaatcgcactggaatt

SEQ ID NO: 47 - DNA, 22 bp, Artificial Sequence
  PG5865 oligonucleotide
gtcctcaatcgcactggaattg

SEQ ID NO: 48 - DNA, 23 bp, Artificial Sequence
  PG5866 oligonucleotide
gtcctcaatcgcactggaattga

SEQ ID NO: 49 - DNA, 21 bp, Artificial Sequence
  PG5868 oligonucleotide
gtcctcaatcgcactggaagt

SEQ ID NO: 50 - DNA, 21 bp, Artificial Sequence
  PG5869 oligonucleotide
gtcctcaatcgcactggaagc

SEQ ID NO: 51 - DNA, 30 bp, Artificial Sequence
  PG5870 oligonucleotide
gtcctcaatcgcactggaaacatcaaggtc

SEQ ID NO: 52 - DNA, 40 bp, Artificial Sequence
  PG5871 oligonucleotide
gtcctcaatcgcactggaaacatcaaggtcatacggaacg

SEQ ID NO: 53 - DNA, 21 bp, Artificial Sequence
  PG5872 oligonucleotide
gtcctcaatcgcactggaatg

SEQ ID NO: 54 - DNA, 24 bp, Artificial Sequence
  PG5867 oligonucleotide
gtcctcaatcgcactggaattgac